(19) United States
(12) Patent Application Publication
(10) Pub. No.: US 2024/0350630 A1
(43) Pub. Date: Oct. 24, 2024

(54) COMPOSITIONS AND METHODS OF TREATING DISEASE WITH CHIMERIC ANTIGEN RECEPTORS TO B CELL MATURATION ANTIGEN (BCMA)

(71) Applicant: AstraZeneca AB, Sodertalje (SE)

(72) Inventors: Jessica Gayle Tong, Gaithersburg, MD (US); Deepali Malhotra, Wilmington, DE (US); Michael Overstreet, Wilmington, DE (US); Gordon Moody, Wilmington, DE (US); Mark Cobbold, Wilmington, DE (US); Laura Vian, Wilmington, DE (US); Gianluca Carlesso, Gaithersburg, MD (US); Attilio Bondanza, Cambridge (GB); Ryan Nicholas Gilbreth, Gaithersburg, MD (US)

(21) Appl. No.: 18/583,233

(22) Filed: Feb. 21, 2024

Related U.S. Application Data
(60) Provisional application No. 63/486,391, filed on Feb. 22, 2023.

(51) Int. Cl. A61K 39/00; A61P 35/00; A61P 37/06; C07K 14/705; C07K 14/715; C07K 14/725; C07K 14/73; C07K 16/28

(52) U.S. Cl. CPC A61K 39/46433; A61K 39/4631; A61P 35/00; A61P 37/06; C07K 14/70507; C07K 14/7051; C07K 14/70514; C07K 14/70517; C07K 14/70521; C07K 14/70578; C07K 14/7155; C07K 16/2878; C07K 2319/03

(57) ABSTRACT

This disclosure relates to compositions and methods for treating disease using chimeric antigen receptor cells and/or antigen binding domains targeting BCMA.

### CROSS REFERENCE TO RELATED APPLICATIONS

This application claims priority to U.S. Provisional Application No. 63/486,391, filed Feb. 22, 2023, the disclosure of which is incorporated by reference in its entirety.

### REFERENCE TO SEQUENCE LISTING

This application is being filed electronically and includes an electronically submitted sequence listing. The sequence listing is entitled “23-0105-WO_SequenceListing.xml” and was created on Feb. 19, 2024, and has a size of 90,152 bytes. The sequence listing contained in this.xml file is part of the specification and is herein incorporated by reference in its entirety.

### BACKGROUND OF DISCLOSURE

### Field of Invention

This disclosure relates to treatment of cancer and other diseases using BCMA-targeting chimeric antigen receptor cells.

### Technical Background

B-cell maturation antigen (BCMA) is a tumor necrosis family receptor (TNFR) member expressed on cells of the B-cell lineage (Laabi et al., Nucleic Acids Research, 22 (7): 1147-1154 (1994)). BCMA expression is highest on terminally differentiated B cells, and BCMA is involved in mediating the survival of plasma cells for maintaining long-term humoral immunity. The expression of BCMA has been linked to a number of cancers, autoimmune disorders, and infectious diseases. In particular, BCMA RNA has been detected universally in multiple myeloma cells, and BCMA protein has been detected on the surface of plasma cells from multiple myeloma patients by several investigators (see, e.g., Novak et al, Blood, 103 (2): 689-694 (2004); Neri et al., Clinical Cancer Research, 73 (19): 5903-5909 (2007); Bellucci et al., Blood, 105 (10): 3945-3950 (2005); and Moreaux et al., Blood, 703 (8): 3148-3157 (2004)). As such, BCMA has been investigated as a possible therapeutic target for multiple myeloma and other diseases.

Chimeric antigen receptor (CAR)-based cell therapy is a specific form of cell-based immunotherapy that uses engineered immune cells to fight disease. Such cellular therapies have been transformative for patients with hematological malignancies in recent years with the first approval for a CAR-based therapy by the FDA in 2017 (Larson & Maus, Nat Rev Cancer 21, 145-161 (2021); Yu, et al., Nature Reviews Drug Discovery 19, 583-584 (2020)). Moreover, the number of clinical trials investigating adoptive cell therapies has grown rapidly over the past several years.

While there is much potential for cellular therapies to be curative for patients, a number of factors limit the widespread development and administration of these drugs. Most cellular therapies are currently produced in an autologous fashion and are associated with variable cell product quality, cytokine release syndrome and other toxicities, extended manufacturing times, complicated supply chain logistics, high costs, and a limited period in which these therapies may be genetically modified to enhance their efficacy (Larson &Maus, Nat Rev Cancer 21, 145-161 (2021)).

In particular, autologous cell therapies with primary human immune cells (e.g., T cells and NK cells) possess a finite potential for proliferation. This significantly restricts the window for the cells to be isolated, expanded, manufactured, and genetically edited, while still retaining function upon reinfusion into patients. Proloning the life span of these cells through deletion of cell cycle related genes broadens the window for manufacturing, allowing for several manipulations that can mitigate cytokine release syndrome and other toxicities, enable armoring of the cells, and most critically, allow for the generation of large cell banks of allogeneically edited cells for widespread distribution.

Current FDA approved BCMA CAR-T cell therapies for Multiple Myeloma (MM) are autologous products that have been transformative for patients that are refractory to all other available treatments with reported overall response rates of up to 95%. However, the practical challenges for delivering an autologous CAR-T cell product have severely restricted the enrollment of qualifying patients. Moreover, toxicities related to BCMA CAR-T cell therapies and BCMA T cell engager-based therapies also exceed >90% of those treated. Therefore, alternative BCMA CAR cell-based therapies are needed to address these manufacturing constraints while minimizing the associated toxicities.

### SUMMARY

This disclosure describes compositions and methods for using CAR-based cell therapies to treat cancer and other diseases.

As described below, in a first aspect, the disclosure provides an isolated nucleic acid sequence encoding a chimeric antigen receptor (CAR), wherein the CAR comprises:
(a) an antigen binding domain specific for B cell maturation antigen (BCMA);
(b) a transmembrane domain; and
(c) one or more intracellular domains.

In some embodiments of the isolated nucleic acid sequence, the antigen binding domain comprises an antibody or antigen-binding fragment thereof, Fab, Fab′, F(ab′)2, Fd, Fv, single-chain fragment variable (scFv), single chain antibody, VHH, vNAR, nanobody (single-domain antibody), or any combination thereof. In some embodiments, the antigen binding domain is a single chain variable fragment (scFv). In some embodiments, the antigen binding domain is a scFv comprising an amino acid sequence selected from SEQ ID NO: 9, 36, and 90. In an embodiment of the isolated nucleic acid sequence, the antigen binding domain is a scFv comprising the amino acid sequence of SEQ ID NO: 9.

In some embodiments of the isolated nucleic acid sequence, the transmembrane domain comprises a transmembrane domain selected from the transmembrane domain of CD4, CD8α, or CD28. In an embodiment, the transmembrane domain comprises a CD28 transmembrane domain.

In some embodiments of the isolated nucleic acid sequence, the one or more intracellular domains comprises a costimulatory domain or a portion thereof. In some embodiments, the costimulatory domain comprises one or more of CD32, 4-1BB, CD2, CD27, CD28, OX-40, ICOS, IL-2Rβ, GITR, MyD88/CD40a costimulatory domains, and/or variants thereof. In an embodiment of the isolated nucleic acid sequence, the intracellular domain comprises a CD3z costimulatory domain and a CD28 costimulatory domain. In another embodiment of the isolated nucleic acid sequence, the intracellular domain comprises a CD3z costimulatory domain and a 4-1BB costimulatory domain. In yet another embodiment of the isolated nucleic acid sequence, the intracellular domain comprises a CD32 costimulatory domain, a CD28 costimulatory domain, and a 4-1BB costimulatory domain.

In some embodiments of the isolated nucleic acid sequence, the CAR further comprises a hinge/spacer domain, optionally, wherein the hinge/spacer domain is located between the antigen binding domain and the transmembrane domain. In some embodiments, the hinge/spacer domain comprises an IgG1 hinge domain or variants thereof, an IgG2 hinge domain or variants thereof, an IgG3 hinge domain or variants thereof, an IgG4 hinge domain or variants thereof, an IgG4P domain, a CD8 hinge domain or variants thereof or a CD28 hinge domain or variants thereof. In an embodiment, the hinge/spacer domain is an IgG4 hinge/spacer, or variants thereof, optionally an IgG4P hinge/spacer comprising an S241P mutation.

In an embodiment of the isolated nucleic acid sequence, the nucleic acid sequence encodes a CAR with an amino acid sequence as set forth in SEQ ID NO: 96.

In another embodiment of the isolated nucleic acid sequence, the nucleic acid sequence encodes a CAR with an amino acid sequence as set forth in SEQ ID NO: 97.

In yet another embodiments of the isolated nucleic acid sequence, the nucleic acid sequence encodes a CAR with an amino acid sequence as set forth in SEQ ID NO: 98.

In another aspect, this disclosure provides an anti-BCMA chimeric antigen receptor (CAR) comprising an antigen binding domain, wherein the antigen binding domain comprises an antibody, Fab, or an scFv comprising a heavy chain variable region (VH) and a light chain variable region (VL);
wherein the VH comprises a CDR1 comprising an amino acid sequence selected from SEQ ID NO: 2, 29, and 83; a CDR2 comprising an amino acid sequence selected from SEQ ID NO: 3, 30, and 84; and a CDR3 comprising an amino acid sequence selected from SEQ ID NO: 4, 31, and 85; and
wherein the VL comprises a CDR1 comprising an amino acid sequence selected from SEQ ID NO: 6, 33, and 87; a CDR2 comprising an amino acid sequence selected from SEQ ID NO: 7, 34, and 88; and a CDR3 comprising an amino acid sequence selected from SEQ ID NO: 8, 35, and 89.

In some embodiments of the anti-BCMA CAR, the VH comprises an amino acid sequence selected from SEQ ID NO: 1, 28, and 82. In some embodiments of the anti-BCMA CAR, the VL comprises an amino acid sequence selected from SEQ ID NO: 5, 32, and 86.

In an aspect, this disclosure provides an anti-BCMA chimeric antigen receptor (CAR) comprising an antigen binding domain, wherein the antigen binding domain comprises an antibody, Fab, or an scFv comprising a heavy chain variable region (VH) and a light chain variable region (VL);
wherein the VH comprises a CDR1 comprising the amino acid sequence of SEQ ID NO: 2; a CDR2 comprising the amino acid sequence of SEQ ID NO: 3; and a CDR3 comprising the amino acid sequence of SEQ ID NO: 4; and
wherein the VL comprises a CDR1 comprising the amino acid sequence of SEQ ID NO: 6; a CDR2 comprising the amino acid sequence of SEQ ID NO: 7; and a CDR3 comprising the amino acid sequence of SEQ ID NO: 8.

In some embodiments of the anti-BCMA CARs disclosed herein, the VH comprises the amino acid sequence of SEQ ID NO: 1, and the VL comprises the amino acid sequence of SEQ ID NO: 5.

In some embodiments of the anti-BCMA CARs disclosed herein, the CAR comprises a transmembrane domain, and one or more intracellular domains. In some embodiments, the transmembrane domain comprises a transmembrane domain selected from the transmembrane domain of CD4, CD8α, or CD28. In some embodiments, the transmembrane domain comprises a CD28 transmembrane domain.

In some embodiments of the anti-BCMA CARs disclosed herein, the one or more intracellular domains comprises a costimulatory domain or a portion thereof. In some embodiments, the costimulatory domain comprises one or more of CD3z, 4-1BB, CD2, CD27, CD28, OX-40, ICOS, IL-2Rβ, GITR, MyD88/CD40a costimulatory domains, and/or variants thereof.

In an embodiment of the anti-BCMA CAR disclosed herein, intracellular domain comprises a CD3z costimulatory domain and a CD28 costimulatory domain.

In another embodiment of the anti-BCMA CAR disclosed herein, the intracellular domain comprises a CD3z costimulatory domain and a 4-1BB costimulatory domain.

In yet another embodiment of the anti-BCMA CARs disclosed herein, the intracellular domain comprises a CD3z costimulatory domain, a CD28 costimulatory domain, and a 4-1BB costimulatory domain.

In some embodiments of the anti-BCMA CARs disclosed herein, the CAR further comprises a hinge/spacer domain, optionally, wherein the hinge/spacer domain is located between the antigen binding domain and the transmembrane domain. In some embodiments, the hinge/spacer domain comprises an IgG1 hinge domain or variants thereof, an IgG2 hinge domain or variants thereof, an IgG3 hinge domain or variants thereof, an IgG4 hinge domain or variants thereof, an IgG4P domain, a CD8a hinge domain or variants thereof, or a CD28 hinge domain or variants thereof. In an embodiment, the hinge/spacer domain is an IgG4 hinge/spacer, or variants thereof, optionally an IgG4P hinge/spacer comprising an S241P mutation.

In an embodiment of the anti-BCMA CAR disclosed herein, the CAR has an amino acid sequence as set forth in SEQ ID NO: 96.

In another embodiment of the anti-BCMA CAR disclosed herein, the CAR has an amino acid sequence as set forth in SEQ ID NO: 97.

In yet another embodiment of the anti-BCMA CAR disclosed herein, the CAR has an amino acid sequence as set forth in SEQ ID NO: 98.

In another aspect, this disclosure provides for a vector comprising the isolated nucleic acid sequence as disclosed herein or encoding the chimeric antigen receptor of as disclosed herein, optionally wherein the vector is a virus, a lentivirus, an adenovirus, a retrovirus, an adeno-associated virus (AAV), a transposon, a DNA vector, a mRNA, a lipid nanoparticle (LNP), or a CRISPR-Cas System.

In yet another aspect, this disclosure provides for a cell comprising the vector as disclosed herein.

In another aspect, this disclosure provides a cell comprising a nucleic acid sequence encoding the chimeric antigen receptor (CAR) as disclosed herein further comprising decreased expression or knock out of one or more endogenous regulatory factors.

In some embodiments of the cell as disclosed herein, the one or more endogenous regulatory factors are selected from cyclin-dependent kinase inhibitor 2A (CDKN2A), cyclin-dependent kinase inhibitor 2B (CDKN2B), and S-methyl-5′-thioadenosine phosphorylase (MTAP).

In some embodiments of the cell as disclosed herein, the cell has decreased expression or knock out of CDKN2A, CDKN2B, and MTAP.

In some embodiments of the cell as disclosed herein, the cell does not express phosphatase and tensin homolog (PTEN).

In some embodiments of the cell as disclosed herein, the cell further comprises a transgene encoding either B-cell lymphoma-extra large (Bcl-xL) or B-cell lymphoma 2 (Bcl-2).

In some embodiments of the cell as disclosed herein, cell does not express of one or more endogenous immune related genes. In some embodiments, the endogenous immune related gene is beta-2 microglobulin (B2M), and/or T-cell receptor a constant (TRAC).

In some embodiments of the cell as disclosed herein, the cell does not express cluster of differentiation 38 (CD38).

In an aspect, this disclosure provides a cell comprising a BCMA specific antigen binding domain, wherein the antigen binding domain comprises an antibody, Fab, or an scFv comprising a heavy chain variable region (VH) and a light chain variable region (VL);
wherein the VH comprises a CDR1 comprising an amino acid sequence selected from SEQ ID NO: 2, 29, and 83; a CDR2 comprising an amino acid sequence selected from SEQ ID NO: 3, 30, and 84; and a CDR3 comprising an amino acid sequence selected from SEQ ID NO: 4, 31, and 85; and
wherein the VL comprises a CDR1 comprising an amino acid sequence selected from SEQ ID NO: 6, 33, and 87; a CDR2 comprising an amino acid sequence selected from SEQ ID NO: 7, 34, and 88; and a CDR3 comprising an amino acid sequence selected from SEQ ID NO: 8, 35, and 89.

In some embodiments of the cell as disclosed herein, the VH comprises an amino acid sequence selected from SEQ ID NO: 1, 28, and 82. In some embodiments of the cell as disclosed herein, the VL comprises an amino acid sequence selected from SEQ ID NO: 5, 32, and 86.

In another aspect, this disclosure provides for a cell comprising a BCMA specific antigen binding domain, wherein the antigen binding domain comprises an antibody, Fab, or an scFv comprising a heavy chain variable region (VH) and a light chain variable region (VL);
wherein the VH comprises a CDR1 comprising the amino acid sequence of SEQ ID NO: 2; a CDR2 comprising the amino acid sequence of SEQ ID NO: 3; and a CDR3 comprising the amino acid sequence of SEQ ID NO: 4; and
wherein the VL comprises a CDR1 comprising the amino acid sequence of SEQ ID NO: 6; a CDR2 comprising the amino acid sequence of SEQ ID NO: 7; and a CDR3 comprising the amino acid sequence of SEQ ID NO: 8.

In some embodiments of the cells as disclosed herein, the VH comprises the amino acid sequence of SEQ ID NO: 1, and the VL comprises the amino acid sequence of SEQ ID NO: 5.

In some embodiments of the cells as disclosed herein, the cells further comprise decreased expression or knock out of one or more endogenous regulatory factors. In some embodiments, the one or more endogenous regulatory factors are selected from cyclin-dependent kinase inhibitor 2A (CDKN2A), cyclin-dependent kinase inhibitor 2B (CDKN2B), and S-methyl-5′-thioadenosine phosphorylase (MTAP). In some embodiments, the cell has decreased expression or knock out of CDKN2A, CDKN2B, and MTAP.

In some embodiments of the cells as disclosed herein, the cell does not express phosphatase and tensin homolog (PTEN).

In some embodiments of the cells as disclosed herein, the cell further comprises a transgene encoding either B-cell lymphoma-extra large (Bcl-xL) or B-cell lymphoma 2 (Bcl-2).

In some embodiments of the cells as disclosed herein, the cell does not express of one or more endogenous immune related genes. In some embodiments, the endogenous immune related gene is beta-2 microglobulin (B2M), and/or T-cell receptor a constant (TRAC).

In some embodiments of the cells as disclosed herein, the cell does not express cluster of differentiation 38 (CD38).

In another aspect, this disclosure provides for a cell comprising a BCMA specific antigen binding domain, wherein the antigen binding domain comprises an antibody, Fab, or an scFv comprising a heavy chain variable region (VH) and a light chain variable region (VL);
wherein the VH comprises a CDR1 comprising the amino acid sequence of SEQ ID NO: 2; a CDR2 comprising the amino acid sequence of SEQ ID NO: 3; and a CDR3 comprising the amino acid sequence of SEQ ID NO: 4; and
wherein the VL comprises a CDR1 comprising the amino acid sequence of SEQ ID NO: 6; a CDR2 comprising the amino acid sequence of SEQ ID NO: 7; and a CDR3 comprising the amino acid sequence of SEQ ID NO: 8; and
wherein the cell comprises decreased expression or knock out of CDKN2A, CDKN2B, MTAP, B2M, TRAC, and CD38.

In an embodiment of the cells as disclosed herein, the VH comprises the amino acid sequence of SEQ ID NO: 1, and the VL comprises the amino acid sequence of SEQ ID NO: 5.

In an embodiment of the cells as disclosed herein, the BCMA specific antigen binding domain comprises an amino acid sequence as set forth in SEQ ID NO: 96.

In some embodiments of the cells as disclosed herein, the cell is selected from a T cell, a Natural Killer (NK) cell, a cytotoxic T lymphocyte (CTL), a tumor infiltrating lymphocyte, a regulatory T cell, a CD8+ T cell, a CD4+ T cell, a gamma-delta T cell, a mucosal associated invariant T (MAIT) T cell, a natural killer T (NKT) cell, and/or a combination thereof.

In an aspect, this disclosure provides a method of treating a disease, comprising: administering to a subject in need thereof an effective amount of a cell comprising an anti-BCMA chimeric antigen receptor (CAR) comprising an antigen binding domain, wherein the antigen binding domain comprises an antibody, Fab, or an scFv comprising a heavy chain variable region (VH) and a light chain variable region (VL),
wherein the VH comprises a CDR1 comprising an amino acid sequence selected from SEQ ID NO: 2, 29, and 83; a CDR2 comprising an amino acid sequence selected from SEQ ID NO: 3, 30, and 84; and a CDR3 comprising an amino acid sequence selected from SEQ ID NO: 4, 31, and 85; and
wherein the VL comprises a CDR1 comprising an amino acid sequence selected from SEQ ID NO: 6, 33, and 87; a CDR2 comprising an amino acid sequence selected from SEQ ID NO: 7, 34, and 88; and a CDR3 comprising an amino acid sequence selected from SEQ ID NO: 8, 35, and 89.

In some embodiments of the methods as disclosed herein, the VH comprises an amino acid sequence selected from SEQ ID NO: 1, 28, and 82. In some embodiments of the methods as disclosed herein, the VL comprises an amino acid sequence selected from SEQ ID NO: 5, 32, and 86.

In another aspect, this disclosure provides, a method of treating a disease, comprising administering to a subject in need thereof an effective amount of a cell comprising an anti-BCMA chimeric antigen receptor (CAR) comprising an antigen binding domain, wherein the antigen binding domain comprises an antibody, Fab, or an scFv comprising a heavy chain variable region (VH) and a light chain variable region (VL),
wherein the VH comprises a CDR1 comprising the amino acid sequence of SEQ ID NO: 2; a CDR2 comprising the amino acid sequence of SEQ ID NO: 3; and a CDR3 comprising the amino acid sequence of SEQ ID NO: 4; and
wherein the VL comprises a CDR1 comprising the amino acid sequence of SEQ ID NO: 6; a CDR2 comprising the amino acid sequence of SEQ ID NO: 7; and a CDR3 comprising the amino acid sequence of SEQ ID NO: 8.

In some embodiments of the methods as disclosed herein, the VH comprises the amino acid sequence of SEQ ID NO: 1, and the VL comprises the amino acid sequence of SEQ ID NO: 5.

In some embodiments of the methods as disclosed herein, the methods further comprise inhibiting cancer growth, inducing cancer regression, and/or prolonging survival of the subject.

In some embodiments of the methods as disclosed herein, the cell further comprises decreased expression or knock out of one or more endogenous regulatory factors. In some embodiments, the one or more endogenous regulatory factors are selected from cyclin-dependent kinase inhibitor 2A (CDKN2A), cyclin-dependent kinase inhibitor 2B (CDKN2B), and S-methyl-5′-thioadenosine phosphorylase (MTAP). In an embodiment, the cell has decreased expression or knock out of CDKN2A, CDKN2B, and MTAP.

In some embodiments of the methods as disclosed herein, the cell does not express phosphatase and tensin homolog (PTEN).

In some embodiments of the methods as disclosed herein, the cell further comprises a transgene encoding either B-cell lymphoma-extra large (Bcl-xL) or B-cell lymphoma 2 (Bcl-2).

In some embodiments of the methods as disclosed herein, the cell does not express of one or more endogenous immune related genes. In some embodiments, the endogenous immune related gene is beta-2 microglobulin (B2M), and/or T-cell receptor a constant (TRAC).

In some embodiments of the methods as disclosed herein, the cell does not express cluster of differentiation 38 (CD38).

In some embodiments of the methods as disclosed herein, the cell is an autologous cell.

In some embodiments of the methods as disclosed herein, the cell is an allogeneic cell.

In some embodiments of the methods as disclosed herein, the cell is selected from a T cell, a Natural Killer (NK) cell, a cytotoxic T lymphocyte (CTL), a tumor infiltrating lymphocyte, a regulatory T cell, a CD8+ T cell, a CD4+ T cell, a gamma-delta T cell, a mucosal associated invariant T (MAIT) T cell, a natural killer T (NKT) cell, and/or a combination thereof.

In some embodiments of the methods as disclosed herein, the disease is a cancer selected from multiple myeloma (MM), chronic lymphocytic leukemia, acute B-lymphoblastic leukemia, non-Hodgkin lymphoma (NHL), Hodgkin lymphoma, acute myeloid leukemia (AML), and acute lymphoblastic leukemia (ALL). In an embodiment, the cancer is multiple myeloma.

In some embodiments of the methods as disclosed herein, the disease is an autoimmune disease. In an embodiment, the autoimmune disease is lupus.

In an aspect, this disclosure provides a pharmaceutical composition comprising the isolated nucleic acid as disclosed herein, the anti-BCMA CAR as disclosed herein, the vector as disclosed herein, or the cell as disclosed herein, and a pharmaceutically acceptable excipient.

In some embodiments of the methods as disclosed herein, the methods comprise administering to the subject the isolated nucleic acid as disclosed herein, the anti-BCMA CAR of any one of claims as disclosed herein, the vector as disclosed herein, the cell as disclosed herein, or the pharmaceutical composition as disclosed herein. In some embodiments of the methods as disclosed herein, the disease is a cancer or an autoimmune disease. In some embodiments, the cancer is selected from multiple myeloma (MM), chronic lymphocytic leukemia, acute B-lymphoblastic leukemia, non-Hodgkin lymphoma (NHL), Hodgkin lymphoma, acute myeloid leukemia (AML), and acute lymphoblastic leukemia (ALL). In an embodiment, the cancer is multiple myeloma. In an embodiment, the autoimmune disease is lupus.

In an aspect, the disclosure provides for a use of the isolated nucleic acid as disclosed herein, the anti-BCMA CAR as disclosed herein, the vector as disclosed herein, the cell as disclosed herein, or the pharmaceutical composition as disclosed herein in the treatment of a disease in a subject in need thereof.

In some embodiments, the disease is a cancer or an autoimmune disease. In some embodiments, the cancer is selected from multiple myeloma (MM), chronic lymphocytic leukemia, acute B-lymphoblastic leukemia, non-Hodgkin lymphoma (NHL), Hodgkin lymphoma, acute myeloid leukemia (AML), and acute lymphoblastic leukemia (ALL). In an embodiment, the cancer is multiple myeloma. In an embodiment, the autoimmune disease is lupus.

In an aspect, this disclosure provides for the use of an engineered cell for the manufacture of a medicament for treating a disease in a patient, wherein the engineered cell comprises an anti-BCMA chimeric antigen receptor (CAR) comprising an antigen binding domain, wherein the antigen binding domain comprises an antibody, Fab, or an scFv comprising a heavy chain variable region (VH) and a light chain variable region (VL),
wherein the VH comprises a CDR1 comprising an amino acid sequence selected from SEQ ID NO: 2, 29, and 83; a CDR2 comprising an amino acid sequence selected from SEQ ID NO: 3, 30, and 84; and a CDR3 comprising an amino acid sequence selected from SEQ ID NO: 4, 31, and 85; and
wherein the VL comprises a CDR1 comprising an amino acid sequence selected from SEQ ID NO: 6, 33, and 87; a CDR2 comprising an amino acid sequence selected from SEQ ID NO: 7, 34, and 88; and a CDR3 comprising an amino acid sequence selected from SEQ ID NO: 8, 35, and 89.

In some embodiments of the use as disclosed herein, the VH comprises a CDR1 comprising the amino acid sequence of SEQ ID NO: 2; a CDR2 comprising the amino acid sequence of SEQ ID NO: 3; and a CDR3 comprising the amino acid sequence of SEQ ID NO: 4; and wherein the VL comprises a CDR1 comprising the amino acid sequence of SEQ ID NO: 6; a CDR2 comprising the amino acid sequence of SEQ ID NO: 7; and a CDR3 comprising the amino acid sequence of SEQ ID NO: 8.

In some embodiments of the use as disclosed herein, the VH comprises an amino acid sequence selected from SEQ ID NO: 1, 28, and 82. In some embodiments of the use as disclosed herein, the VL comprises an amino acid sequence selected from SEQ ID NO: 5, 32, and 86.

In some embodiments of the use as disclosed herein, the VH comprises the amino acid sequence of SEQ ID NO: 1, and the VL comprises the amino acid sequence of SEQ ID NO: 5.

In an embodiment of the use as disclosed herein, the CAR has an amino acid sequence as set forth in SEQ ID NO: 96.

In an embodiment of the use as disclosed herein, the CAR has an amino acid sequence as set forth in SEQ ID NO: 97.

In an embodiment of the use as disclosed herein, the CAR has an amino acid sequence as set forth in SEQ ID NO: 98.

In some embodiments of the use as disclosed herein, the use further comprising inhibiting cancer growth, inducing cancer regression, and/or prolonging survival of the subject.

In some embodiments of the use as disclosed herein, the engineered cell further comprises decreased expression or knock out of one or more endogenous regulatory factors. In some embodiments of the use as disclosed herein, the one or more endogenous regulatory factors are selected from cyclin-dependent kinase inhibitor 2A (CDKN2A), cyclin-dependent kinase inhibitor 2B (CDKN2B), and S-methyl-5′-thioadenosine phosphorylase (MTAP). In an embodiment, the engineered cell has decreased expression or knock out of CDKN2A, CDKN2B, and MTAP.

In some embodiments of the use as disclosed herein, the engineered cell does not express phosphatase and tensin homolog (PTEN).

In some embodiments of the use as disclosed herein, the engineered cell further comprises a transgene encoding either B-cell lymphoma-extra large (Bcl-xL) or B-cell lymphoma 2 (Bcl-2).

In some embodiments of the use as disclosed herein, the engineered cell does not express of one or more endogenous immune related genes. In some embodiments of the use as disclosed herein, the endogenous immune related gene is beta-2 microglobulin (B2M), and/or T-cell receptor a constant (TRAC).

In some embodiments of the use as disclosed herein, the engineered cell does not express cluster of differentiation 38 (CD38).

In some embodiments of the use as disclosed herein, the engineered cell is an autologous cell.

In some embodiments of the use as disclosed herein, the engineered cell is an allogeneic cell.

In some embodiments of the use as disclosed herein, the engineered cell is selected from a T cell, a Natural Killer (NK) cell, a cytotoxic T lymphocyte (CTL), a tumor infiltrating lymphocyte, a regulatory T cell, a CD8+ T cell, a CD4+ T cell, a gamma-delta T cell, a mucosal associated invariant T (MAIT) T cell, a natural killer T (NKT) cell, and/or a combination thereof.

In some embodiments of the use as disclosed herein, the disease is a cancer selected from multiple myeloma (MM), chronic lymphocytic leukemia, acute B-lymphoblastic leukemia, non-Hodgkin lymphoma (NHL), Hodgkin lymphoma, acute myeloid leukemia (AML), and acute lymphoblastic leukemia (ALL). In some embodiments of the use as disclosed herein, the cancer is multiple myeloma. In some embodiments of the use as disclosed herein, the disease is an autoimmune disease. In some embodiments of the use as disclosed herein, the autoimmune disease is lupus.

In an aspect, this disclosure provides for an engineered cell for the manufacture of a medicament for treating a disease in a patient, wherein the engineered cell comprises an anti-BCMA chimeric antigen receptor (CAR) comprising an antigen binding domain, wherein the antigen binding domain comprises an antibody, Fab, or an scFv comprising a heavy chain variable region (VH) and a light chain variable region (VL),
wherein the VH comprises a CDR1 comprising an amino acid sequence selected from SEQ ID NO: 2, 29, and 83; a CDR2 comprising an amino acid sequence selected from SEQ ID NO: 3, 30, and 84; and a CDR3 comprising an amino acid sequence selected from SEQ ID NO: 4, 31, and 85; and
wherein the VL comprises a CDR1 comprising an amino acid sequence selected from SEQ ID NO: 6, 33, and 87; a CDR2 comprising an amino acid sequence selected from SEQ ID NO: 7, 34, and 88; and a CDR3 comprising an amino acid sequence selected from SEQ ID NO: 8, 35, and 89.

In some embodiments of the engineered cells as disclosed herein, the VH comprises a CDR1 comprising the amino acid sequence of SEQ ID NO: 2; a CDR2 comprising the amino acid sequence of SEQ ID NO: 3; and a CDR3 comprising the amino acid sequence of SEQ ID NO: 4; and wherein the VL comprises a CDR1 comprising the amino acid sequence of SEQ ID NO: 6; a CDR2 comprising the amino acid sequence of SEQ ID NO: 7; and a CDR3 comprising the amino acid sequence of SEQ ID NO: 8.

In some embodiments of the engineered cells as disclosed herein, the VH comprises an amino acid sequence selected from SEQ ID NO: 1, 28, and 82. In some embodiments of the engineered cells as disclosed herein, the VL comprises an amino acid sequence selected from SEQ ID NO: 5, 32, and 86.

In an embodiment of the engineered cells as disclosed herein, the VH comprises the amino acid sequence of SEQ ID NO: 1, and the VL comprises the amino acid sequence of SEQ ID NO: 5.

In an embodiment of the engineered cells as disclosed herein, the CAR has an amino acid sequence as set forth in SEQ ID NO: 96.

In an embodiment of the engineered cells as disclosed herein, the CAR has an amino acid sequence as set forth in SEQ ID NO: 97.

In an embodiment of the engineered cells as disclosed herein, the CAR has an amino acid sequence as set forth in SEQ ID NO: 98.

In some embodiments of the engineered cells as disclosed herein, the cells further comprises inhibiting cancer growth, inducing cancer regression, and/or prolonging survival of the subject.

In some embodiments of the engineered cells as disclosed herein, the engineered cell further comprises decreased expression or knock out of one or more endogenous regulatory factors. In some embodiments of the engineered cells as disclosed herein, the one or more endogenous regulatory factors are selected from cyclin-dependent kinase inhibitor 2A (CDKN2A), cyclin-dependent kinase inhibitor 2B (CDKN2B), and S-methyl-5′-thioadenosine phosphorylase (MTAP). In an embodiment, the engineered cell has decreased expression or knock out of CDKN2A, CDKN2B, and MTAP.

In some embodiments of the engineered cells as disclosed herein, the engineered cell does not express phosphatase and tensin homolog (PTEN).

In some embodiments of the engineered cells as disclosed herein, the engineered cell further comprises a transgene encoding either B-cell lymphoma-extra large (Bcl-xL) or B-cell lymphoma 2 (Bcl-2).

In some embodiments of the engineered cells as disclosed herein, the engineered cell does not express of one or more endogenous immune related genes. In some embodiments of the engineered cells as disclosed herein, the endogenous immune related gene is beta-2 microglobulin (B2M), and/or T-cell receptor a constant (TRAC).

In some embodiments of the engineered cells as disclosed herein, the engineered cell does not express cluster of differentiation 38 (CD38).

In some embodiments of the engineered cells as disclosed herein, the engineered cell is an autologous cell.

In some embodiments of the engineered cells as disclosed herein, the engineered cell is an allogeneic cell.

In some embodiments of the engineered cells as disclosed herein, the engineered cell is selected from a T cell, a Natural Killer (NK) cell, a cytotoxic T lymphocyte (CTL), a tumor infiltrating lymphocyte, a regulatory T cell, a CD8+ T cell, a CD4+ T cell, a gamma-delta T cell, a mucosal associated invariant T (MAIT) T cell, a natural killer T (NKT) cell, and/or a combination thereof.

In some embodiments of the engineered cells as disclosed herein, the disease is a cancer selected from multiple myeloma (MM), chronic lymphocytic leukemia, acute B-lymphoblastic leukemia, non-Hodgkin lymphoma (NHL), Hodgkin lymphoma, acute myeloid leukemia (AML), and acute lymphoblastic leukemia (ALL). In an embodiment, the cancer is multiple myeloma. In some embodiments, the disease is an autoimmune disease. In an embodiment, the autoimmune disease is lupus.

These and other features and advantages of the present disclosure will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The accompanying drawings are included to provide a further understanding of the methods and compositions of the disclosure. The drawings illustrate one or more embodiment(s) of the disclosure, and together with the description serve to explain the principles and operation of the disclosure.

FIG. 1 shows surface plasmon resonance binding of anti-BCMA scFv-Fcs to soluble BCMA protein. Mean values of ka, kd, and KD are reported.

FIG. 2 shows membrane proteome array for anti-BCMA scFv-Fes specificity to BCMA. Anti-BCMA scFv-Fcs were used in a cell-based membrane proteome array of >6000 membrane proteins to look at off-target binding. FIG. 2 illustrates the scFvs used for anti-BCMA CAR-T are specific for BCMA.

FIG. 3 shows anti-BCMA CAR staining of primary T cells. FIG. 3 illustrates that CARs successfully express on the surface of T cells. Staining is representative of 5 different donors.

FIG. 4 shows BCMA CAR-T binding curves. FIG. 4 illustrates binding properties of each anti-BCMA CAR-T clone for soluble BCMA antigen.

FIG. 5 shows anti-BCMA CAR-T expansion. FIG. 5 illustrates differences in growth kinetics and length of expansion of anti-BCMA CAR-T clones in primary T cells with IL-2 containing media.

FIGS. 6A and 6B show in vitro cytotoxicity of multiple myeloma cell lines. FIG. 6A and FIG. 6B illustrate BCMA CAR-T clone cytotoxicity across spectrum of multiple myeloma (MM) cell lines and compared to two clinical benchmarks. FIG. 6A is a heat map with average % cytotoxicity of anti-BCMA CAR-T clones from 4 donors across different MM cell lines at E:T ratio of 1:2. FIG. 6B shows bar graphs with average % cytotoxicity and standard deviation from 4 donors.

FIG. 7 shows effector cytokine production from anti-BCMA CAR-T clones. FIG. 7 illustrates representative cytokine production from anti-BCMA CAR-T cells when challenged with BCMA expressing huh7 engineered cell line.

FIG. 8 shows resistance to soluble BCMA. FIG. 8 illustrates differences in % cytolysis observed at 40 hrs post co-culture with BCMA expressing huh7 cells in the presence or absence of soluble BCMA protein (sBCMA).

FIG. 9 shows effector cytokine production in the presence of soluble BCMA. FIG. 9 illustrates the effects of soluble BCMA on CAR-T production of IFNy and IL-2 effector cytokines.

FIGS. 10A and 10B show persistence and expansion of CAR-T cells following repeat antigen stimulation in the presence or absence of soluble BCMA. FIG. 10A illustrates the anti-BCMA CAR-T cell expansion following repeated antigen stimulation (JJN3 co-culture) over the course of 12 days. During this time, % control of tumor cells was used as a measure of T cell function and persistence. FIG. 10B similarly illustrates antigen-dependent CAR-T cell expansion (JJN3 co-culture) in the presence of soluble antigen. % cytolysis of target cells highlights the impact of soluble BCMA protein on function and persistence of anti-BCMA CAR-T clones.

FIG. 11 shows in vivo tumor control of anti-BCMA CAR-T clones and clinical benchmarks. FIG. 11 illustrates CAR-T functional efficacy in vivo compared to clinical benchmarks at both high and low doses of CAR-T cells in a disseminated MM model (MM1.S).

FIG. 12 shows serum cytokine levels from in vivo tumor challenge model. FIG. 12 demonstrates CAR-T function at two doses via effector cytokine production following in vivo MM1.S tumor challenge.

FIG. 13 shows BCMA binding curves of BCMA CAR-T_{REX}. FIG. 13 illustrates BCMA binding properties of the 7A8.11 CAR when expressed in the CAR-T_{REX} chassis and benchmarked to primary CAR-T and clinical products.

FIG. 14 shows in vitro cytotoxicity of multiple myeloma cell lines. FIG. 14 illustrates BCMA CAR-T_{REX} cytotoxicity across a spectrum of multiple myeloma cell lines and compared to primary T cells and two clinical benchmarks.

FIG. 15 shows the effector cytokine profile from BCMA CAR-T_{REX} cells compared to primary T cells. FIG. 15 illustrates representative effector cytokine production from BCMA CAR-T_{REX} cells when encountering multiple myeloma target cell line JJN3 at E:T ratio of 1:1. BCMA CAR-T_{REX} demonstrate between 2-15% effector cytokine production compared to primary T cells of the same CAR and less than 5% of the clinical comparators suggesting a potentially safer cytokine profile.

FIG. 16 shows in vivo tumor control of MM1.S cells by BCMA CAR-T_{REX} compared to clinical benchmarks. FIG. 16 illustrates comparable or improved tumor clearing kinetics of BCMA CAR-T_{REX} cells relative to primary T cells of the same CAR and clinical benchmarks.

FIG. 17A-17C show that Daratumumab (Dara) treatment protects anti-BCMA-T_{REX} cell numbers and remaining T_{REX} cells are functional. After 5 hours, co-cultures were assessed by flow cytometry to quantify NK cell and anti-BCMA-T_{REX} cell numbers, demonstrating Dara-mediated protection of anti-BCMA-T_{REX} cell numbers (FIG. 17A). Cells were further progressed through two rounds of serial kill of JJN3 target cells at indicated E:T ratios, with % cytolysis of tumor cells measured by a luciferase assay (FIG. 17B); or a single round of cell killing with a BCMA-ectopically expressing SNU-182 adherent cell line, with tumor cell killing evaluated by Xcelligence (FIG. 17C).

FIG. 18 shows that BCMA CAR-T_{REX} cells effectively clear tumor from the bone marrow even when administered immediately following cryo-recovery. Cryo-recovered and immediately administered BCMA CAR-T_{REX} cells showed comparable tumor clearance and murine bone marrow restoration as other groups.

FIG. 19 shows that BCMA CAR-T_{REX} cells demonstrate deep in vivo tumor clearance when dosed immediately following cryo-recovery. NSG mice were inoculated with MM1S-luciferase tumor cells, and 3 days later primary BCMA CAR-T cells from two donors or BCMA CAR-T_{REX} cells (7A8.11) were administered immediately following cryo-recovery at indicated doses.

FIG. 20 shows the characterization of BCMA expression and B cell subsets in blood from healthy and SLE donors. Healthy and SLE donor PBMCs were isolated from fresh whole blood and were stained with antibodies. Cells shown are lymphocytes defined by FSC/SSC, live cells, dump gate negative, CD20+, and CD19+/−. DN2 cells (IgD−, CD27−), Memory B cells (CD27+IgD−), Naïve B cells (IgD+CD27−), NCSM (non-class switched memory, IgD+CD27+), plasma cells (CD19^{low/negative}CD138+CD38^{low}) and plasma blasts (CD19^{low/negative}CD138-CD38+CD27+).

FIG. 21 shows that BCMA CAR-T cells deplete healthy human plasma cells to a similar extent as MM1S (BCMA+) tumour cells. Primary plasmablasts, plasma cells and multiple myeloma MM1S cells were co-cultured with BCMA CAR-T cells or untransduced T cells at 1:1, 1:2, 1:4 effector:target ratios for 24 hours and then stained and assessed by flow cytometry. T-cells were gated as live, dump positive. Plasma cells were pre-gated on live, dump negative, CD19+, CD20 low, CD38+, CD27+ (plasma cells+plasmablasts).

FIG. 22 shows that in vitro differentiated plasmablasts from SLE and healthy donors show a dose dependent depletion (E:T) by BCMA CART-cells. Naïve B-cells from frozen healthy donors and SLE patients were isolated and plated utilizing a proprietary mix of cytokines to drive B-cell differentiation. After 5 days of differentiation, the differentiated B-cells were co-cultured with BCMA CAR-T cells or untransduced T cells at 1:1, 1:2, 1:4, 1:8, 1:16, and 1:32 effector:target ratios for 24 hours, then stained and assessed by flow cytometry. Percent depletion was calculated by 1−(% plasmablasts in experimental well/% average plasmablasts in stim only wells)*100.

FIG. 23 shows that BCMA targeting CAR-T cells reduced BCMA+ cells in xenogeneic model of graft versus host disease. Whole blood was collected for FACS analysis upon study termination on Day 12 post-engraftment. Top panel shows representative FACS histograms of splenic CD27+ memory B cells expressing BCMA. The bottom panel graphs show the percent BCMA+ of CD27+ memory B cells in the spleen, blood and bone marrow. Mean+/−standard error are shown for each treatment group. To determine statistically significant differences, one-way ANOVAs with Tukey's multiple comparisons tests were performed; *P<0.05, ****P<0.0001.

FIG. 24 shows serum cytokines/cytolytic granzymes were increased with BCMA CAR-T treatment. Serum collected from PBS, UTT and BCMA CAR-T treated mice was collected on Day 12 post-engraftment, and serum levels of IFN- [symbol] , GM-CSF, TNF-α, IL-2, Granzyme A and Granzyme B were assessed via ELISA. Shown are the mean+/−standard error for each cytokine from each treatment group. To determine statistically significant differences, one-way ANOVAs with Šídak's multiple comparisons tests were performed; *P<0.05, **P<0.01.

Skilled artisans will appreciate that elements in the Figures are illustrated for simplicity and clarity and have not necessarily been drawn to scale. For example, the dimensions of some of the elements in the Figures can be exaggerated relative to other elements to help improve understanding of the embodiment(s) of the present disclosure.

### DETAILED DESCRIPTION

Unless defined otherwise, all technical and scientific terms used herein have the meaning commonly understood by a person skilled in the art to which this disclosure belongs. The following references provide one of skill with a general definition of many of the terms used in this disclosure: Singleton, et al., Dictionary of Microbiology and Molecular Biology (2nd ed. 1994); The Cambridge Dictionary of Science and Technology (Walker ed., 1988); The Glossary of Genetics, 5th Ed., R. Rieger, et al. (eds.), Springer Verlag (1991); and Hale & Marham, The Harper Collins Dictionary of Biology (1991). As used herein, the following terms have the meanings ascribed to them below, unless specified otherwise.

As used herein, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps. Any of the terms “comprising,” “consisting essentially of,” and “consisting of” may be replaced with either of the other two terms, while retaining their ordinary meanings.

As used herein, the singular forms “a,” “an,” and “the” include plural referents unless the context clearly indicates otherwise.

Percentages disclosed herein can vary in amount by +10, 20, or 30% from values disclosed and remain within the scope of the contemplated disclosure.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

As used herein, ranges and amounts can be expressed as “about” a particular value or range. The term “about” also includes the exact amount. For example, “about 5%” means “about 5%” and also “5%.” The term “about” can also refer to +10% of a given value or range of values. Therefore, about 5% also means 4.5%-5.5%, for example. Additionally, “about” or “comprising essentially of” can mean a range of up to +10%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition. Unless otherwise clear from context, all numerical values provided herein are modified by the term “about.”

As described herein, any concentration range, percentage range, ratio range or integer range is to be understood to include the value of any integer within the recited range and, when appropriate, fractions thereof (such as one tenth and one hundredth of an integer), unless otherwise indicated.

Units, prefixes, and symbols are denoted in their Système International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

As used herein, the terms “or” and “and/or” can describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

As used herein, the term “polypeptide” refers to a molecule composed of monomers (amino acids) linearly linked by amide bonds (also known as peptide bonds). The term “polypeptide” refers to any chain or chains of two or more amino acids. Thus, peptides, dipeptides, tripeptides, oligopeptides, “protein,” “amino acid chain,” or any other term used to refer to a chain or chains of two or more amino acids are included within the definition of “polypeptide,” and the term “polypeptide” can be used instead of, or interchangeably with any of these terms.

A “protein” as used herein can refer to a single polypeptide, i.e., a single amino acid chain as defined above, but can also refer to two or more polypeptides that are associated, e.g., by disulfide bonds, hydrogen bonds, or hydrophobic interactions, to produce a multimeric protein.

An “isolated” substance, e.g., isolated nucleic acid, is a substance that is not in its natural milieu, though it is not necessarily purified. For example, an isolated nucleic acid is a nucleic acid that is not produced or situated in its native or natural environment, such as a cell. An isolated substance can have been separated, fractionated, or at least partially purified by any suitable technique.

As used herein, the terms “antibody” and “antigen-binding fragment thereof” refer to at least the minimal portion of an antibody which is capable of binding to a specified antigen which the antibody targets, e.g., at least some of the complementarity determining regions (CDRs) of the variable domain of a heavy chain (VH) and the variable domain of a light chain (VL) in the context of a typical antibody produced by a B cell. In some antibodies, e.g., naturally-occurring IgG antibodies, the heavy chain constant region is comprised of a hinge and three domains, CH1, CH2 and CH3. In some antibodies, e.g., naturally-occurring IgG antibodies, each light chain is comprised of a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system. A heavy chain can have the C-terminal lysine or not. Unless specified otherwise herein, the amino acids in the variable regions are numbered using the Kabat numbering system and those in the constant regions are numbered using the EU system.

Antibodies or antigen-binding fragments thereof can be or be derived from polyclonal, monoclonal, human, humanized, or chimeric antibodies, single chain antibodies, epitope-binding fragments, e.g., Fab, Fab′ and F(ab′)2, Fd, Fv, single-chain fragment variable (scFv), single-chain antibodies, VHH, vNAR, nanobody, (single-domain antibody), disulfide-linked Fvs (sdFvs), fragments comprising either a VL or VH domain alone or in conjunction with a portion of the opposite domain (e.g., a whole VL domain and a partial VH domain with one, two, or three CDRs), and fragments produced by a Fab expression library. ScFv molecules are known in the art and are described, e.g., in U.S. Pat. No. 5,892,019. Antibody molecules encompassed by this disclosure can be of or be derived from any type (e.g., IgG, IgE, IgM, IgD, IgA, and IgY), class (e.g., IgG1, IgG2, IgG3, IgG4, IgA1 and IgA2), or subclass of immunoglobulin molecule.

In certain aspects, this disclosure provides an anti-BCMA chimeric antigen receptor (CAR) comprising an antigen binding domain, wherein the antigen binding domain comprises an antibody, Fab, or an scFv comprising a heavy chain variable region (VH) and a light chain variable region (VL), wherein the VH comprises a CDR1 comprising an amino acid sequence selected from SEQ ID NO: 2, 11, 20, 29, 38, 47, 56, 65, 74, and 83; a CDR2 comprising an amino acid sequence selected from SEQ ID NO: 3, 12, 21, 30, 39, 48, 57, 66, 75, and 84; and a CDR3 comprising an amino acid sequence selected from SEQ ID NO: 4, 13, 22, 31, 40, 49, 58, 67, 76, and 85; and wherein the VL comprises a CDR1 comprising an amino acid sequence selected from SEQ ID NO: 6, 15, 24, 33, 42, 51, 60, 69, 78, and 87; a CDR2 comprising an amino acid sequence selected from SEQ ID NO: 7, 16, 25, 34, 43, 52, 61, 70, 79, and 88; and a CDR3 comprising an amino acid sequence selected from SEQ ID NO: 8, 17, 26, 35, 44, 53, 62, 71, 80, and 89. In certain embodiments, this disclosure provides an anti-BCMA chimeric antigen receptor (CAR) comprising an antigen binding domain, wherein the antigen binding domain comprises an antibody, Fab, or an scFv comprising a heavy chain variable region (VH) and a light chain variable region (VL), wherein the VH comprises an amino acid sequence selected from SEQ ID NO: 1, 10, 19, 28, 37, 46, 55, 64, 73, and 82; and wherein the VL comprises an amino acid sequence selected from SEQ ID NO: 5, 14, 23, 32, 41, 50, 59, 68, 77, and 86.

In one aspect, this disclosure provides an anti-BCMA CAR comprising or consisting of the amino acid sequence set forth as SEQ ID NO: 96. In another aspect, this disclosure provides an anti-BCMA CAR comprising or consisting of the amino acid sequence set forth as SEQ ID NO: 97. In yet another aspect, this disclosure provides an anti-BCMA CAR comprising or consisting of the amino acid sequence set forth as SEQ ID NO: 98.

As used herein, antibodies or antigen-binding fragments thereof also include “single-domain antibodies” which are antibodies whose complementary determining regions are part of a single domain polypeptide. Examples of single domain antibodies include heavy chain antibodies, antibodies naturally devoid of light chains, single domain antibodies derived from conventional four-chain antibodies, engineered or recombinant single-domain antibodies. Single domain antibodies may be derived from any species including, but not limited to mouse, human, camel, llama, goat, rabbit, and bovine. Single domain antibodies may be naturally occurring single domain antibodies known as heavy chain antibody devoid of light chains. In particular, Camelidae species, for example camel, dromedary, llama, alpaca and guanaco, produce heavy chain antibodies naturally devoid of light chain. The variable heavy chain of single-domain antibodies devoid of light chains are known as “VHH” or “nanobody”. Similar to conventional VH domains, VHHs contain four FRs and three CDRs. Nanobodies have advantages over conventional antibodies: they are smaller than IgG molecules, and as a consequence properly folded functional nanobodies can be produced by in vitro expression while achieving high yield. For example, VHH domains, Nanobodies and proteins/polypeptides containing the same can be produced using microbial fermentation and do not require the use of mammalian expression systems; VHH domains and nanobodies are relatively small (approximately 15 kDa, or 10 times smaller than a conventional IgG), and therefore show high (er) penetration into tissues (including but not limited to solid tumors and other dense tissues) than such conventional 4-chain antibodies and antigen-binding fragments thereof; VHH domains and nanobodies can show so-called cavity-binding properties (inter alia due to their extended CDR3 loop, compared to conventional VH domains) and can therefore also access targets and epitopes not accessable to conventional 4-chain antibodies and antigen-binding fragments thereof. Furthermore, nanobodies are very stable, and resistant to the action of proteases.

As used herein, “VHH domain” refers to variable domains present in naturally occurring heavy-chain antibodies, in order to distinguish them from the heavy chain variable domains that are present in conventional four-chain antibodies (referred to herein as “VH domains”) and from the light chain variable domains that are present in conventional four-chain antibodies (referred to herein as “VL domains”). In some embodiments, the recombinant polypeptides of the disclosure correspond to amino acid sequences of naturally occurring VHH domains, but that have been “humanized,” i.e., by replacing one or more amino acid residues in the amino acid sequence of the naturally occurring VHH sequence by one or more of the amino acid residues that occur at the corresponding positions in a VH domain from a conventional four-chain antibody from a human being. This can be performed in a manner known in the art.

In an embodiment, the disclosure provides recombinant polypeptide sequences, such as immunoglobulin sequences (in some embodiments, VHH antibody sequences) that are capable of binding to an envelope epitope of BCMA, wherein the immunoglobulin sequence comprises four framework regions (FR1, FR2, FR3, and FR4) and three complementarity determining regions (CDR1, CDR2, and CDR3), wherein:
a) CDR1 is the amino acid sequence of SEQ ID NO: 2, 11, 20, 29, 38, 47, 56, 65, 74, and 83; or selected from the group consisting of amino acid sequences that have at least 85%, or at least 90%, or at least 95%, or at least 99% sequence identity with the amino acid sequence of SEQ ID NO: 2, 11, 20, 29, 38, 47, 56, 65, 74, and 83; or from the group consisting of amino acid sequences that have 2 or only 1 amino acid differences as compared to the amino acid sequence of SEQ ID No: 2, 11, 20, 29, 38, 47, 56, 65, 74, and 83;
b) CDR2 is the amino acid sequence of SEQ ID NO: 3, 12, 21, 30, 39, 48, 57, 66, 75, and 84; or selected from the group consisting of amino acid sequences that have at least 85%, or at least 90%, or at least 95%, or at least 99% sequence identity with the amino acid sequence of SEQ ID NO: 3, 12, 21, 30, 39, 48, 57, 66, 75, and 84; or from the group consisting of amino acid sequences that have 2 or only 1 amino acid differences as compared to the amino acid sequence of SEQ ID No: 3, 12, 21, 30, 39, 48, 57, 66, 75, and 84;
c) CDR3 is the amino acid sequence of SEQ ID NO: 4, 13, 22, 31, 40, 49, 58, 67, 76, and 85; or selected from the group consisting of amino acid sequences that have at least 85%, or at least 90%, or at least 95%, or at least 99% sequence identity with the amino acid sequence of SEQ ID NO: 4, 13, 22, 31, 40, 49, 58, 67, 76, and 85; or from the group consisting of amino acid sequences that have 2 or only 1 amino acid differences as compared to the amino acid sequence of SEQ ID No: 4, 13, 22, 31, 40, 49, 58, 67, 76, and 85;
   and in which the framework sequences may be any suitable framework sequences, such as the framework sequences of a single-domain antibody and in particular of a VHH antibody.

B cell maturation antigen (BCMA; also referred to as BCM; CD269; and TNFRSF13A) is a member of the TNF-receptor superfamily. The receptor is expressed in mature B lymphocytes, and may be important for B cell development and autoimmune response. BCMA is also known as TNF receptor superfamily member 17 and has been shown to bind to the tumor necrosis factor superfamily, member 13b, and to lead to NF-kappaB and MAPK8/JNK activation. This receptor also binds to various TRAF family members, and thus may transduce signals for cell survival and proliferation.

The term “antigen-binding portion” of an antibody, as used herein, refers to one or more fragments of an antibody that retain the ability to specifically bind to an antigen (e.g., human BCMA). The antigen-binding function of an antibody can be performed by fragments of a full-length antibody. Examples of binding fragments encompassed within the term “antigen-binding portion” of an antibody, e.g., an anti-BCMA antibody described herein, include (i) a Fab fragment (fragment from papain cleavage) or a similar monovalent fragment consisting of the V_{L}, V_{H}, LC and CH1 domains; (ii) a F(ab′)2 fragment (fragment from pepsin cleavage) or a similar bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546), which consists of a V_{H} domain; (vi) an isolated complementarity determining region (CDR) and (vii) a combination of two or more isolated CDRs which can optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

The term “chimeric antigen receptor” or “CAR,” as used herein, refers to an engineered antigen-binding polypeptide, comprising an antigen-binding domain, a transmembrane domain, and one or more intracellular domains (e.g. costimulatory domains). In some embodiments, a CAR can optionally comprise a spacer domain and/or a flexible hinge domain to provide conformational freedom to facilitate binding to the target antigen on the target cell. In some embodiments, a CAR can optionally comprise an armoring domain comprising a nucleic acid sequence encoding an armoring molecule. Expression of a CAR on the surface of a cell, e.g., an immune cell, allows the cell to target and bind a particular antigen. In some embodiments, the CAR is expressed by an immune cell, e.g., a T cell. In some embodiments, the antigen binding domain comprises an Fab, Fab′, F(ab′)2, Fd, Fv, single-chain fragment variable (scFv), single chain antibody, VHH, vNAR, nanobody (single-domain antibody), or any combination thereof. In some embodiments, the transmembrane domain comprises a transmembrane domain selected from the transmembrane domain of CD4, CD8α, or CD28. In some embodiments, the one or more intracellular domains comprises a costimulatory domain or a portion thereof. In some embodiments, the intracellular domain comprises a costimulatory domain or a portion thereof. In some embodiments, the intracellular domain comprises a costimulatory domain of CD3z or variants thereof. For instance, the CD3z costimulatory domain variants may contain only 1 or 2 functional immunoreceptor tyrosine-based activation motifs (ITAMs) of the three ITAMs present in wild-type CD3z. In some embodiments, the intracellular domain comprises a costimulatory domain selected from the group consisting a CD3zeta costimulatory domain, CD28 costimulatory domain, a CD27 costimulatory domain, a 4-1BB costimulatory domain, an ICOS costimulatory domain, an OX-40 costimulatory domain, a GITR costimulatory domain, a CD2 costimulatory domain, an IL-2RB costimulatory domain, a MyD88/CD40 costimulatory domain, and any combination thereof. A CAR can further comprise a “hinge” or “spacer” domain. Non-limiting examples of hinge/spacer domains include immunoglobulin hinge/spacer domains, such as an IgG1 hinge domain, an IgG2 hinge domain, an IgG3 hinge domain, an IgG4 hinge domain, an IgG4P hinge domain (an IgG4 hinge domain comprising a S241P mutation), a CD8a hinge domain, or a CD28 hinge domain. In an embodiment, a CAR comprises a hinge comprising the sequence of SEQ ID NO:93.

As used herein, the term “polynucleotide” includes a singular nucleic acid as well as multiple nucleic acids, and refers to an isolated nucleic acid molecule or construct, e.g., messenger RNA (mRNA) or plasmid DNA (pDNA). The term “nucleic acid” includes any nucleic acid type, such as DNA or RNA. “Conservative amino acid substitutions” refer to substitutions of an amino acid residue with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). In some embodiments, a predicted nonessential amino acid residue in a BCMA-binding moiety (e.g., an anti-BCMA CAR or antibody) is replaced with another amino acid residue from the same side chain family.

The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (freely available), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48): 444-453 (1970)) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences described herein can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, word length=12 to obtain nucleotide sequences homologous to the nucleic acid molecules described herein. BLAST protein searches can be performed with the XBLAST program, score=50, word length=3 to obtain amino acid sequences homologous to the protein molecules described herein. To obtain gapped alignments for comparison purposes, Gapped BLAST can be utilized as described in Altschul et al., (1997) Nucleic Acids Res. 25 (17): 3389-3402. When utilizing BLAST and Gapped BLAST programs, the default parameters of the respective programs (e.g., XBLAST and NBLAST) can be used.

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” can be used interchangeably as the plasmid is the most commonly used form of vector. However, also included are other forms of expression vectors, such as viral vectors (e.g., lentiviral vectors, replication defective retroviruses, adenoviruses and adeno-associated viruses), or transposons (e.g. DNA transposons or retrotransposons) which serve equivalent functions. In certain embodiments, the CARs and/or antibodies or antigen binding fragments thereof are encompassed by and/or delivered to a cell and or patient using a virus, a lentivirus, an adenovirus, a retrovirus, an adeno-associated virus (AAV), a transposon, a DNA vector, a mRNA, a lipid nanoparticle (LNP), or a CRISPR-Cas System. In an embodiment, lentiviral vectors are used.

As used herein, the term “vector” can refer to a nucleic acid molecule as introduced into a host cell, thereby producing a transformed host cell. A vector can include nucleic acid sequences that permits it to replicate in a host cell, such as an origin of replication. A vector can also include one or more selectable marker gene(s) and other genetic elements known in the art. Specific types of vector envisioned here can be associated with or incorporated into viruses to facilitate cell transformation.

A “transformed” cell, or a “host” cell, is a cell into which a nucleic acid molecule has been introduced by molecular biology techniques. All techniques by which a nucleic acid molecule can be introduced into such a cell, including transfection with viral vectors, transformation with plasmid vectors, and introduction of naked DNA by electroporation, lipofection, and particle gun acceleration are contemplated herein. In certain embodiments, cells are transformed by one or more techniques using a virus, a lentivirus, an adenovirus, a retrovirus, an adeno-associated virus (AAV), a transposon, a DNA vector, a mRNA, a lipid nanoparticle (LNP), and a CRISPR-Cas System.

As used herein, the term “affinity” refers to a measure of the strength of the binding of a antigen or target (such as an epitope) to its cognate binding domain (such as a paratope). As used herein, the term “avidity” refers to the overall stability of the complex between a population of epitopes and paratopes (i.e., antigens and antigen binding domains).

The term “epitope” refers to a site on an antigen (e.g., BCMA) to which a chimeric antigen receptor, immunoglobulin, or antibody specifically binds, e.g., as defined by the specific method used to identify it. Epitopes can be formed both from contiguous amino acids (usually a linear epitope) or non-contiguous amino acids juxtaposed by tertiary folding of a protein (usually a conformational epitope). Epitopes formed from contiguous amino acids are typically, but not always, retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation.

“Immunotherapy” refers to the treatment of a subject afflicted with, or at risk of contracting or suffering a recurrence of, a disease by a method comprising inducing, enhancing, suppressing or otherwise modifying the immune system or an immune response.

An “immune response” is as understood in the art, and generally refers to a biological response within a vertebrate against foreign agents or abnormal, e.g., cancerous cells, which response protects the organism against these agents and diseases caused by them. An immune response is mediated by the action of one or more cells of the immune system (for example, a T lymphocyte, B lymphocyte, natural killer (NK) cell, macrophage, cosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell, a Th cell, a CD4+ cell, a CD8+ T cell, or a Treg cell, or activation or inhibition of any other cell of the immune system, e.g., NK cell.

As used herein, the terms “treat,” “treatment,” or “treatment of” when used in the context of treating cancer refer to reducing disease pathology, reducing or eliminating disease symptoms, promoting increased survival rates, and/or reducing discomfort. For example, treating can refer to the ability of a therapy when administered to a subject, to reduce disease symptoms, signs, or causes. Treating also refers to mitigating or decreasing at least one clinical symptom and/or inhibition or delay in the progression of the condition and/or prevention or delay of the onset of a disease or illness.

As used herein, the terms “subject,” “individual,” or “patient,” refer to any subject, particularly a mammalian subject, for whom diagnosis, prognosis, or therapy is desired. Mammalian subjects include, for example, humans, non-human primates, dogs, cats, guinea pigs, rabbits, rats, mice, horses, cattle, bears, and so on.

As used herein, the term an “effective amount” or a “therapeutically effective amount” of an administered therapeutic substance, such as a CAR T cell, is an amount sufficient to carry out a specifically stated or intended purpose, such as treating or treatment of cancer. An “effective amount” can be determined empirically in relation to the stated purpose. In certain embodiments, a therapeutically effective amount can refer to the number of cells administered to a subject in need of treatment. The number of cells per dose, the number of doses, and frequency of dosing will depend on various parameters such as the patient's age, weight, clinical assessment, disease type, cancer type, tumor type, tumor burden, and/or other factors, including the judgment of the attending physician.

The terms “T cell” or “T lymphocyte” are art-recognized and are intended to include thymocytes, naive T lymphocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4+ T cell) CD4+ T cell, a cytotoxic T cell (CTL; CD8+ T cell), a tumor infiltrating cytotoxic T cell (TIL; CD8+ T cell), CD4+CD8+ T cell, CD4 CD8-T cell, or any other subset of T cells. Other illustrative populations of T cells suitable for use in particular embodiments include naive T cells and memory T cells.

As used herein, the term “proliferation” refers to an increase in cell division, either symmetric or asymmetric division of cells. In particular embodiments, “proliferation” refers to the symmetric or asymmetric division of T cells. “Increased proliferation” occurs when there is an increase in the number of cells in a treated sample compared to cells in a non-treated sample.

The term “expanding” in the method of the disclosure refers to the process of increasing the number of cells in a cell culture. In the expanding step, cells are fed and culture media is replaced at regular intervals, in one embodiment according to a feed regimen. The specific timings and amounts of media added in a particular feed regimen will depend on the cell number and the levels of metabolites in the culture.

As used herein, the term “differentiation” refers to a method of decreasing the potency or proliferation of a cell or moving the cell to a more developmentally restricted state. In particular embodiments, differentiated T cells acquire immune effector cell functions.

An “immune effector cell,” is any cell of the immune system that has one or more effector functions (e.g., cytotoxic cell killing activity, secretion of cytokines, induction of ADCC and/or CDC). The illustrative immune effector cells contemplated herein are NK cells or T lymphocytes, in particular cytotoxic T cells (CTLs; CD8+ T cells), TILs, and helper T cells (HTLs; CD4+ T cells).

“Modified T cells” refer to T cells that have been modified by the introduction of a polynucleotide encoding an engineered CAR contemplated herein. Modified T cells include both genetic and non-genetic modifications (e.g., episomal or extrachromosomal).

As used herein, the term “genetically engineered” or “genetically modified” refers to the addition of extra genetic material in the form of DNA or RNA into the total genetic material in a cell.

The terms, “genetically modified cells,” “modified cells,” and, “redirected cells,” are used interchangeably.

The acronym “SMART” (Shorty-Manipulated Auto-Replicating T-Cells) refers to a shortened T cell manufacture and expansion process wherein the cells are cultured in the presence of IL-21 (and optionally IL-2).

The acronym “TNT” (Traditionally Nurtured T-Cells) refers to a traditional T-cell expansion process which does not employ IL-21, and typically comprises a cell culture for more than 7 days and/or typically comprises the use of IL-2.

The term “stimulation” refers to a primary response induced by binding of a stimulatory molecule (e.g., a TCR/CD3 complex) with its cognate ligand thereby mediating a signal transduction event including, but not limited to, signal transduction via the TCR/CD3 complex.

A “stimulatory molecule,” refers to a molecule on a T cell that specifically binds with a cognate stimulatory ligand.

A “stimulatory ligand,” as used herein, means a ligand that when present on an antigen presenting cell (e.g., an APC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands include, but are not limited to CD3 ligands, e.g., an anti-CD3 antibody and CD2 ligands, e.g., anti-CD2 antibody, and peptides, e.g., CMV, HPV, EBV peptides.

The term, “activation” refers to the state of a T cell that has been sufficiently stimulated to induce detectable cellular proliferation. In particular embodiments, activation can also be associated with induced cytokine production, and detectable effector functions. The term “activated T cells” refers to, among other things, T cells that are proliferating. Signals generated through the TCR alone are insufficient for full activation of the T cell and one or more secondary or costimulatory signals are also required. Thus, T cell activation comprises a primary stimulation signal through the TCR/CD3 complex and one or more secondary costimulatory signals. Costimulation can be evidenced by proliferation and/or cytokine production by T cells that have received a primary activation signal, such as stimulation through the CD3/TCR complex or through CD2.

A “costimulatory signal,” refers to a signal, which in combination with a primary signal, such as TCR/CD3 ligation, leads to T cell proliferation, cytokine production, and/or upregulation or downregulation of particular molecules (e.g., CD28).

A “costimulatory ligand,” refers to a molecule that binds a costimulatory molecule. A costimulatory ligand may be soluble or provided on a surface. A “costimulatory molecule” refers to the cognate binding partner on a T cell that specifically binds with a costimulatory ligand (e.g., anti-CD28 antibody).

“Autologous,” as used herein, refers to cells from the same subject. In some embodiments, the cells of the disclosure are autologous.

“Allogeneic,” as used herein, refers to cells of the same species that differ genetically to the cell in comparison. In some embodiments, the cells of the disclosure are allogeneic.

“Syngeneic,” as used herein, refers to cells of a different subject that are genetically identical to the cell in comparison. In some embodiments, the cells of the disclosure are syngeneic.

“Xenogeneic,” as used herein, refers to cells of a different species to the cell in comparison. In some embodiments, the cells of the disclosure are xenogeneic.

As used herein, the terms “individual” and “subject” are often used interchangeably and refer to any animal that exhibits a symptom of a disease that can be treated with the gene therapy vectors, cell-based therapeutics, and methods disclosed elsewhere herein. Suitable subjects (e.g., patients) include laboratory animals (such as mouse, rat, rabbit, or guinea pig), farm animals, and domestic animals or pets (such as a cat or dog). Non-human primates and, preferably, human patients, are included. Typical subjects include human patients that have a cancer, have been diagnosed with a cancer, or are at risk or having a cancer.

By “enhance” or “promote,” or “increase” or “expand” refers generally to the ability of a composition contemplated herein to produce, elicit, or cause a greater physiological response (i.e., downstream effects) compared to the response caused by either vehicle or a control molecule/composition. A measurable physiological response may include an increase in T cell expansion, activation, persistence, and/or an increase in cancer cell death killing ability, among others apparent from the understanding in the art and the description herein. An “increased” or “enhanced” amount is typically a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by vehicle or a control composition.

By “decrease” or “lower,” or “lessen,” or “reduce,” or “abate” refers generally to the ability of composition contemplated herein to produce, elicit, or cause a lesser physiological response (i.e., downstream effects) compared to the response caused by either vehicle or a control molecule/composition. A “decrease” or “reduced” amount is typically a “statistically significant” amount, and may include an decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response (reference response) produced by vehicle, a control composition, or the response in a particular cell lineage.

By “maintain,” or “preserve,” or “maintenance,” or “no change,” or “no substantial change,” or “no substantial decrease” refers generally to the ability of a composition contemplated herein to produce, elicit, or cause a lesser physiological response (i.e., downstream effects) in a cell, as compared to the response caused by either vehicle, a control molecule/composition, or the response in a particular cell lineage. A comparable response is one that is not significantly different or measurably different from the reference response.

### Overview

In some aspects, the present disclosure is directed to compositions and methods for treating a disease using chimeric antigen receptor (CAR) cell therapy. More particularly, the present disclosure concerns CAR cell therapies in which the transformed cells, such as T cells or NK cells, express CARs that target BCMA. Still further, the CAR constructs, transformed cells expressing the constructs, and the therapies utilizing the transformed cells disclosed herein can provide robust treatments against cancer, autoimmune disease, or other diseases expressing BCMA.

Without wishing to be bound by theory, BCMA is believed to be a viable disease target across multiple modalities, and it is believed that BCMA is a promising target for CAR cell therapy.

### CAR Construct Design

CAR constructs of the present disclosure can have several components, many of which can be selected based upon a desired or refined function of the resultant CAR construct. In addition to an antigen binding domain, CAR constructs can have a spacer domain, a hinge domain, a signal peptide domain, a transmembrane domain, and one or more intracellular domains (for example, one or more costimulatory domains). In some embodiments, a CAR can optionally comprise an armoring domain comprising a nucleic acid sequence encoding an armoring molecule. Selection of one component over another (i.e., selection of a specific costimulatory domain from one receptor versus a costimulatory domain from a different receptor) can influence clinical efficacy and safety profiles.

### Antigen Binding Domain

Antigen binding domains contemplated herein can include antibodies or one or more antigen-binding fragments thereof. One contemplated CAR construct targeting BCMA comprises a single chain variable fragment (scFv) containing light and heavy chain variable regions from one or more antibodies specific for BCMA that are either directly linked together or linked together via a flexible linker (e.g., a repeat of G₄S having 1, 2, 3 or more repeats). In an embodiment, the linker comprises the sequence of SEQ ID NO:92.

The antigen binding domain of a CAR targeting BCMA as disclosed herein can vary in its binding affinity for the BCMA protein. The relationship between binding affinity and efficacy can be more nuanced in the context of CARs as compared with antibodies, for which higher affinity is typically desirable. For example, preclinical studies on a receptor tyrosine kinase-like orphan receptor 1 (ROR1)-CAR derived from a high-affinity scFv (with a dissociation constant of 0.56 nM) resulted in an increased therapeutic index when compared with a lower-affinity variant. Conversely, other examples have been reported that engineering the scFv for lower affinity improves the discrimination among cells with varying antigen density. This could be useful for improving the therapeutic specificity for antigens differentially expressed on tumor versus normal tissues.

A variety of methods can be used to ascertain the binding affinity of the antigen binding domain. In some embodiments, methodologies that exclude avidity effects can be used. Avidity effects involve multiple antigen-binding sites simultaneously interacting with multiple target epitopes, often in multimerized structures. Thus, avidity functionally represents the accumulated strength of multiple interactions. An example of a methodology that excludes avidity effects is any approach in which one or both of the interacting proteins is monomeric/monovalent since multiple simultaneous interactions are not possible if one or both partners contain only a single interaction site.

### Spacer Domain

A CAR construct of the present disclosure can have a spacer domain to provide conformational freedom to facilitate binding to the target antigen on the target cell. The optimal length of a spacer domain may depend on the proximity of the binding epitope to the target cell surface. For example, proximal epitopes can require longer spacers and distal epitopes can require shorter ones. Besides promoting binding of the CAR to the target antigen, achieving an optimal distance between a CAR cell and a cancer cell may also help to sterically occlude large inhibitory molecules from the immunological synapse formed between the CAR cell and the target cancer cell. A CAR targeting BCMA can have a long spacer, an intermediate spacer, or a short spacer. Long spacers can include a CH2CH3 domain (˜220 amino acids) of immunoglobulin G1 (IgG1) or IgG4 (either native or with modifications common in therapeutic antibodies, such as a S228P mutation), whereas the CH3 region can be used on its own to construct an intermediate spacer (˜120 amino acids). Shorter spacers can be derived from segments (<60 amino acids) of CD28, CD8α, CD3 or CD4. Short spacers can also be derived from the hinge regions of IgG molecules. These hinge regions may be derived from any IgG isotype and may or may not contain mutations common in therapeutic antibodies such as the S228P mutation mentioned above. For example, a hinge domain can comprise an IgG1 hinge domain or variants thereof, an IgG2 hinge domain or variants thereof, an IgG3 hinge domain or variants thereof, an IgG4 hinge domain or variants thereof, a CD8 hinge domain or variants thereof or a CD28 hinge domain or variants thereof.

### Hinge Domain

A CAR targeting BCMA can also have a hinge domain. The flexible hinge domain is a short peptide fragment that provides conformational freedom to facilitate binding to the target antigen on the tumor cell. It may be used alone or in conjunction with a spacer sequence. The terms “hinge” and “spacer” are often used interchangably—for example, IgG4 sequences can be considered both “hinge” and “spacer” sequences (i.e., hinge/spacer sequences). In some embodiments, a hinge domain can comprise an IgG1 hinge domain or variants thereof, an IgG2 hinge domain or variants thereof, an IgG3 hinge domain or variants thereof, an IgG4 hinge domain or variants thereof (in particular, an IgG4P hinge domain), a CD8 hinge domain or variants thereof or a CD28 hinge domain or variants thereof. In an embodiment, the hinge domain comprises the sequence of SEQ ID NO:93.

### Signal Peptide

A CAR targeting BCMA can further include a sequence comprising a signal peptide. Signal peptides function to prompt a cell to translocate the CAR to the cellular membrane. Examples include an IgG1 heavy chain signal polypeptide, Ig kappa or lambda light chain signal peptides, granulocyte-macrophage colony stimulating factor receptor 2 (GM-CSFR2 or CSFR2) signal peptide, a CD8a signal polypeptide, or a CD33 signal peptide. In an embodiment, the signale peptide comprises the sequence of SEQ ID NO:91.

### Transmembrane Domain

A CAR targeting BCMA can further include a sequence comprising a transmembrane domain. The transmembrane domain can include a hydrophobic a helix that spans the cell membrane. The properties of the transmembrane domain have not been as meticulously studied as other aspects of CAR constructs, but they can potentially affect CAR expression and association with endogenous membrane proteins. Transmembrane domains can be derived, for example, from CD3, CD4, CD8α, or CD28. Any transmembrane domain can be used in the compositions disclosed herein. In some embodiments, the transmembrane domain comprises a transmembrane domain selected from the transmembrane domain of CD3, CD4, CD8α, or CD28. In some embodiments, the transmembrane domain comprises a CD28 transmembrane domain. In an embodiment, the transmembrane domain comprises the sequence of SEQ ID NO:94.

### Intracellular Domain/Costimulatory Domain

A CAR targeting BCMA can further include one or more sequences that form an intracellular domain and/or a costimulatory domain (also sometimes referred to as a signaling domain). A costimulatory domain is a domain capable of potentiating or modulating the response of immune effector cells (i.e., capable of initiating the response of immune effector cells). In some embodiments, the costimulatory domains and/or signaling domains comprises a primary activation signal derived from the cytoplasmic domains of CD3ζ, which contains sequence motifs called immunoreceptor tyrosine-based activation motifs (ITAMs)). In certain embodiments an intracellular domain refers to the combination of a costimulatory domain (e.g. a costimulatory domain from 4-1BB or CD28) plus the primary activation signal of CD3ζ (CD3z or CD3zeta). Costimulatory domains can include sequences, for example, a costimulatory domain from one or more of CD28, 4-1BB, OX-40, ICOS, CD27, GITR, CD2, IL-2RB and MyD88/CD40. In certain embodiments, a costimulatory domain selected from CD28, 4-1BB, OX-40, ICOS, CD27, GITR, CD2, IL-2RB and MyD88/CD40 is combined with the primary activation signal of CD3 (CD3z or CD3zeta). In some embodiments, the costimulatory domain, can include variants of a costimulatory domain one or more of CD28, 4-1BB, OX-40, ICOS, CD27, GITR, CD2, IL-2Rβ and MyD88/CD40. In certain embodiments, a costimulatory domain variant is selected from a costimulatory domain variant of CD28, 4-1BB, OX-40, ICOS, CD27, GITR, CD2, IL-2RB and MyD88/CD40, and is combined with the primary activation signal of CD35 (CD3z or CD3zeta). In an embodiment, a CAR costimulatory domain can further include modifications to the CD32 domain. For example, the CD3z signaling domain variants may contain 1 or 2 functional immunoreceptor tyrosine-based activation motifs (ITAMs) of the three ITAMs present in wild-type CD3z. The choice of costimulatory domain influences the phenotype and metabolic signature of CAR cells. For example, CD28 costimulation yields a potent, yet short-lived, effector-like phenotype, with high levels of cytolytic capacity, interleukin-2 (IL-2) secretion, and glycolysis. By contrast, T cells modified with CARs bearing 4-1BB costimulatory domains tend to expand and persist longer in vivo, have increased oxidative metabolism, are less prone to exhaustion, and have an increased capacity to generate central memory T cells. In some embodiments, the intracellular signaling domain comprises a costimulatory domain or a portion thereof. In an embodiment, the an intracellular domain comprises the sequence of SEQ ID NO:95.

In some embodiments, the intracellular domain comprises a costimulatory domain selected from the group consisting of the intracellular domain of a CD28 costimulatory domain, a CD27 costimulatory domain, a 4-1BB costimulatory domain, an ICOS costimulatory domain, an OX-40 costimulatory domain, a GITR costimulatory domain, a CD2 costimulatory domain, an IL-2RB costimulatory domain, a MyD88/CD40 costimulatory domain, and any combination thereof. In some embodiments, the intracellular domain comprises a CD28 costimulatory domain. In some embodiments, the intracellular domain comprises a 4-1BB costimulatory domain. In some embodiments, the intracellular domain comprises a CD28 costimulatory domain in combination with CD3zeta. In some embodiments, the intracellular domain comprises a 4-1BB costimulatory domain in combination with CD3zeta. In an embodiment, the intracellular domain comprises the sequence of SEQ ID NO:95.

In certain embodiments, the intracellular domain comprises a costimulatory domain comprising a portion of the intracellular T cell receptor (TCR) signaling domain, CD3zeta (or CD3z; the CD3z signaling domain is also referred to herein as a “CD3z costimulatory domain”). In some embodiments, the CD3zeta comprises one or more modifications to the CD3z format. For example, CD3z signaling domain variants may contain 1 or 2 functional immunoreceptor tyrosine-based activation motifs (ITAMs) of the three ITAMs present in wild-type CD3z (e.g. 1XX, XIX, or X2X).

### Exemplary CAR

According to all aspects of the invention, the CAR may comprise or consist of the amino acid sequence set forth as SEQ ID NO: 96. According to all aspects of the invention, the CAR may comprise or consist of the amino acid sequence set forth as SEQ ID NO: 97. According to all aspects of the invention, the CAR may comprise or consist of the amino acid sequence set forth as SEQ ID NO: 98.

In certain aspects, this disclosure provides, an anti-BCMA chimeric antigen receptor (CAR) comprising an antigen binding domain, wherein the antigen binding domain comprises an antibody, Fab, or an scFv comprising a heavy chain variable region (VH) and a light chain variable region (VL), wherein the VH comprises a CDR1 comprising an amino acid sequence selected from SEQ ID NO: 2, 11, 20, 29, 38, 47, 56, 65, 74, and 83; a CDR2 comprising an amino acid sequence selected from SEQ ID NO: 3, 12, 21, 30, 39, 48, 57, 66, 75, and 84; and a CDR3 comprising an amino acid sequence selected from SEQ ID NO: 4, 13, 22, 31, 40, 49, 58, 67, 76, and 85; and wherein the VL comprises a CDR1 comprising an amino acid sequence selected from SEQ ID NO: 6, 15, 24, 33, 42, 51, 60, 69, 78, and 87; a CDR2 comprising an amino acid sequence selected from SEQ ID NO: 7, 16, 25, 34, 43, 52, 61, 70, 79, and 88; and a CDR3 comprising an amino acid sequence selected from SEQ ID NO: 8, 17, 26, 35, 44, 53, 62, 71, 80, and 89.

In certain embodiments, this disclosure provides an anti-BCMA chimeric antigen receptor (CAR) comprising an antigen binding domain, wherein the antigen binding domain comprises an antibody, Fab, or an scFv comprising a heavy chain variable region (VH) and a light chain variable region (VL), wherein the VH comprises an amino acid sequence selected from SEQ ID NO: 1, 10, 19, 28, 37, 46, 55, 64, 73, and 82; and wherein the VL comprises an amino acid sequence selected from SEQ ID NO: 5, 14, 23, 32, 41, 50, 59, 68, 77, and 86.

CAR constructs of the present disclosure can include some combination of the modular components described herein. For example, in some embodiments of the present disclosure, a CAR construct comprises a BCMA scFv antigen binding domain. In some embodiments of the present disclosure, a CAR construct comprises a CD33 signal peptide. In an embodiment, a CAR comprises a signal peptide comprising the sequence of SEQ ID NO: 91. In some embodiments, a CAR construct comprises an IgG4 hinge/spacer domain carrying an S241P mutation (IgG4P). In an embodiment, a CAR comprises a hinge domain comprising the sequence of SEQ ID NO:93. In some embodiments, a CAR construct comprises a CD28 transmembrane domain. In an embodiment, a CAR comprises a transmembrane domain comprising the sequence of SEQ ID NO:94.

Different costimulatory domains can be utilized in the CAR constructs of the present disclosure. In some embodiments, a CAR construct comprises a costimulatory domain comprising a signaling domain from the intracellular domain of CD3z (for example, a portion of the intracellular T cell receptor (TCR) signaling domain, CD3zeta (or CD3z) or a variant thereof). In some embodiments, a CAR construct comprises a CD28 costimulatory domain. In some embodiments, a CAR construct comprises a 4-1BB costimulatory domain. In some embodiments, a CAR construct comprises costimulatory domains from CD3z and CD28, as described herein. In some embodiments, a CAR construct comprises costimulatory domains from CD3z and 4-1BB, as described herein. In some embodiments, a CAR construct comprises costimulatory domains from all of CD3z, CD28, and 4-1BB, as described herein. In some embodiments, a CAR construct comprises costimulatory domains from ICOS, OX-40, and/or GITR. In an embodiment, a CAR comprises an intracellular domain comprising the sequence of SEQ ID NO:95.

### Cells

CAR-based cell therapies can be used with a variety of cell types, such as lymphocytes. Particular types of cells that can be used include T cells, Natural Killer (NK) cells, Natural Killer T (NKT) cells, Invariant Natural Killer T (INKT) cells, alpha beta T cells, gamma delta T cells, viral-specific T (VST) cells, cytotoxic T lymphocytes (CTLs), tumor infiltrating lymphocytes, and regulatory T cells (Tregs). In some embodiments, the cells are autologous. In certain embodiments, the cells are allogeneic. In other embodiments, the cells may be from a genetically similar, but non-identical donor (allogeneic).

In some embodiments, the population of cells can also include expanded populations, and/or the engineered T cells. In some embodiments, the population of cells can comprise total T cells, CD4-positive T cells, CD8-positive T cells, regulatory T cells, gamma-delta T cells, mucosal associated invariant T (MAIT) T cells, natural killer (NK) cells, or natural killer T (NKT) cells. T cells are broadly divided into cells expressing CD4 on their surface (also referred to as CD4-positive cells) and cells expressing CD8 on their surface (also referred to as CD8-positive cells).

In some embodiments, the T cells appropriate for use according to the methods provided herein are mononuclear lymphocytes derived from bone marrow (BM), peripheral blood (PB), or cord blood (CB) of a human donor. These cells could be collected directly from BM, PB, or CB or after mobilization or stimulation via administration of growth factors and/or cytokines such as granulocyte-colony stimulating factor (G-CSF) or granulocyte-macrophage colony-stimulating factor (GM-CSF) to allogeneic or autologous donors. Those skilled in the art would appreciate that there are many established protocols for isolating peripheral blood mononuclear cells (PBMC) from peripheral blood. Isolation of PBMC can be aided by density-gradient separation protocols, usually employing a density-gradient centrifugation technique using Ficoll®-Hypaque or Histopaque® for separating lymphocytes from other elements in the blood. Preferably, PBMC isolation is performed under sterile conditions. Isolation of PBMC can also use negative selection kits. Alternatively, cell clutriation methods may be employed to separate mononuclear cell populations. In some embodiments, the population of cells are human cells. In certain embodiments, the population of cells are human primary immune cells.

In some embodiments, the cell compositions and methods of this disclosure can include cells genetically engineered to be resistant to replicative senescence (RRS). In some embodiments, cells resistant to replicative senescence can comprise a transgene encoding B-cell lymphoma-extra large (Bcl-xL). In particular embodiments, cells resistant to replicative senescence can comprise a transgene encoding B-cell lymphoma-extra large (Bcl-xL) and/or B-cell lymphoma 2 (Bcl-2). In some embodiments, cells resistant to replicative senescence can comprise a knock-out of or have inhibited expression of one or more endogenous regulatory factors selected from cyclin-dependent kinase inhibitor 2A (CDKN2A), cyclin-dependent kinase inhibitor 2B (CDKN2B), and S-methyl-5′-thioadenosine phosphorylase (MTAP). In some embodiments, cells resistant to replicative senescence can comprise a knock-out of or inhibited expression of one or more endogenous immune related genes in the primary immune cells. In certain emobdiments, the endogenous immune related gene is beta-2 microglobulin (B2M), or T-cell receptor a constant (TRAC). In some embodiments, cells resistant to replicative senescence can comprise a knock-out of or inhibited expression of CD38.

The term “genetically engineered” refers to a change to the genetic material of the cell. A genetic edit includes genetic material to be added, removed, or altered in the genetically engineered cells. In particular embodiments, genetic edits comprise introducing a transgene into the cells and/or inhibiting the expression of a gene in the cell. In particular embodiments, introducing one or more genetic edits comprise introducing one or more transgenes encoding an anti-apoptotic factor or a virally-derived factor into the cells.

The term “transgene” refers to any nucleic acid sequence that is introduced into the cell by experimental manipulations. A transgene may be an “endogenous DNA sequence” or a “heterologous DNA sequence.” The term “endogenous” refers to developing or originating within a cell, a tissue, or an organism or part of a cell, a tissue or an organism. The transgene may be isolated and obtained in suitable quantity using one or more methods that are well known in the art. These methods and others useful for isolating a transgene are set forth, for example, in Sambrook et al. (supra) and in Berger and Kimmel (Methods in Enzymology: Guide to Molecular Cloning Techniques, vol. 152, Academic Press, Inc., San Diego, CA (1987)). The transgene can be incorporated into a “transgene construct” that comprises the gene of interest along with other regulatory DNA sequences needed either for temporal, or cell specific, or enhanced expression of the transgenes of interest. The transgene may be introduced into the cells by any suitable method or technique known in the art. In some embodiments, the transgene is introduced using a plasmid-based DNA transposon, lentivirus platform, or site-specific integration via CRISPR. The transgene expression in the cell can be constitutive or inducible.

In certain embodiments, the transgene encodes a virally-derived factor. A “virally-derived factor” refers to both naturally-occurring viral peptides, polypeptides, or proteins, as well as peptides, polypeptides, or proteins displaying a degree of sequence identity and/or similarity to a viral protein and/or maintaining one or more structural, mechanistic, or antigenic qualities of the viral protein. In particular embodiments, the virally-derived factor is from Saimiriine gammaherpesvirus 2 StpA All, Herpesvirus saimiri StpC, Herpesvirus saimiri Tip, or a modified Herpesvirus Ateles-Epstein-Barr virus Tio-LMP1.

In some embodiments, the cells as described herein further include inhibited expression of one or more endogenous regulatory factors in the cells such that the activity of the endogenous regulatory factor is eliminated or reduced. As used herein a “regulatory factor” refers to a gene that encodes a protein involved in regulating the cell cycle arrest, cell death, or signal suppression. The endogenous regulatory factor may be down regulated or blocked by any suitable method or technique known in the art. Known methods for down regulation of gene expression or decreasing the activity of a factor include, but are not limited to, CRISPR/Cas (including cytosine and adenine base editors), microRNA, shRNA, RNAi, TALENs, zinc finger nucleases, meganucleases, neutralizing antibodies, small molecule inhibitors, chemical inhibitors blocking downstream signaling pathways, and the like. The inhibition of the endogenous regulatory factor can be complete inhibition, partial inhibition, down regulation of gene expression or decreasing the activity of a factor. In some embodiments, endogenous regulatory factor activity or gene expression is reduced by between 1%-100% (i.e., 1%, 5%, 10%, 15%, 20%, 25%, 30%, 35%, 40%, 45%, 50%, 55%, 60%, 65%, 70%, 75%, 80%, 85%, 90%, 95%, 98%, 99%, 100%). A regulatory factor includes a gene that encodes a protein involved in regulating the cell cycle arrest, cell death or signal suppression. In particular aspects, the one or more endogenous regulatory factors are cyclin-dependent kinase inhibitor 2A (CDKN2A), cyclin-dependent kinase inhibitor 2B (CDKN2B), and/or S-methyl-5′-thioadenosine phosphorylase (MTAP). In particular embodiments, the one or more endogenous regulatory factors are RB Transcriptional Corepressor 1 (RB1), TP53, Autophagy and Beclin 1 Regulator 1 (AMBRA1), Neurofibromatosis type 1 (NF1), Tyrosine-protein phosphatase non-receptor type 2 (PTPN2), or Suppressor of Cytokine Signaling 1 (SOCS1).

In some embodiments, cells as disclosed herein include inhibited expression of one or more endogenous immune related genes in the cells such that the activity of the immune related genes is eliminated or reduced. As used herein an “immune related gene” refers to a gene that encodes a protein involved in effecting an immune response. In certain aspects, the immune related gene encodes a protein that is involved in host-versus-graft (HvG) and graft-versus-host (GvH) allogeneic immune responses. The immune related gene may be down regulated or blocked by any suitable method or technique known in the art. Known methods for down regulation of gene expression or decreasing the activity of an immune related gene include, but are not limited to, CRISPR/Cas (including cytosine and adenine base editors), microRNA, shRNA, RNAi, TALENs, zinc finger nucleases, meganucleases, neutralizing antibodies, small molecule inhibitors, chemical inhibitors blocking downstream signaling pathways, and the like. The inhibition of the endogenous immune related gene can be complete inhibition, partial inhibition, down regulation of gene expression or decreasing the activity of a factor. In some embodiments, endogenous immune related gene activity or gene expression is reduced by between 1%-100% (i.e., 1%, 5%, 10%, 15%, 20%, 25%, 30%, 35%, 40%, 45%, 50%, 55%, 60%, 65%, 70%, 75%, 80%, 85%, 90%, 95%, 98%, 99%, 100%). An immune related gene includes a gene that encodes a protein involved in effecting an immune response. An immune related gene can encode a protein that is involved in host-versus-graft (HvG) and graft-versus-host (GvH) allogeneic immune responses. In particular embodiments, the one or more endogenous immune related genes are beta-2 microglobulin (B2M), or T-cell receptor a constant (TRAC). In particular embodiments, the one or more endogenous immune related genes are genes of the major histocompatibility complex (MHC), human leukocyte antigen class I genes (e.g. HLA-A, HLA-B, HLA-C), human leukocyte antigen class II genes (HLA-DR, HLA-DQ, and HLA-DP), T cell receptors (e.g. aß T cell receptor), interleukin 1 (IL-1), interleukin 2 (IL-2), interleukin 4 (IL-4), interleukin 6 (IL-6), interleukin 10 (IL-10), interleukin 23 (IL-23), interferon-γ (IFNγ), CCL2, CCL3, CCL4, CCL5, CXCL2, CXCL9-11, CCL17, CCL27, programmed death-1 (PD-1), TIM3, or TIGIT.

In further embodiments, the cells as disclosed herein include inhibited expression of cluster of differentiation 38 (CD38) in the cells such that the activity of CD38 is eliminated or reduced. CD38 may be down regulated or blocked by any suitable method or technique known in the art. Known methods for down regulation of gene expression or decreasing the activity of CD38 include, but are not limited to, CRISPR/Cas (including cytosine and adenine base editors), microRNA, shRNA, RNAi, TALENs, zinc finger nucleases, meganucleases, neutralizing antibodies, small molecule inhibitors, chemical inhibitors blocking downstream signaling pathways, and the like. In some embodiments, CD38 activity or gene expression is reduced by between 1%-100% (i.e., 1%, 5%, 10%, 15%, 20%, 25%, 30%, 35%, 40%, 45%, 50%, 55%, 60%, 65%, 70%, 75%, 80%, 85%, 90%, 95%, 98%, 99%, 100%).

In further embodiments, the cells as disclosed herein include inhibited expression of phosphatase and tensin homolog (PTEN) in the primary immune cells such that the activity of PTEN is eliminated or reduced. PTEN may be down regulated or blocked by any suitable method or technique known in the art. Known methods for down regulation of gene expression or decreasing the activity of PTEN include, but are not limited to, CRISPR/Cas (including cytosine and adenine base editors), microRNA, shRNA, RNAi, TALENs, zinc finger nucleases, meganucleases, neutralizing antibodies, small molecule inhibitors, chemical inhibitors blocking downstream signaling pathways, and the like. In some embodiments, PTEN activity or gene expression is reduced by between 1%-100% (i.e., 1%, 5%, 10%, 15%, 20%, 25%, 30%, 35%, 40%, 45%, 50%, 55%, 60%, 65%, 70%, 75%, 80%, 85%, 90%, 95%, 98%, 99%, 100%).

The term “T_{REX}” refers to a “T cell that is Renewably Expandable” using e.g., the techniques and genetic modifications provided herein. More specifically, T_{REX} cells refer to cells with decreased or ablated expression of some or all of cyclin-dependent kinase inhibitor 2A (CDKN2A), cyclin-dependent kinase inhibitor 2B CDKN2B, and S-methyl-5′-thioadenosine phosphorylase (MTAP).

In some embodiments, inhibiting the expression of one or more endogenous regulatory factors occurs after introduction of the one or more transgene into the cells. In some aspects, cells in which one or more transgene has been introduced are cultured for at least 2 days, at least 5 days, at least 10 days, at least 11 days, at least 12 days, at least 13 days, at least 14 days, at least 15 days, at least 16 days, at least 17 days, at least 18 days, at least 19 days, at least 20 days before inhibition of one or more endogenous regulatory factor is performed. In further embodiments, inhibiting the expression of PTEN occurs after introduction of the one or more transgenes into the cells. In some embodiments, the method comprises the following sequential steps i) introducing one or more transgenes into the immune cells and then culturing the cells for at least 2 days, 5 days, at least 10 days, at least 11 days, at least 12 days, at least 13 days, at least 14 days, at least 15 days, at least 16 days, at least 17 days, at least 18 days, at least 19 days, at least 20 days; ii) inhibiting one or more endogenous regulatory factor culturing the cell for at least 2 days, 5 days, at least 10 days, at least 11 days, at least 12 days, at least 13 days, at least 14 days, at least 15 days, at least 16 days, at least 17 days, at least 18 days, at least 19 days, at least 20 days; and iii) inhibiting PTEN expression.

### SMART/Activation and Expansion of T Cells/T_{REX} Cells

The present disclosure also relates to culturing methods of T cells transduced with chimeric antigen receptors (CARs) that generate a persisting population of T cells that exhibit increased antigen-independent activation. The acronym “SMART” (Shorty-Manipulated Auto-Replicating T-Cells) refers to a shortened T cell manufacture and expansion process wherein the cells are cultured in the presence of IL-21 (and optionally IL-2).

Some aspects of the present disclosure are directed to cells comprising a polynucleotide or a polypeptide disclosed herein. Some aspects of the present disclosure are directed to a cell comprising (i) a polynucleotide encoding a chimeric antigen receptor (CAR) that binds human BCMA. In some embodiments, the cell further comprises (ii) a polynucleotide encoding an armoring molecule. In some embodiments, the cell is an immune cell. In some embodiments, the cell is an autologous cell to the recipient. In some embodiments, the cell is selected from the group consisting of a T cell, a Natural Killer (NK) cell, a cytotoxic T lymphocyte (CTL), a regulatory T cell, a γδ T cell, a TSCM cell, a CMV+ T cell, a tumor infiltrating lymphocyte, and any combination thereof. In some embodiments, the cell is a mammalian cell. In some embodiments, the cell is a human cell.

Prior to expansion and genetic modification of the T cells of the disclosure, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present disclosure, any number of T cell lines available in the art, may be used. In certain embodiments of the present disclosure, T cells can be obtained from a unit of blood collected from a subject using any number of techniques known to the skilled artisan, such as Ficoll™ separation. In one embodiment, cells from the circulating blood of an individual are obtained by apheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In some embodiments, the cells are washed with phosphate buffered saline (PBS). In an alternative embodiment, the wash solution lacks calcium and may lack magnesium or may lack many if not all divalent cations. Again, initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca2+-free, Mg2+-frec PBS, PlasmaLyte A, or other saline solution with or without buffer. Alternatively, the undesirable components of the apheresis sample may be removed and the cells directly resuspended in culture media.

In other embodiments, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3+, CD28+, CD4+, CD8+, CD45RA+, and CD45RO+ T cells, can be further isolated by positive or negative selection techniques. In some embodiments, T cells are isolated by positive selection for CD4 and CD8 expression. For example, in one embodiment, T cells are isolated by incubation with anti-CD4/anti-CD8-conjugated beads for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another embodiment, the time period is 10 to 24 hours. Longer incubation times may be used to isolate T cells in any situation where there are few T cells as compared to other cell types, such in isolating tumor infiltrating lymphocytes (TIL) from tumor tissue or from immune-compromised individuals. Further, use of longer incubation times can increase the efficiency of capture of CD8+ T cells. Thus, by simply shortening or lengthening the time T cells are allowed to bind to the CD4/CD8 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD4 and/or anti-CD8 antibodies on the beads or other surface, subpopulations of T cells can be preferentially selected for or against at culture initiation or at other desired time points. The skilled artisan would recognize that multiple rounds of selection can also be used in the context of this disclosure. In certain embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, and HLA-DR. In certain embodiments, it may be desirable to enrich for or positively select for regulatory T cells which typically express CD4+, CD25+, CD62Lhi, GITR+, and FoxP3+. Alternatively, in certain embodiments, T regulatory cells are depleted by anti-C25 conjugated beads or other similar method of selection.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one embodiment, the concentration of cells used is 5×106/ml. In other embodiments, the concentration used can be from about 1×10⁵/ml to 1×106/ml, and any integer value in between.

In other embodiments, the cells may be incubated on a rotator for varying lengths of time at varying speeds at either 2-10° C. or at room temperature.

T cells for stimulation can also be frozen after a washing step. In some embodiments, the freeze and subsequent thaw step can provide a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In certain embodiments, cryopreserved cells are thawed and washed and allowed to rest for one hour at room temperature prior to activation using the methods of the present disclosure.

Also contemplated in the context of this disclosure is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one embodiment a blood sample or an apheresis is taken from a generally healthy subject. In certain embodiments, a blood sample or an apheresis is taken from a generally healthy subject who is at risk of developing a disease, but who has not yet developed a disease, and the cells of interest are isolated and frozen for later use. In certain embodiments, the T cells may be expanded, frozen, and used at a later time. In certain embodiments, samples are collected from a patient shortly after diagnosis of a particular disease as described herein but prior to any treatments. In a further embodiment, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprinc, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation. These drugs inhibit either the calcium dependent phosphatase calcincurin (cyclosporine and FK506) or inhibit the p70S6 kinase that is important for growth factor induced signaling (rapamycin) (Liu et al., Cell 66:807-815, 1991; Henderson et al., Immun. 73:316-321, 1991; Bierer et al., Curr. Opin. Immun. 5:763-773, 1993). In a further embodiment, the cells are isolated for a patient and frozen for later use in conjunction with (e.g., before, simultaneously or following) bone marrow or stem cell transplantation, T cell ablative therapy using either chemotherapy agents such as, fludarabine, external-beam radiation therapy (XRT), cyclophosphamide, or antibodies such as OKT3 or CAMPATH. In another embodiment, the cells are isolated prior to and can be frozen for later use for treatment following B-cell ablative therapy such as agents that react with CD20, e.g., Rituxan.

In a further embodiment of the present disclosure, T cells are obtained from a patient directly following treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present disclosure to collect blood cells, including T cells, dendritic cells, or other cells of the hematopoietic lineage, during this recovery phase. Further, in certain embodiments, mobilization (for example, mobilization with GM-CSF or G-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

Whether prior to or after genetic modification of the T cells to express a desirable CAR, the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, the T cells of the disclosure are expanded by contact with a surface having attached thereto an agent that stimulates a CD3/TCR complex associated signal and a ligand that stimulates a costimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For costimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody. Examples of an anti-CD28 antibody include 9.3, B-T3, XR-CD28 (Diaclone, Besangon, France) can be used as can other methods commonly known in the art (Berg et al., Transplant Proc. 30 (8): 3975-3977, 1998; Haanen et al., J. Exp. Med. 190 (9): 13191328, 1999; Garland et al., J. Immunol Meth. 227 (1-2): 53-63, 1999).

In certain embodiments, the primary stimulatory signal and the costimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one embodiment, the agent providing the costimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing Fc receptors or an antibody or other binding agent which will bind to the agents. In this regard, sce for example, U.S. Patent Application Publication Nos. 20040101519 and 20060034810 for artificial antigen presenting cells (aAPCs) that are contemplated for use in activating and expanding T cells in the present disclosure.

In one embodiment, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the costimulatory signal is an anti-CD28 antibody or antigen-binding fragment thereof, and both agents are co-immobilized to the same bead in equivalent molecular amounts. In one embodiment, a 1:1 ratio of each antibody bound to the beads for CD4+ T cell expansion and T cell growth is used. In certain embodiments of the present disclosure, a ratio of anti CD3:CD28 antibodies bound to the beads is used such that an increase in T cell expansion is observed as compared to the expansion observed using a ratio of 1:1.

In further embodiments of the present disclosure, the cells, such as T cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), IL-21, insulin, IFN-7, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO2). In one embodiment, the media is X-VIVO 15 serum-free media containing 1% (v/v) recombinant serum replacement (ITSE-A).

In one embodiment, the T cells are cultured in media containing between 10 and 300 IU/mL of recombinant human IL-2. In one embodiment, the T cells are cultured in media containing 10, 15, 20, 25, 30, 35, 40, 45, 50, 55, 60, 65, 70, 75, 80, 85, 90, 95, 100, 200, or 300 IU/mL of recombinant human IL-2. In another embodiment, the T cells are cultured in media also containing between 0.1 and 0.3 U/mL of recombinant IL-21. In another embodiment, the T cells are cultured in media containing IL-2 and 0.1, 0.2, 0.5, 1, 2, 5, 10, 15, 20, 25, 30, 40, 50, 75, or 100 U/mL of recombinant human IL-21. In another embodiment, the T cells are culture in media containing IL-2 and 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, or 0.30 U/mL of recombinant human IL-21. In one embodiment, the T cells are cultured in a media containing 40 IU/mL of recombinant human IL-2 and 0.24 U/mL of recombinant human IL-21.

In one embodiment of the present disclosure, the cells cultured for up to 14 days. In another embodiment, the mixture may be cultured for 4 days. The T cells can be agitated during any stage of culture. In one embodiment, the cells are agitated during cell culture in media containing IL-2 and IL-21. In certain embodiments, the T cells harvested on day 4 exhibit higher target independent killing activity compared to CAR-T cells harvested on day 6.

In one embodiment of the present disclosure, CD8+ T cells are isolated from total PBMCs. Cells are either cryopreserved or activated immediately upon isolation using CD3/CD28 stimulation. Following 3 days of activation, CRISPR knockout of CDKN2A, CDKN2B, and MTAP (termed REX edits) is performed in order to confer resistance to replicative senescence. Cells are further manipulated with site specific CRISPR knock-in of a BCMA-CAR. In some embodiments, a knockout of B2M is performed to limit recognition of these donor cells by patient CD8⁺ T cells. In some embodiments, CD38 is additionally knocked out to enable resistance to Daratumumab. In some embodiments, cells are edited at the TRAC locus to ablate TCR αβ expression and eliminate the risk of graft-vs-host disease.

### Vectors, Host Cells, and Pharmaceutical Compositions of the Disclosure

In some embodiments, the polynucleotides of the present disclosure are present in a vector. As such, provided herein are vectors comprising the polynucleotides of the present disclosure. In some embodiments, the present disclosure is directed to a vector or a set of vectors comprising a polynucleotide encoding a CAR, as described herein.

Any vector known in the art can be suitable for the present disclosure. In some embodiments, the vector is a viral vector. In some embodiments, the vector is a retroviral vector, a DNA vector, a murine leukemia virus vector, an SFG vector, a plasmid, a RNA vector, an adenoviral vector, a baculoviral vector, an Epstein Barr viral vector, a papovaviral vector, a vaccinia viral vector, a herpes simplex viral vector, an adenovirus associated vector (AAV), a lentiviral vector, transposon, or any combination thereof. In certain embodiments, the CARs and/or antibodies or antigen binding fragments thereof are encompassed by and/or delivered to a cell and or patient using a virus, a lentivirus, an adenovirus, a retrovirus, an adeno-associated virus (AAV), a transposon, a DNA vector, a mRNA, a lipid nanoparticle (LNP), or a CRISPR-Cas System.

In other embodiments, provided herein are host cells comprising a polynucleotide or a vector of the present disclosure. In some embodiments, the present disclosure is directed to host cells, e.g., in vitro cells, comprising a polynucleotide encoding a CAR or a TCR, as described herein. In other embodiments, the present disclosure is directed to in vitro cells comprising a polypeptide encoded by a polynucleotide encoding a CAR that specifically binds to BCMA. In other embodiments, the present disclosure is directed to cells, e.g., in vitro cells, comprising a polypeptide encoded by a polynucleotide encoding an antibody or an antigen binding molecule thereof that specifically binds to BCMA, as disclosed herein.

Any cell can be used as a host cell for the polynucleotides, the vectors, or the polypeptides of the present disclosure. In some embodiments, the cell can be a prokaryotic cell, fungal cell, yeast cell, or higher eukaryotic cells such as a mammalian cell. Suitable prokaryotic cells include, without limitation, cubacteria, such as Gram-negative or Gram-positive organisms, for example, Enterobacteriaceae such as Escherichia, e.g., E. coli; Enterobacter; Erwinia; Klebsiella; Proteus; Salmonella, e.g., Salmonella typhimurium; Serratia, e.g., Serratia Marcescens, and Shigella; Bacilli such as B. subtilis and B. licheniformis; Pseudomonas such as P. aeruginosa; and Streptomyces. In some embodiments, the cell is a human cell.

Other embodiments of the present disclosure are directed to compositions comprising a polynucleotide described herein, a vector described herein, a polypeptide described herein, or cell described herein. In some embodiments, the composition comprises a pharmaceutically acceptable carrier, diluent, solubilizer, emulsifier, preservative and/or adjuvant. In some embodiments, the composition comprises an excipient. In one embodiment, the composition comprises a polynucleotide encoding a CAR, wherein the CAR comprises an antigen binding molecule that specifically binds to BCMA. In another embodiment, the composition comprises a CAR encoded by a polynucleotide of the present disclosure, wherein the CAR comprises an antigen binding molecule that specifically binds to BCMA. In another embodiment, the composition comprises a T cell comprising a polynucleotide encoding a CAR, wherein the CAR comprises an antigen binding molecule that specifically binds to BCMA. In another embodiment, the composition comprises a cell (e.g., a T cell, e.g., a CAR-T cell) comprising a polynucleotide encoding CAR comprising an antigen binding domain that specifically binds BCMA, as disclosed herein.

In other embodiments, the composition is formulated for parenteral delivery, for inhalation, or for delivery through the digestive tract, such as orally. The preparation of such pharmaceutically acceptable compositions is within the ability of one skilled in the art. In certain embodiments, buffers are used to maintain the composition at physiological pH or at a slightly lower pH, typically within a pH range of from about 5 to about 8. In certain embodiments, when parenteral administration is contemplated, the composition is in the form of a pyrogen-free, parenterally acceptable aqueous solution with or without additional therapeutic agents, in a pharmaceutically acceptable vehicle. In certain embodiments, the vehicle for parenteral injection is sterile distilled water with or without at least one additional therapeutic agent, is formulated as a sterile, isotonic solution, properly preserved. In certain embodiments, the preparation involves the formulation of the desired molecule with polymeric compounds (such as polylactic acid or polyglycolic acid), beads or liposomes, that provide for the controlled or sustained release of the product, which are then be delivered via a depot injection. In certain embodiments, implantable drug delivery devices are used to introduce the desired molecule.

Treatment of Disease with CARS

In some embodiments, the present disclosure provides CAR cells for treatment of disease. In certain embodiments, the present disclosure provides CAR cells for treatment of cancer and/or hematologic malignancies. In an embodiment, the present disclosure provides CAR cells for treatment of cancer and/or hematologic malignancies expressing BCMA. The compositions (e.g., CAR constructs, and CAR cells) and methods of their use described herein are especially useful for inhibiting neoplastic cell growth or spread; particularly neoplastic cell growth in which BCMA plays a role.

In one embodiment, cancers contemplated for treatment here include any that express BCMA on the cell surfaces of the cancer cells. Cancers contemplated for treatment herein can include, but are not limited to, multiple myeloma (MM), chronic lymphocytic leukemia, acute B-lymphoblastic leukemia, non-Hodgkin lymphoma (NHL), Hodgkin lymphoma, acute myeloid leukemia (AML), and acute lymphoblastic leukemia (ALL). In an embodiment, the present disclosure provides CAR cells for treatment of multiple myeloma.

In some embodiments, the present disclosure provides CAR cells for treatment of autoimmune disease. In certain embodiments, the present disclosure provides CAR cells for treatment of autoimmune diseases involving BCMA. The compositions (e.g., CAR constructs, and CAR cells) and methods of their use described herein are especially useful for treatment of autoimmune diseases in which BCMA plays a role. In certain embodiments, the present disclosure provides CAR cells for treatment of lupus.

### Methods of Treatment

CAR-modified cells of the present disclosure, such as CAR T cells, may be administered alone or as a pharmaceutical composition with a diluent and/or other components associated with cytokines or cell populations. Briefly, pharmaceutical compositions of the disclosure can include, for example CAR cells as described herein, with one or more pharmaceutically or physiologically acceptable carrier, diluent, or excipient. Such compositions can comprise buffers such as neutral buffered saline, buffered saline, and the like; sulfates; carbohydrates such as glucose, mannose, sucrose, or dextrans, mannitol; proteins, polypeptides, or amino acids such as glycine; antioxidants; chelating agents such as EDTA or glutathione; adjuvants (e.g., aluminum hydroxide); and preservatives. The pharmaceutical compositions of the disclosure may be adapted to the treatment (or prophylaxis).

In some embodiments, the present disclosure provides a method of treating a disease by administering to a subject in need thereof an effective amount of a cell comprising an anti-BCMA chimeric antigen receptor (CAR) comprising an antigen binding domain. The antigen binding domain can be an antibody, Fab, or an scFv comprising a heavy chain variable region (VH) and a light chain variable region (VL). In certain embodiments, the VH comprises a CDR1 comprising an amino acid sequence selected from SEQ ID NO: 2, 11, 20, 29, 38, 47, 56, 65, 74, and 83; a CDR2 comprising an amino acid sequence selected from SEQ ID NO: 3, 12, 21, 30, 39, 48, 57, 66, 75, and 84; and a CDR3 comprising an amino acid sequence selected from SEQ ID NO: 4, 13, 22, 31, 40, 49, 58, 67, 76, and 85; and the VL comprises a CDR1 comprising an amino acid sequence selected from SEQ ID NO: 6, 15, 24, 33, 42, 51, 60, 69, 78, and 87; a CDR2 comprising an amino acid sequence selected from SEQ ID NO: 7, 16, 25, 34, 43, 52, 61, 70, 79, and 88; and a CDR3 comprising an amino acid sequence selected from SEQ ID NO: 8, 17, 26, 35, 44, 53, 62, 71, 80, and 89. In certain embodiments, the VH comprises an amino acid sequence selected from SEQ ID NO: 1, 10, 19, 28, 37, 46, 55, 64, 73, and 82. In some embodiments, the VL comprises an amino acid sequence selected from SEQ ID NO: 5, 14, 23, 32, 41, 50, 59, 68, 77, and 86. In one aspect, this disclosure provides an anti-BCMA CAR comprising or consisting of the amino acid sequence set forth as SEQ ID NO: 96. In another aspect, this disclosure provides an anti-BCMA CAR comprising or consisting of the amino acid sequence set forth as SEQ ID NO: 97. In yet another aspect, this disclosure provides an anti-BCMA CAR comprising or consisting of the amino acid sequence set forth as SEQ ID NO: 98.

As used herein, the term an “effective amount” or a “therapeutically effective amount” of an administered therapeutic substance, such as a CAR T cell, is an amount sufficient to carry out a specifically stated or intended purpose, such as treating or treatment of a disease. An “effective amount” can be determined empirically in relation to the stated purpose. In certain embodiments, a therapeutically effective amount can refere to the number of cells administered to a subject in need of treatment. The number of cells per dose, the number of doses, and frequency of dosing will depend on various parameters such as the patient's age, weight, clinical assessment, disease type, cancer type, tumor type, tumor burden, and/or other factors, including the judgment of the attending physician.

In some embodiments, the cancer treated by the method is multiple myeloma (MM), chronic lymphocytic leukemia, acute B-lymphoblastic leukemia, non-Hodgkin lymphoma (NHL), Hodgkin lymphoma, acute myeloid leukemia (AML), or acute lymphoblastic leukemia (ALL). In an embodiment, the present disclosure provides CAR cells for treatment of multiple myeloma.

In some embodiments, the present disclosure provides CAR cells for treatment of autoimmune disease. In certain embodiments, the methods of present disclosure provide CAR cells for treatment of autoimmune diseases involving BCMA. The compositions (e.g., CAR constructs, and CAR cells) and methods of their use described herein are especially useful for treatment of autoimmunde diseases in which BCMA plays a role. In certain embodiments, the present disclosure provides CAR cells for treatment of lupus.

It is to be understood that the particular aspects of the specification are described herein are not limited to specific embodiments presented, and can vary. It also will be understood that the terminology used herein is for the purpose of describing particular aspects only and, unless specifically defined herein, is not intended to be limiting. Moreover, particular embodiments disclosed herein can be combined with other embodiments disclosed herein, as would be recognized by a skilled person, without limitation.

### EMBODIMENTS

Embodiment 1. An isolated nucleic acid sequence encoding a chimeric antigen receptor (CAR), wherein the CAR comprises:
(a) an antigen binding domain specific for B cell maturation antigen (BCMA);
(b) a transmembrane domain; and
(c) one or more intracellular domains.

Embodiment 2. The isolated nucleic acid sequence of embodiment 1, wherein the antigen binding domain comprises an antibody or antigen-binding fragment thereof, Fab, Fab′, F(ab′)2, Fd, Fv, single-chain fragment variable (scFv), single chain antibody, VHH, vNAR, nanobody (single-domain antibody), or any combination thereof.

Embodiment 3. The isolated nucleic acid sequence of embodiment 2, wherein the antigen binding domain is a single chain variable fragment (scFv).

Embodiment 4. The isolated nucleic acid sequence of embodiment 3, wherein the antigen binding domain is a scFv comprising an amino acid sequence selected from SEQ ID NO: 9, 36, and 90.

Embodiment 5. The isolated nucleic acid sequence of embodiment 3, wherein the antigen binding domain is a scFv comprising the amino acid sequence of SEQ ID NO: 9.

Embodiment 6. The isolated nucleic acid sequence of any one of embodiments 1 to 5, wherein the transmembrane domain comprises a transmembrane domain selected from the transmembrane domain of CD4, CD8α, or CD28.

Embodiment 7. The isolated nucleic acid sequence of embodiment 6, wherein the transmembrane domain comprises a CD28 transmembrane domain.

Embodiment 8. The isolated nucleic acid sequence of any one of embodiments 1 to 7, wherein the one or more intracellular domains comprises a costimulatory domain or a portion thereof.

Embodiment 9. The isolated nucleic acid sequence of embodiment 8, wherein the costimulatory domain comprises one or more of CD3z, 4-1BB, CD2, CD27, CD28, OX-40, ICOS, IL-2Rβ, GITR, MyD88/CD40a costimulatory domains, and/or variants thereof.

Embodiment 10. The isolated nucleic acid sequence of any one of embodiments 1 to 9, wherein the intracellular domain comprises a CD3z costimulatory domain and a CD28 costimulatory domain.

Embodiment 11. The isolated nucleic acid sequence of any one of embodiments 1 to 9, wherein the intracellular domain comprises a CD3z costimulatory domain and a 4-1BB costimulatory domain.

Embodiment 12. The isolated nucleic acid sequence of any one of embodiments 1 to 9, wherein the intracellular domain comprises a CD3z costimulatory domain, a CD28 costimulatory domain, and a 4-1BB costimulatory domain.

Embodiment 13. The isolated nucleic acid sequence of any one of embodiments 1 to 12, wherein the CAR further comprises a hinge/spacer domain, optionally, wherein the hinge/spacer domain is located between the antigen binding domain and the transmembrane domain.

Embodiment 14. The isolated nucleic acid sequence of embodiment 13, wherein the hinge/spacer domain comprises an IgG1 hinge domain or variants thereof, an IgG2 hinge domain or variants thereof, an IgG3 hinge domain or variants thereof, an IgG4 hinge domain or variants thereof, an IgG4P domain, a CD8 hinge domain or variants thereof or a CD28 hinge domain or variants thereof.

Embodiment 15. The isolated nucleic acid sequence of embodiment 14, wherein the hinge/spacer domain is an IgG4 hinge/spacer, or variants thereof, optionally an IgG4P hinge/spacer comprising an S241P mutation.

Embodiment 16. The isolated nucleic acid sequence of any one of embodiments 1 to 15, wherein the nucleic acid sequence encodes a CAR with an amino acid sequence as set forth in SEQ ID NO: 96.

Embodiment 17. The isolated nucleic acid sequence of any one of embodiments 1 to 15, wherein the nucleic acid sequence encodes a CAR with an amino acid sequence as set forth in SEQ ID NO: 97.

Embodiment 18. The isolated nucleic acid sequence of any one of embodiments 1 to 15, wherein the nucleic acid sequence encodes a CAR with an amino acid sequence as set forth in SEQ ID NO: 98.

Embodiment 19. An anti-BCMA chimeric antigen receptor (CAR) comprising an antigen binding domain, wherein the antigen binding domain comprises an antibody, Fab, or an scFv comprising a heavy chain variable region (VH) and a light chain variable region (VL);
wherein the VH comprises a CDR1 comprising an amino acid sequence selected from SEQ ID NO: 2, 29, and 83; a CDR2 comprising an amino acid sequence selected from SEQ ID NO: 3, 30, and 84; and a CDR3 comprising an amino acid sequence selected from SEQ ID NO: 4, 31, and 85; and
wherein the VL comprises a CDR1 comprising an amino acid sequence selected from SEQ ID NO: 6, 33, and 87; a CDR2 comprising an amino acid sequence selected from SEQ ID NO: 7, 34, and 88; and a CDR3 comprising an amino acid sequence selected from SEQ ID NO: 8, 35, and 89.

Embodiment 20. The anti-BCMA CAR of embodiment 19, wherein the VH comprises an amino acid sequence selected from SEQ ID NO: 1, 28, and 82.

Embodiment 21. The anti-BCMA CAR of either embodiment 19 or 28, wherein the VL comprises an amino acid sequence selected from SEQ ID NO: 5, 32, and 86.

Embodiment 22. An anti-BCMA chimeric antigen receptor (CAR) comprising an antigen binding domain, wherein the antigen binding domain comprises an antibody, Fab, or an scFv comprising a heavy chain variable region (VH) and a light chain variable region (VL);
wherein the VH comprises a CDR1 comprising the amino acid sequence of SEQ ID NO: 2; a CDR2 comprising the amino acid sequence of SEQ ID NO: 3; and a CDR3 comprising the amino acid sequence of SEQ ID NO: 4; and
wherein the VL comprises a CDR1 comprising the amino acid sequence of SEQ ID NO: 6; a CDR2 comprising the amino acid sequence of SEQ ID NO: 7; and a CDR3 comprising the amino acid sequence of SEQ ID NO: 8.

Embodiment 23. The anti-BCMA CAR of any one of embodiments 19 to 22, wherein the VH comprises the amino acid sequence of SEQ ID NO: 1, and the VL comprises the amino acid sequence of SEQ ID NO: 5.

Embodiment 24. The anti-BCMA CAR of embodiment 19 to 23, wherein the CAR comprises a transmembrane domain, and one or more intracellular domains.

Embodiment 25. The anti-BCMA CAR of any one of embodiments 19 to 24, wherein the transmembrane domain comprises a transmembrane domain selected from the transmembrane domain of CD4, CD8α, or CD28.

Embodiment 26. The anti-BCMA CAR of embodiment 25, wherein the transmembrane domain comprises a CD28 transmembrane domain.

Embodiment 27. The anti-BCMA CAR of any one of embodiments 19 to 26, wherein the one or more intracellular domains comprises a costimulatory domain or a portion thereof.

Embodiment 28. The anti-BCMA CAR of embodiment 27, wherein the costimulatory domain comprises one or more of CD32, 4-1BB, CD2, CD27, CD28, OX-40, ICOS, IL-2Rβ, GITR, MyD88/CD40a costimulatory domains, and/or variants thereof.

Embodiment 29. The anti-BCMA CAR of any one of embodiments 24 to 28, wherein the intracellular domain comprises a CD3z costimulatory domain and a CD28 costimulatory domain.

Embodiment 30. The anti-BCMA CAR of any one of embodiments 24 to 28, wherein the intracellular domain comprises a CD3z costimulatory domain and a 4-1BB costimulatory domain.

Embodiment 31. The anti-BCMA CAR of any one of embodiments 24 to 28, wherein the intracellular domain comprises a CD3z costimulatory domain, a CD28 costimulatory domain, and a 4-1BB costimulatory domain.

Embodiment 32. The anti-BCMA CAR of any one of embodiments 19 to 31, wherein the CAR further comprises a hinge/spacer domain, optionally, wherein the hinge/spacer domain is located between the antigen binding domain and the transmembrane domain.

Embodiment 33. The anti-BCMA CAR of embodiment 32, wherein the hinge/spacer domain comprises an IgG1 hinge domain or variants thereof, an IgG2 hinge domain or variants thereof, an IgG3 hinge domain or variants thereof, an IgG4 hinge domain or variants thereof, an IgG4P domain, a CD8a hinge domain or variants thereof, or a CD28 hinge domain or variants thereof.

Embodiment 34. The anti-BCMA CAR of embodiment 33, wherein the hinge/spacer domain is an IgG4 hinge/spacer, or variants thereof, optionally an IgG4P hinge/spacer comprising an S241P mutation.

Embodiment 35. The anti-BCMA CAR of any one of embodiments 19 to 34, wherein the CAR has an amino acid sequence as set forth in SEQ ID NO: 96.

Embodiment 36. The anti-BCMA CAR of any one of embodiments 19 to 34, wherein the CAR has an amino acid sequence as set forth in SEQ ID NO: 97.

Embodiment 37. The anti-BCMA CAR of any one of embodiments 19 to 34, wherein the CAR has an amino acid sequence as set forth in SEQ ID NO: 98.

Embodiment 38. A vector comprising the isolated nucleic acid sequence of any one of embodiments 1-18 or encoding the chimeric antigen receptor of any one of embodiments 19-37, optionally wherein the vector is a virus, a lentivirus, an adenovirus, a retrovirus, an adeno-associated virus (AAV), a transposon, a DNA vector, a mRNA, a lipid nanoparticle (LNP), or a CRISPR-Cas System.

Embodiment 39. A cell comprising the vector of embodiment 38.

Embodiment 40. A cell comprising a nucleic acid sequence encoding the chimeric antigen receptor (CAR) of any one of embodiments 19-37 further comprising decreased expression or knock out of one or more endogenous regulatory factors.

Embodiment 41. The cell of embodiment 40, wherein the one or more endogenous regulatory factors are selected from cyclin-dependent kinase inhibitor 2A (CDKN2A), cyclin-dependent kinase inhibitor 2B (CDKN2B), and S-methyl-5′-thioadenosine phosphorylase (MTAP).

Embodiment 42. The cell of any one of embodiments 39 to 41, wherein the cell has decreased expression or knock out of CDKN2A, CDKN2B, and MTAP.

Embodiment 43. The cell of any one of embodiments 39 to 42, wherein the cell does not express phosphatase and tensin homolog (PTEN).

Embodiment 44. The cell of any one of embodiments 39 to 43 further comprising a transgene encoding either B-cell lymphoma-extra large (Bcl-xL) or B-cell lymphoma 2 (Bcl-2).

Embodiment 45. The cell of any one of embodiments 39 to 44, wherein the cell does not express of one or more endogenous immune related genes.

Embodiment 46. The cell of embodiment 45, wherein the endogenous immune related gene is beta-2 microglobulin (B2M), and/or T-cell receptor a constant (TRAC).

Embodiment 47. The cell of any one of embodiments 39 to 46, wherein the cell does not express cluster of differentiation 38 (CD38).

Embodiment 48. A cell comprising a BCMA specific antigen binding domain, wherein the antigen binding domain comprises an antibody, Fab, or an scFv comprising a heavy chain variable region (VH) and a light chain variable region (VL);

wherein the VH comprises a CDR1 comprising an amino acid sequence selected from SEQ ID NO: 2, 29, and 83; a CDR2 comprising an amino acid sequence selected from SEQ ID NO: 3, 30, and 84; and a CDR3 comprising an amino acid sequence selected from SEQ ID NO: 4, 31, and 85; and wherein the VL comprises a CDR1 comprising an amino acid sequence selected from SEQ ID NO: 6, 33, and 87; a CDR2 comprising an amino acid sequence selected from SEQ ID NO: 7, 34, and 88; and a CDR3 comprising an amino acid sequence selected from SEQ ID NO: 8, 35, and 89.

Embodiment 49. The cell of embodiment 48, wherein the VH comprises an amino acid sequence selected from SEQ ID NO: 1, 28, and 82.

Embodiment 50. The cell of either embodiment 48 or 49, wherein the VL comprises an amino acid sequence selected from SEQ ID NO: 5, 32, and 86.

Embodiment 51. A cell comprising a BCMA specific antigen binding domain, wherein the antigen binding domain comprises an antibody, Fab, or an scFv comprising a heavy chain variable region (VH) and a light chain variable region (VL);
wherein the VH comprises a CDR1 comprising the amino acid sequence of SEQ ID NO: 2; a CDR2 comprising the amino acid sequence of SEQ ID NO: 3; and a CDR3 comprising the amino acid sequence of SEQ ID NO: 4; and
wherein the VL comprises a CDR1 comprising the amino acid sequence of SEQ ID NO: 6; a CDR2 comprising the amino acid sequence of SEQ ID NO: 7; and a CDR3 comprising the amino acid sequence of SEQ ID NO: 8.

Embodiment 52. The cell of any one of embodiments 48 to 51, wherein the VH comprises the amino acid sequence of SEQ ID NO: 1, and the VL comprises the amino acid sequence of SEQ ID NO: 5.

Embodiment 53. The cell of any one of embodiments 48 to 52 further comprising decreased expression or knock out of one or more endogenous regulatory factors.

Embodiment 54. The cell of embodiment 53, wherein the one or more endogenous regulatory factors are selected from cyclin-dependent kinase inhibitor 2A (CDKN2A), cyclin-dependent kinase inhibitor 2B (CDKN2B), and S-methyl-5′-thioadenosine phosphorylase (MTAP).

Embodiment 55. The cell of any one of embodiments 48 to 54, wherein the cell has decreased expression or knock out of CDKN2A, CDKN2B, and MTAP.

Embodiment 56. The cell of any one of embodiments 48 to 55, wherein the cell does not express phosphatase and tensin homolog (PTEN).

Embodiment 57. The cell of any one of embodiments 48 to 56, wherein the cell further comprises a transgene encoding either B-cell lymphoma-extra large (Bcl-xL) or B-cell lymphoma 2 (Bcl-2).

Embodiment 58. The cell of any one of embodiments 48 to 57, wherein the cell does not express of one or more endogenous immune related genes.

Embodiment 59. The cell of embodiment 58, wherein the endogenous immune related gene is beta-2 microglobulin (B2M), and/or T-cell receptor a constant (TRAC).

Embodiment 60. The cell of any one of embodiments 48 to 59, wherein the cell does not express cluster of differentiation 38 (CD38).

Embodiment 61. A cell comprising a BCMA specific antigen binding domain, wherein the antigen binding domain comprises an antibody, Fab, or an scFv comprising a heavy chain variable region (VH) and a light chain variable region (VL);
wherein the VH comprises a CDR1 comprising the amino acid sequence of SEQ ID NO: 2; a CDR2 comprising the amino acid sequence of SEQ ID NO: 3; and a CDR3 comprising the amino acid sequence of SEQ ID NO: 4; and
wherein the VL comprises a CDR1 comprising the amino acid sequence of SEQ ID NO: 6; a CDR2 comprising the amino acid sequence of SEQ ID NO: 7; and a CDR3 comprising the amino acid sequence of SEQ ID NO: 8; and
wherein the cell comprises decreased expression or knock out of CDKN2A, CDKN2B, MTAP, B2M, TRAC, and CD38.

Embodiment 62. The cell of embodiment 61, wherein the VH comprises the amino acid sequence of SEQ ID NO: 1, and the VL comprises the amino acid sequence of SEQ ID NO: 5.

Embodiment 63. The cell of either embodiment 61 or embodiment 62, wherein the BCMA specific antigen binding domain comprises an amino acid sequence as set forth in SEQ ID NO: 96.

Embodiment 64. The cell of any one of embodiments 48 to 63, wherein the cell is selected from a T cell, a Natural Killer (NK) cell, a cytotoxic T lymphocyte (CTL), a tumor infiltrating lymphocyte, a regulatory T cell, a CD8+ T cell, a CD4+ T cell, a gamma-delta T cell, a mucosal associated invariant T (MAIT) T cell, a natural killer T (NKT) cell, and/or a combination thereof.

Embodiment 65. A method of treating a disease, comprising:
administering to a subject in need thereof an effective amount of a cell comprising an anti-BCMA chimeric antigen receptor (CAR) comprising an antigen binding domain, wherein the antigen binding domain comprises an antibody, Fab, or an scFv comprising a heavy chain variable region (VH) and a light chain variable region (VL),
wherein the VH comprises a CDR1 comprising an amino acid sequence selected from SEQ ID NO: 2, 29, and 83; a CDR2 comprising an amino acid sequence selected from SEQ ID NO: 3, 30, and 84; and a CDR3 comprising an amino acid sequence selected from SEQ ID NO: 4, 31, and 85; and
wherein the VL comprises a CDR1 comprising an amino acid sequence selected from SEQ ID NO: 6, 33, and 87; a CDR2 comprising an amino acid sequence selected from SEQ ID NO: 7, 34, and 88; and a CDR3 comprising an amino acid sequence selected from SEQ ID NO: 8, 35, and 89.

Embodiment 66. The method of embodiment 65, wherein the VH comprises an amino acid sequence selected from SEQ ID NO: 1, 28, and 82.

Embodiment 67. The method of either embodiment 65 or embodiment 66, wherein the VL comprises an amino acid sequence selected from SEQ ID NO: 5, 32, and 86.

Embodiment 68. A method of treating a disease, comprising:
administering to a subject in need thereof an effective amount of a cell comprising an anti-BCMA chimeric antigen receptor (CAR) comprising an antigen binding domain, wherein the antigen binding domain comprises an antibody, Fab, or an scFv comprising a heavy chain variable region (VH) and a light chain variable region (VL),
wherein the VH comprises a CDR1 comprising the amino acid sequence of SEQ ID NO: 2; a CDR2 comprising the amino acid sequence of SEQ ID NO: 3; and a CDR3 comprising the amino acid sequence of SEQ ID NO: 4; and
wherein the VL comprises a CDR1 comprising the amino acid sequence of SEQ ID NO: 6; a CDR2 comprising the amino acid sequence of SEQ ID NO: 7; and a CDR3 comprising the amino acid sequence of SEQ ID NO: 8.

Embodiment 69. The method of any one of embodiments 65 to 68, wherein the VH comprises the amino acid sequence of SEQ ID NO: 1, and the VL comprises the amino acid sequence of SEQ ID NO: 5.

Embodiment 70. The method of any one of embodiments 65 to 69 further comprising inhibiting cancer growth, inducing cancer regression, and/or prolonging survival of the subject.

Embodiment 71. The method of any one of embodiments 65 to 70, wherein the cell further comprises decreased expression or knock out of one or more endogenous regulatory factors.

Embodiment 72. The method of embodiment 71, wherein the one or more endogenous regulatory factors are selected from cyclin-dependent kinase inhibitor 2A (CDKN2A), cyclin-dependent kinase inhibitor 2B (CDKN2B), and S-methyl-5′-thioadenosine phosphorylase (MTAP).

Embodiment 73. The method of any one of embodiments 65 to 72, wherein the cell has decreased expression or knock out of CDKN2A, CDKN2B, and MTAP.

Embodiment 74. The method of any one of embodiments 65 to 73, wherein the cell does not express phosphatase and tensin homolog (PTEN).

Embodiment 75. The method of any one of embodiments 65 to 74, wherein the cell further comprises a transgene encoding either B-cell lymphoma-extra large (Bcl-xL) or B-cell lymphoma 2 (Bcl-2).

Embodiment 76. The method of any one of embodiments 65 to 75, wherein the cell does not express of one or more endogenous immune related genes.

Embodiment 77. The method of embodiment 76, wherein the endogenous immune related gene is beta-2 microglobulin (B2M), and/or T-cell receptor a constant (TRAC).

Embodiment 78. The cell of any one of embodiments 65 to 77, wherein the cell does not express cluster of differentiation 38 (CD38).

Embodiment 79. The method of any one of embodiments 65 to 78, wherein the cell is an autologous cell.

Embodiment 80. The method of any one of embodiments 65 to 78, wherein the cell is an allogeneic cell.

Embodiment 81. The method of any one of embodiments 65 to 80, wherein the cell is selected from a T cell, a Natural Killer (NK) cell, a cytotoxic T lymphocyte (CTL), a tumor infiltrating lymphocyte, a regulatory T cell, a CD8+ T cell, a CD4+ T cell, a gamma-delta T cell, a mucosal associated invariant T (MAIT) T cell, a natural killer T (NKT) cell, and/or a combination thereof.

Embodiment 82. The method of any one of embodiments 65 to 81, wherein the disease is a cancer selected from multiple myeloma (MM), chronic lymphocytic leukemia, acute B-lymphoblastic leukemia, non-Hodgkin lymphoma (NHL), Hodgkin lymphoma, acute myeloid leukemia (AML), and acute lymphoblastic leukemia (ALL).

Embodiment 83. The method of embodiment 82, wherein the cancer is multiple myeloma.

Embodiment 84. The method of any one of embodiments 65 to 81, wherein the disease is an autoimmune disease.

Embodiment 85. The method of embodiment 84, wherein the autoimmune disease is lupus.

Embodiment 86. A pharmaceutical composition comprising the isolated nucleic acid of any one of embodiments 1 to 18, the anti-BCMA CAR of any one of embodiments 19 to 37, the vector of embodiment 38, or the cell of any one of embodiments 39 to 64, and a pharmaceutically acceptable excipient.

Embodiment 87. A method of treating a disease in a subject in need thereof, comprising administering to the subject the isolated nucleic acid of any one of embodiments 1 to 18, the anti-BCMA CAR of any one of embodiments 19 to 37, the vector of embodiment 38, the cell of any one of embodiments 39 to 64, or the pharmaceutical composition of embodiment 78.

Embodiment 88. The method of embodiment 87, wherein the disease is a cancer or an autoimmune disease.

Embodiment 89. The method of embodiment 88, wherein the cancer is selected from multiple myeloma (MM), chronic lymphocytic leukemia, acute B-lymphoblastic leukemia, non-Hodgkin lymphoma (NHL), Hodgkin lymphoma, acute myeloid leukemia (AML), and acute lymphoblastic leukemia (ALL).

Embodiment 90. The method of embodiment 89, wherein the cancer is multiple myeloma.

Embodiment 91. The method of embodiment 88, wherein the autoimmune disease is lupus.

Embodiment 92. Use of the isolated nucleic acid of any one of embodiments 1 to 18, the anti-BCMA CAR of any one of embodiments 19 to 37, the vector of embodiment 38, the cell of any one of embodiments 39 to 64, or the pharmaceutical composition of embodiment 78 in the treatment of a disease in a subject in need thereof.

Embodiment 93. The use of embodiment 92, wherein the disease is a cancer or an autoimmune disease.

Embodiment 94. The use of embodiment 93, wherein the cancer is selected from multiple myeloma (MM), chronic lymphocytic leukemia, acute B-lymphoblastic leukemia, non-Hodgkin lymphoma (NHL), Hodgkin lymphoma, acute myeloid leukemia (AML), and acute lymphoblastic leukemia (ALL).

Embodiment 95. The use of embodiment 94, wherein the cancer is multiple myeloma.

Embodiment 96. The use of embodiment 93, wherein the autoimmune disease is lupus.

Embodiment 97. Use of an engineered cell for the manufacture of a medicament for treating a disease in a patient, wherein the engineered cell comprises an anti-BCMA chimeric antigen receptor (CAR) comprising an antigen binding domain, wherein the antigen binding domain comprises an antibody, Fab, or an scFv comprising a heavy chain variable region (VH) and a light chain variable region (VL),
wherein the VH comprises a CDR1 comprising an amino acid sequence selected from SEQ ID NO: 2, 29, and 83; a CDR2 comprising an amino acid sequence selected from SEQ ID NO: 3, 30, and 84; and a CDR3 comprising an amino acid sequence selected from SEQ ID NO: 4, 31, and 85; and
wherein the VL comprises a CDR1 comprising an amino acid sequence selected from SEQ ID NO: 6, 33, and 87; a CDR2 comprising an amino acid sequence selected from SEQ ID NO: 7, 34, and 88; and a CDR3 comprising an amino acid sequence selected from SEQ ID NO: 8, 35, and 89.

Embodiment 98. The use of embodiment 99, wherein the VH comprises a CDR1 comprising the amino acid sequence of SEQ ID NO: 2; a CDR2 comprising the amino acid sequence of SEQ ID NO: 3; and a CDR3 comprising the amino acid sequence of SEQ ID NO: 4; and wherein the VL comprises a CDR1 comprising the amino acid sequence of SEQ ID NO: 6; a CDR2 comprising the amino acid sequence of SEQ ID NO: 7; and a CDR3 comprising the amino acid sequence of SEQ ID NO: 8.

Embodiment 99. The use of embodiment 97, wherein the VH comprises an amino acid sequence selected from SEQ ID NO: 1, 28, and 82.

Embodiment 100. The use of embodiment 97, wherein the VL comprises an amino acid sequence selected from SEQ ID NO: 5, 32, and 86.

Embodiment 101. The use of any one of embodiments 97 to 100, wherein the VH comprises the amino acid sequence of SEQ ID NO: 1, and the VL comprises the amino acid sequence of SEQ ID NO: 5.

Embodiment 102. The use of any one of embodiments 97 to 101, wherein the CAR has an amino acid sequence as set forth in SEQ ID NO: 96.

Embodiment 103. The use of any one of embodiments 97 to 100, wherein the CAR has an amino acid sequence as set forth in SEQ ID NO: 97.

Embodiment 104. The use of any one of embodiments 97 to 100, wherein the CAR has an amino acid sequence as set forth in SEQ ID NO: 98.

Embodiment 105. The use of any one of embodiments 97 to 104 further comprising inhibiting cancer growth, inducing cancer regression, and/or prolonging survival of the subject.

Embodiment 106. The use of any one of embodiments 97 to 105, wherein the engineered cell further comprises decreased expression or knock out of one or more endogenous regulatory factors.

Embodiment 107. The use of embodiment 106, wherein the one or more endogenous regulatory factors are selected from cyclin-dependent kinase inhibitor 2A (CDKN2A), cyclin-dependent kinase inhibitor 2B (CDKN2B), and S-methyl-5′-thioadenosine phosphorylase (MTAP).

Embodiment 108. The use of any one of embodiments 97 to 107, wherein the engineered cell has decreased expression or knock out of CDKN2A, CDKN2B, and MTAP.

Embodiment 109. The use of any one of embodiments 97 to 108, wherein the engineered cell does not express phosphatase and tensin homolog (PTEN).

Embodiment 110. The use of any one of embodiments 97 to 109, wherein the engineered cell further comprises a transgene encoding either B-cell lymphoma-extra large (Bcl-xL) or B-cell lymphoma 2 (Bcl-2).

Embodiment 111. The use of any one of embodiments 97 to 110, wherein the engineered cell does not express of one or more endogenous immune related genes.

Embodiment 112. The use of embodiment 111, wherein the endogenous immune related gene is beta-2 microglobulin (B2M), and/or T-cell receptor a constant (TRAC).

Embodiment 113. The use of any one of embodiments 97 to 112, wherein the engineered cell does not express cluster of differentiation 38 (CD38).

Embodiment 114. The use of any one of embodiments 97 to 113, wherein the engineered cell is an autologous cell.

Embodiment 115. The use of any one of embodiments 97 to 113, wherein the engineered cell is an allogeneic cell.

Embodiment 116. The use of any one of embodiments 97 to 115, wherein the engineered cell is selected from a T cell, a Natural Killer (NK) cell, a cytotoxic T lymphocyte (CTL), a tumor infiltrating lymphocyte, a regulatory T cell, a CD8+ T cell, a CD4+ T cell, a gamma-delta T cell, a mucosal associated invariant T (MAIT) T cell, a natural killer T (NKT) cell, and/or a combination thereof.

Embodiment 117. The use of any one of embodiments 97 to 116, wherein the disease is a cancer selected from multiple myeloma (MM), chronic lymphocytic leukemia, acute B-lymphoblastic leukemia, non-Hodgkin lymphoma (NHL), Hodgkin lymphoma, acute myeloid leukemia (AML), and acute lymphoblastic leukemia (ALL).

Embodiment 118. The use of embodiment 117, wherein the cancer is multiple myeloma.

Embodiment 119. The use of any one of embodiments 97 to 116, wherein the disease is an autoimmune disease.

Embodiment 120. The use of embodiment 119, wherein the autoimmune disease is lupus.

Embodiment 121. An engineered cell for the manufacture of a medicament for treating a disease in a patient, wherein the engineered cell comprises an anti-BCMA chimeric antigen receptor (CAR) comprising an antigen binding domain, wherein the antigen binding domain comprises an antibody, Fab, or an scFv comprising a heavy chain variable region (VH) and a light chain variable region (VL),
wherein the VH comprises a CDR1 comprising an amino acid sequence selected from SEQ ID NO: 2, 29, and 83; a CDR2 comprising an amino acid sequence selected from SEQ ID NO: 3, 30, and 84; and a CDR3 comprising an amino acid sequence selected from SEQ ID NO: 4, 31, and 85; and
wherein the VL comprises a CDR1 comprising an amino acid sequence selected from SEQ ID NO: 6, 33, and 87; a CDR2 comprising an amino acid sequence selected from SEQ ID NO: 7, 34, and 88; and a CDR3 comprising an amino acid sequence selected from SEQ ID NO: 8, 35, and 89

Embodiment 122. The engineered cell of embodiment 121, wherein the VH comprises a CDR1 comprising the amino acid sequence of SEQ ID NO: 2; a CDR2 comprising the amino acid sequence of SEQ ID NO: 3; and a CDR3 comprising the amino acid sequence of SEQ ID NO: 4; and wherein the VL comprises a CDR1 comprising the amino acid sequence of SEQ ID NO: 6; a CDR2 comprising the amino acid sequence of SEQ ID NO: 7; and a CDR3 comprising the amino acid sequence of SEQ ID NO: 8.

Embodiment 123. The engineered cell of embodiment 121, wherein the VH comprises an amino acid sequence selected from SEQ ID NO: 1, 28, and 82.

Embodiment 124. The engineered cell of embodiment 121, wherein the VL comprises an amino acid sequence selected from SEQ ID NO: 5, 32, and 86.

Embodiment 125. The engineered cell of any one of embodiments 121 to 124, wherein the VH comprises the amino acid sequence of SEQ ID NO: 1, and the VL comprises the amino acid sequence of SEQ ID NO: 5.

Embodiment 126. The engineered cell of any one of embodiments 121 to 125, wherein the CAR has an amino acid sequence as set forth in SEQ ID NO: 96.

Embodiment 127. The engineered cell of any one of embodiments 121 to 124, wherein the CAR has an amino acid sequence as set forth in SEQ ID NO: 97.

Embodiment 128. The engineered cell of any one of embodiments 121 to 124, wherein the CAR has an amino acid sequence as set forth in SEQ ID NO: 98.

Embodiment 129. The engineered cell of any one of embodiments 121 to 128 further comprising inhibiting cancer growth, inducing cancer regression, and/or prolonging survival of the subject.

Embodiment 130. The engineered cell of any one of embodiments 121 to 129, wherein the engineered cell further comprises decreased expression or knock out of one or more endogenous regulatory factors.

Embodiment 131. The engineered cell of embodiment 130, wherein the one or more endogenous regulatory factors are selected from cyclin-dependent kinase inhibitor 2A (CDKN2A), cyclin-dependent kinase inhibitor 2B (CDKN2B), and S-methyl-5′-thioadenosine phosphorylase (MTAP).

Embodiment 132. The engineered cell of any one of embodiments 121 to 131, wherein the engineered cell has decreased expression or knock out of CDKN2A, CDKN2B, and MTAP.

Embodiment 133. The engineered cell of any one of embodiments 121 to 132, wherein the engineered cell does not express phosphatase and tensin homolog (PTEN).

Embodiment 134. The engineered cell of any one of embodiments 121 to 133, wherein the engineered cell further comprises a transgene encoding either B-cell lymphoma-extra large (Bcl-xL) or B-cell lymphoma 2 (Bcl-2).

Embodiment 135. The engineered cell of any one of embodiments 121 to 134, wherein the engineered cell does not express of one or more endogenous immune related genes.

Embodiment 136. The engineered cell of embodiment 135, wherein the endogenous immune related gene is beta-2 microglobulin (B2M), and/or T-cell receptor a constant (TRAC).

Embodiment 137. The engineered cell of any one of embodiments 121 to 136, wherein the engineered cell does not express cluster of differentiation 38 (CD38).

Embodiment 138. The engineered cell of any one of embodiments 121 to 137, wherein the engineered cell is an autologous cell.

Embodiment 139. The engineered cell of any one of embodiments 121 to 138, wherein the engineered cell is an allogeneic cell.

Embodiment 140. The engineered cell of any one of embodiments 121 to 139, wherein the engineered cell is selected from a T cell, a Natural Killer (NK) cell, a cytotoxic T lymphocyte (CTL), a tumor infiltrating lymphocyte, a regulatory T cell, a CD8+ T cell, a CD4+ T cell, a gamma-delta T cell, a mucosal associated invariant T (MAIT) T cell, a natural killer T (NKT) cell, and/or a combination thereof.

Embodiment 141. The engineered cell of any one of embodiments 121 to 140, wherein the disease is a cancer selected from multiple myeloma (MM), chronic lymphocytic leukemia, acute B-lymphoblastic leukemia, non-Hodgkin lymphoma (NHL), Hodgkin lymphoma, acute myeloid leukemia (AML), and acute lymphoblastic leukemia (ALL).

Embodiment 142. The engineered cell of embodiment 141, wherein the cancer is multiple myeloma.

Embodiment 143. The engineered cell of any one of embodiments 121 to 142, wherein the disease is an autoimmune disease.

Embodiment 144. The engineered cell of embodiment 143, wherein the autoimmune disease is lupus.

### EXAMPLES

The Examples that follow are illustrative of specific embodiments of the disclosure, and various uses thereof. They are set forth for explanatory purposes only and should not be construed as limiting the scope of the disclosure in any way.

### Materials and Methods

### Cell Lines:

All cells were cultured in medium as per supplier recommendations and maintained in tissue culture flasks at 37° C. in a humidified atmosphere at 5% CO2. JJN3, U226B1, MM1S, KMS12BM, KMS34, KMS11, RPMI 8226, NCIH929, Huh7 cell lines were obtained from the American Tissue Culture Collection (ATCC, Manassas, VA) (DSMZ, Braunschweig, Germany).

### Lentivirus Prep

Lentiviral vectors were prepared by co-transfecting suspension adapted HEK293 cells with a proprietary lentiviral transfer vector and a commercially available packaging plasmid mix (pPACKHI, System Biosciences, Palo Alto, CA, USA). Transfected cells were cultured for 24 hours, then transferred into fresh media. At 48 hours post-transfection, cells were cleared by centrifugation and lentivirus containing supernatant was recovered.

Lentiviral particles were purified and concentrated by precipitation using the PEG-it precipitation reagent (System Biosciences) according to manufacturer provided protocols. After precipitation from culture supernatant, viral particles were collected by centrifugation and resuspended in 1/100 original volume of media. Functional titers were determined by transducing HEK-293 cells with serial dilutions of purified, concentrated vírus. On day 5 post-transduction, cells were labeled with fluorophore-conjugated rBCMA to determine the percentage, and by extension, number of cells transduced with the anti-BCMA CAR constructs. Titer values (in tfu/mL) were calculated by linear fit of number of cells transduced vs. Volume of virus added.

### Example 1. Development and Characterization of Anti-BCMA Antigen Binding Fragments

BCMA-reactive human antibodies were generated through immunization of transgenic mice (human variable domain repertoires) and hybridoma generation. Antibodies produced by individual hybridoma clones were assayed for binding to human BCMA in ELISA assays and antibody V region sequences were recovered for binding competent clones.

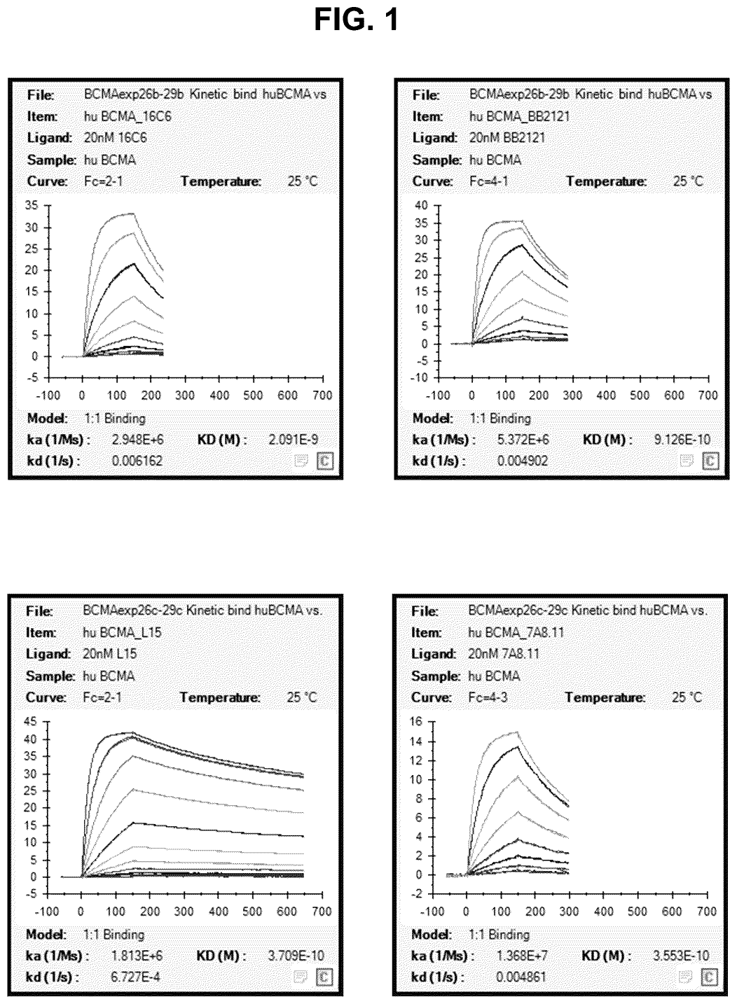

Candidate antibodies from the hybridoma screening described above were converted to scFv-Fc format, expressed recombinantly, and their target binding affinity was characterized by surface plasmon resonance (SPR). Briefly, anti-human IgG Fc-specific antibody was immobilized on a series S CM5 sensor chip (Cytiva, Marlborough, MA, USA) to allow for subsequent capture of candidate scFv-Fc molecules. To measure kinetics of BCMA binding, recombinant human BCMA (Acro Biosystems, Newark, DE USA) was flowed over the immobilized scFv-Fcs, prepared as described above, at a flow rate of 30 μL/min and at concentrations ranging from 0.03 nM to 30 nM. After 150 s, injection was stopped and dissociation was monitored for up to 400 s. Global fitting across multiple concentrations was used to determine kinetic parameters for binding as well as K_{D} values (FIG. 1). Clones 7A8.11 and L15 had higher affinities for human BCMA than Comparator I, with K_{D} values of 0.44 nM and 0.37 nM respectively.

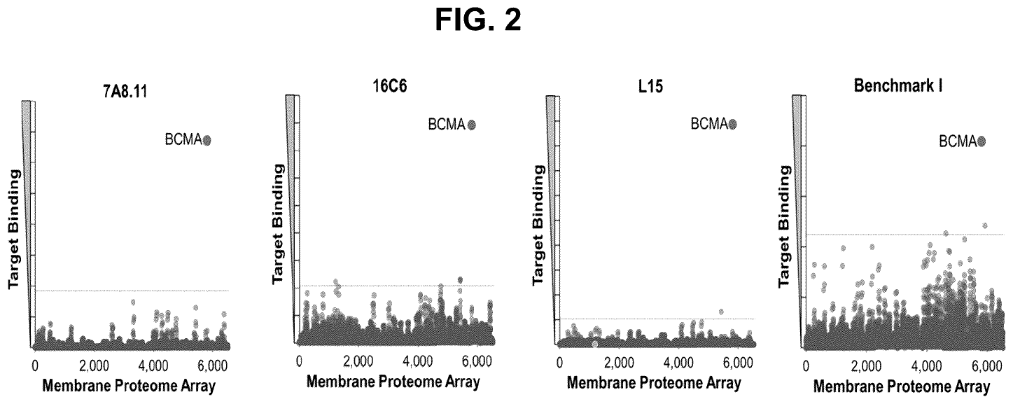

To assess the specificity of BCMA-binding scFvs, binding of scFv-Fc molecules to a 6,000 member membrane proteome array (Integral Molecular, Philadelphia, PA USA) was assessed. Briefly, cDNAs encoding for 6,000 unique human membrane proteins were individually transfected into HEK-293T cells and binding of BCMA-binding scFv-Fc molecules to these transfected cells was assessed using flow cytometry (FIG. 2). Off-target binding was validated with 293 cells transfected with plasmids encoding the identified target, protein A, or vector alone. 36 hours later, 4 fold serial dilutions of each ligand starting at 20 μg/ml were added to transfected cells to detect ligand binding using flow cytometry. Based on these results, no off-target binding was identified for any internally developed clones or Comparator Benchmark I (see US 2017/0226216).

### Example 2. CAR Conversion of Anti-BCMA Antigen Binding Fragments and Demonstration of Function in Primary Tcells

Of the prospective BCMA binding antibodies that were characterized, 10 were prioritized for conversion to scFv format and incorporated into CAR constructs for in vitro and in vivo assessments. To generate lentiviral expression vectors encoding BCMA-reactive CARs, BCMA scFvs were fused to a human IgG4P hinge, followed by human CD28 transmembrane domain, human 4-1BB intracellular domain and human CD3z intracellular domain in series from N- to C-terminus. The signal peptide from human CD33 was used to direct secretion and membrane insertion of the CAR. Sequence-verified constructs were used to generate lentivirus as described above.

Alternative CAR formats were also explored, including alternate hinges (CD8, CD28), transmembrane domains (CD8), costimulatory domains (CD28), and CD35 domains.

### CAR-T Manufacture

Primary T cells were activated with Dynabeads (Human T-Activator CD3/CD28) for 24 hours and then transduced with anti-BCMA CAR encoding lentiviruses. CAR expressing cells were identified and purified using recombinant BCMA protein labelled with AF647. CAR purity was confirmed via flow cytometry. Purified CAR-T cells were used for the below studies. In total, 10 unique scFv CARs constructs were tested against 3 benchmark controls.

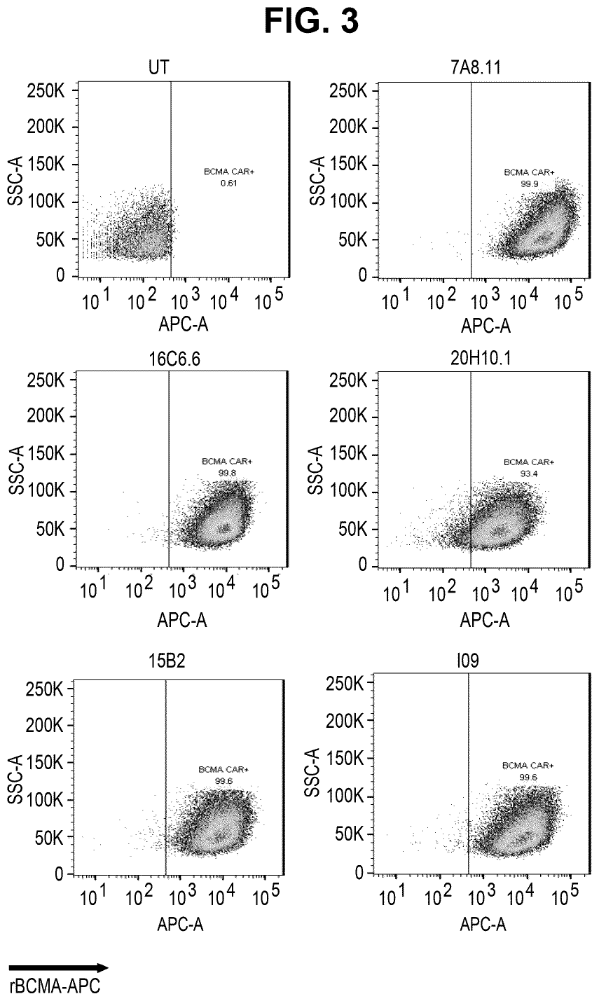

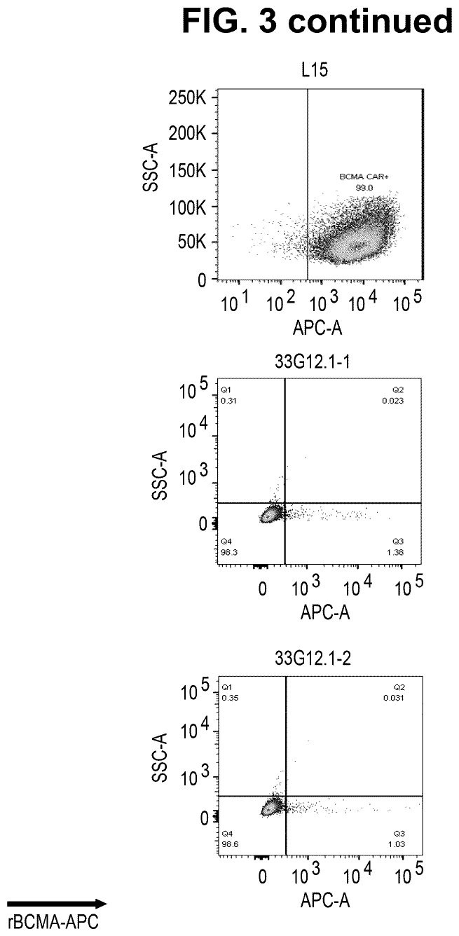

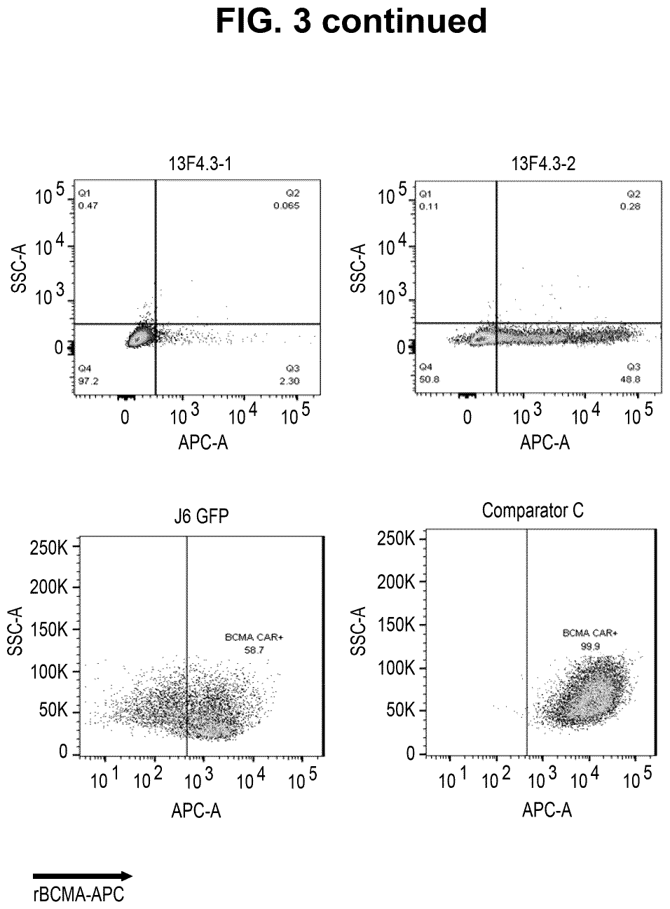

CAR-T cells were labelled with rBCMA conjugated with AF647 (BCMA-AF647) at 1 ug/ml, and fluorescence intensity was measured using flow cytometry. FIG. 3. Shows surface expression of each CAR clone. Clones 33G12.1-1, 33G12, 1-2, 13F4.3-1 failed to express CAR on the cell surface, while clone 7a8.11 had the brightest BCMA staining intensity and comparable expression to Comparator C (see WO 2018/028647).

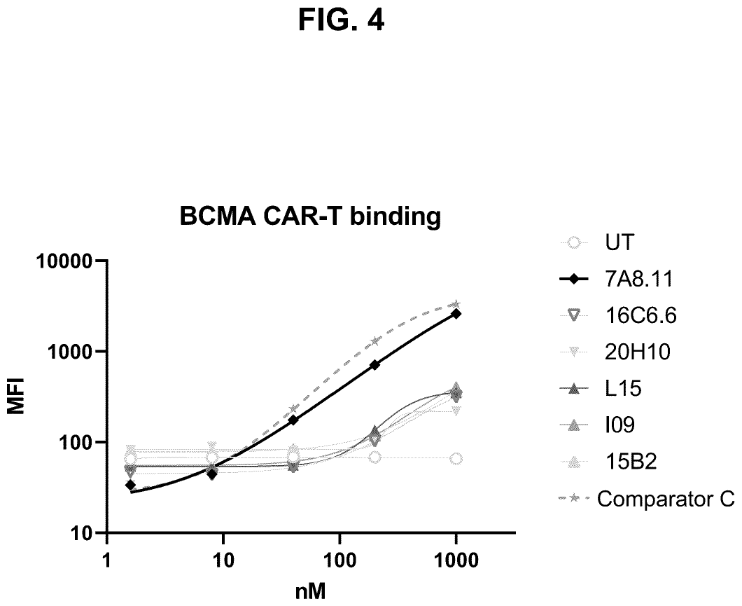

CAR-T cells were labelled with BCMA-AF647 at 0.3 nM-1000 nM concentrations of protein, and fluorescence intensity was measured via flow cytometry. FIG. 4 shows BCMA binding properties of each anti-BCMA CAR-T clone for soluble BCMA antigen. These binding curves demonstrate that the 7a8.11 clone has comparable BCMA binding to the comparator C benchmark.

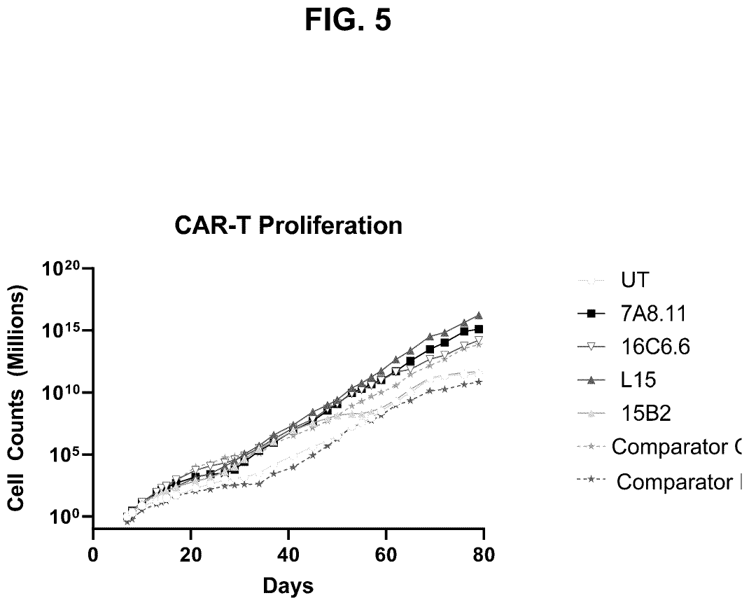

Using a representative T cell donor, expansion kinetics of the different BCMA CAR-T clones were tracked when cultured in AIM-V media containing human serum and IL2. FIG. 5. Shows cell viable cell counts taken every 2-3 days for 80 days to track expansion. FIG. 5 illustrates differences in growth kinetics and length of expansion of anti-BCMA CAR-T clones in primary T cells and highlights superior expansion profiles of the 7a8.11 CAR-T clone to Comparator C and Comparator I.

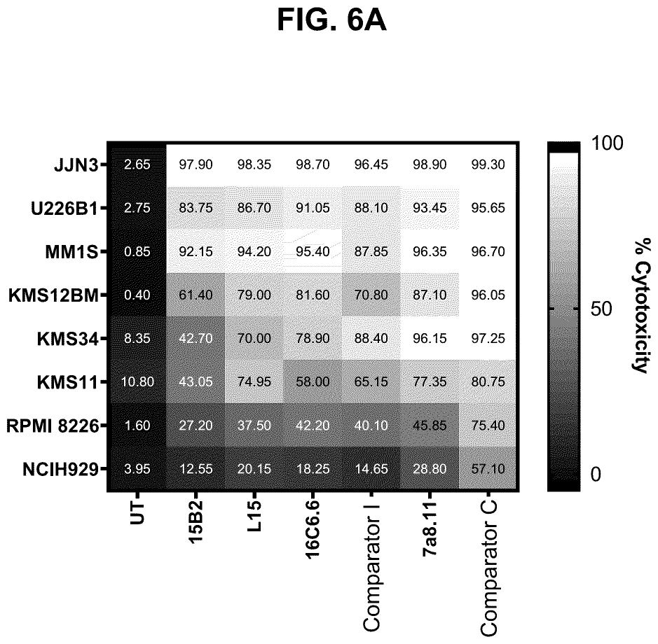

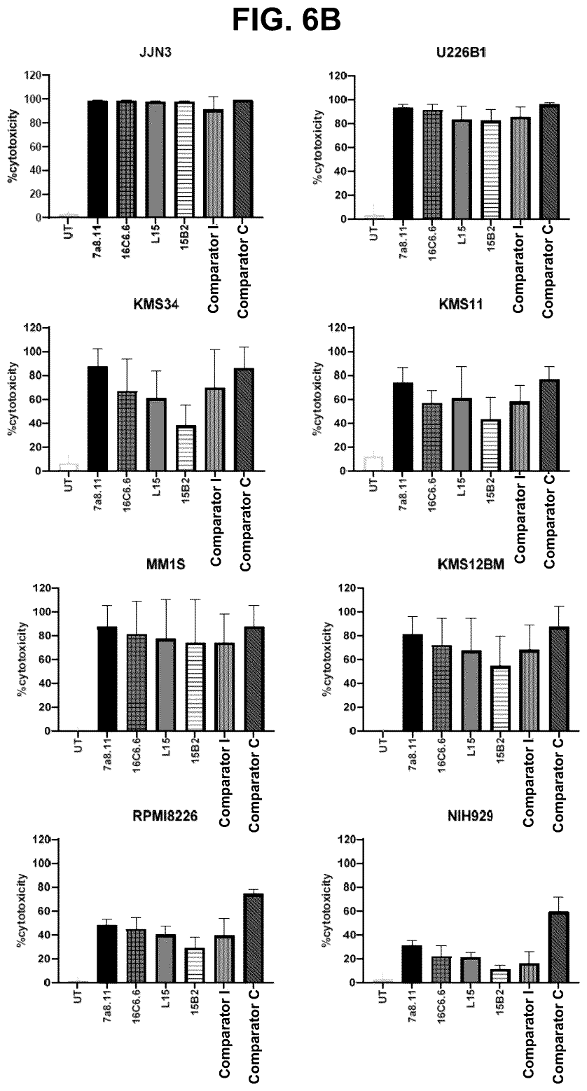

Assessment of CAR-T activity in vitro was carried out using multiple myeloma cell lines (JJN3, RPMI8226, U226B1, KMS11, KMS12, KMS34, NCIH929, MM1S) that were engineered to stably express luciferase. Briefly, cells were transduced with an mCherry/luciferase expressing lentivirus. mCherry positive cells were selected via FACS sort and pure populations of mCherry positive cells expressing luciferase were derived. Cells were then co-cultured with BCMA CAR-T cells from several donors at varying E:T ratios. Cytotoxicity of target multiple myeloma cells was evaluated 24 hours after co-culture by adding luciferin substrate and measuring luminescence by plate reader assay. FIGS. 6A and 6B are representative data showing cytotoxicity of target lines when co-cultured with each CAR-T clone and compared against two BCMA CAR-T benchmarks, Comparator I and Comparator C. FIG. 6A is a heat map with average % cytotoxicity of anti-BCMA CAR-T clones across 4 donors in 8 multiple myeloma cell lines after 24 hrs at 1:2 E:T ratio. FIG. 6B shows bar graphs of these same data with average % cytotoxicity and standard deviation from the 4 donors. All of the internally derived BCMA CAR-T clones demonstrate killing of multiple myeloma cells, and the 7a8.11 BCMA CAR-T had comparable cytoxic effect across all multiple mycoloma lines to both Comparator I and Comparator C. More specifically, clone 7a8.11 has greater cytotoxicity than Comparator I and only slightly reduced cytotoxicity compared to Comparator C in three cell lines.

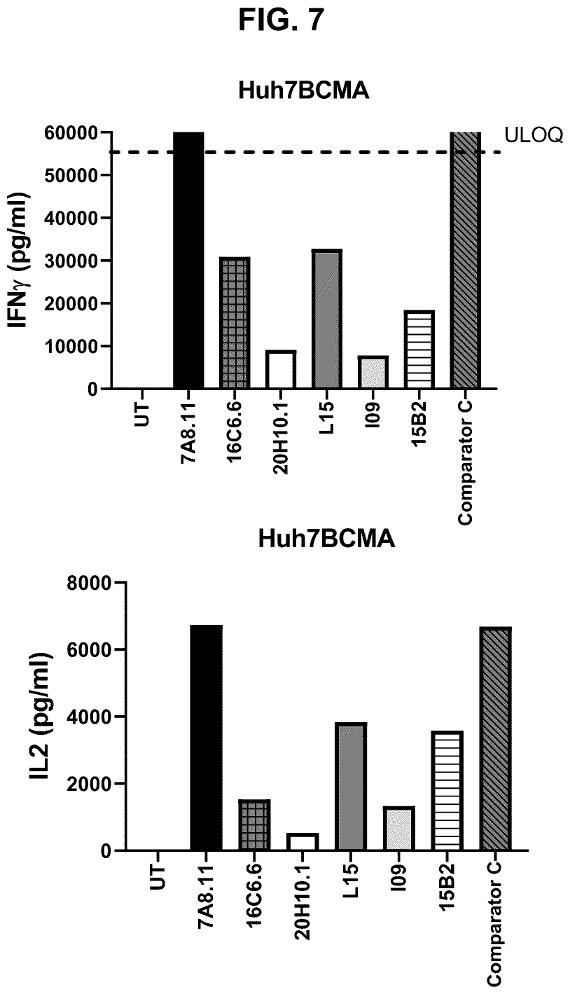

To measure effector cytokine production, Huh7 target cells were engineered to express BCMA antigen, and parental cells were used as negative controls. Briefly, Huh7 cells were transfected with a lentivirus containing TNFRSF17 and a puromycin resistance cassette, and cells successfully integrating the gene were selected based on puromycin resistance. Huh7BCMA target cells were then co-cultured with anti-BCMA CAR-T clones at varying E:Ts, and supernatants were collected at 24 hours to quantify IFNy and IL2 production via Meso Scale Discovery assay (MSD). FIG. 7 illustrates representative effector cytokine production from anti-BCMA CAR-T cells when co-cultured with a BCMA-expressing huh7 engineered cell line at an E:T ratio of 1:2. All of the anti-BCMA CAR-T cells show effector cytokine production in response to CAR-T cell activation and killing. The 7a8.11 clone shows the highest level of cytokine production in response to antigen, which is comparable to IFNy and IL2 levels produced by Comparator I under the same conditions.

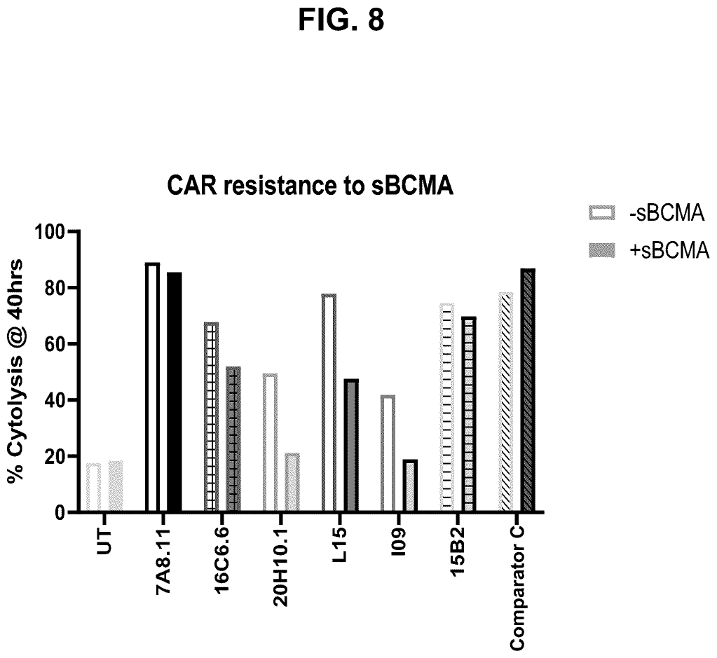

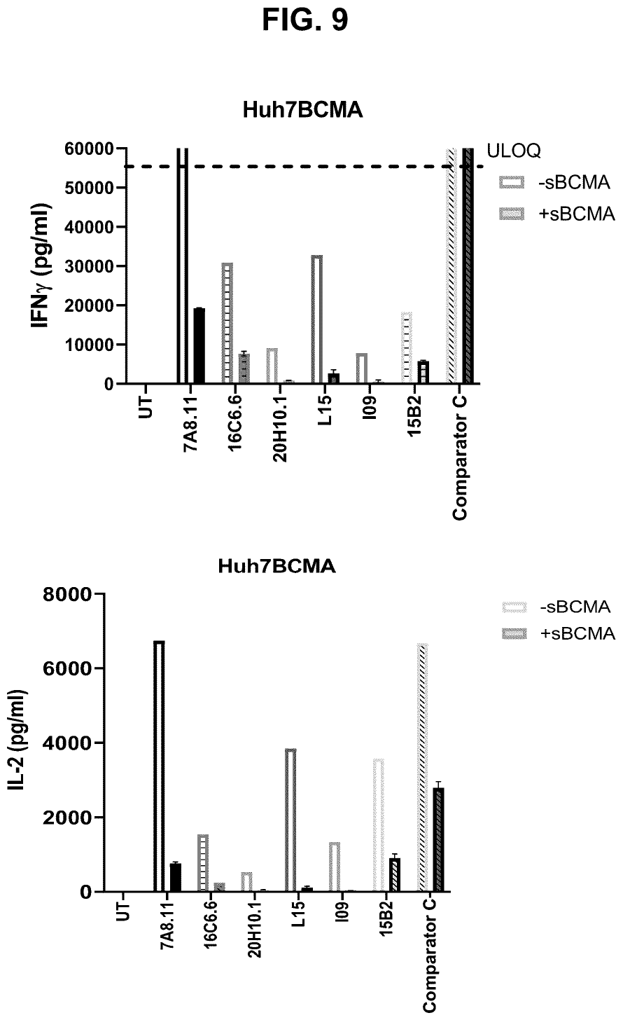

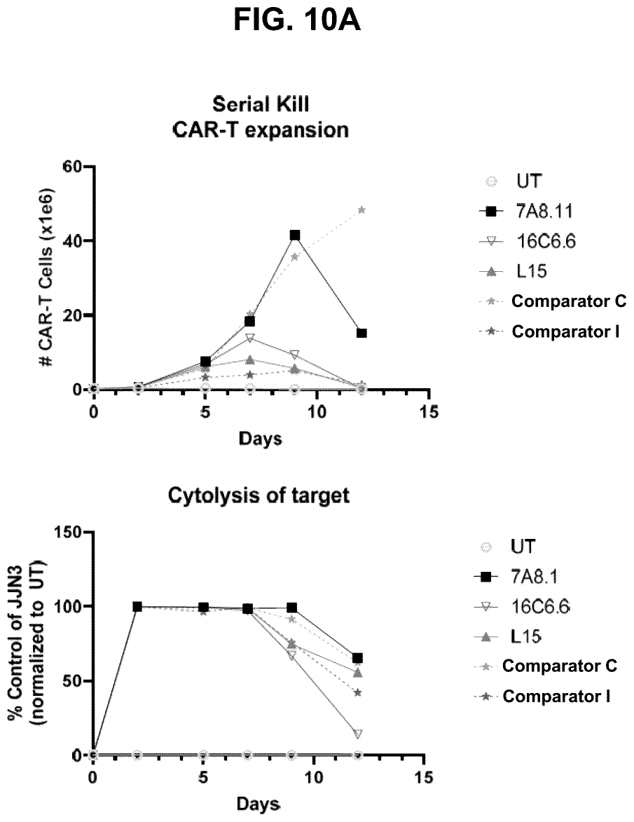

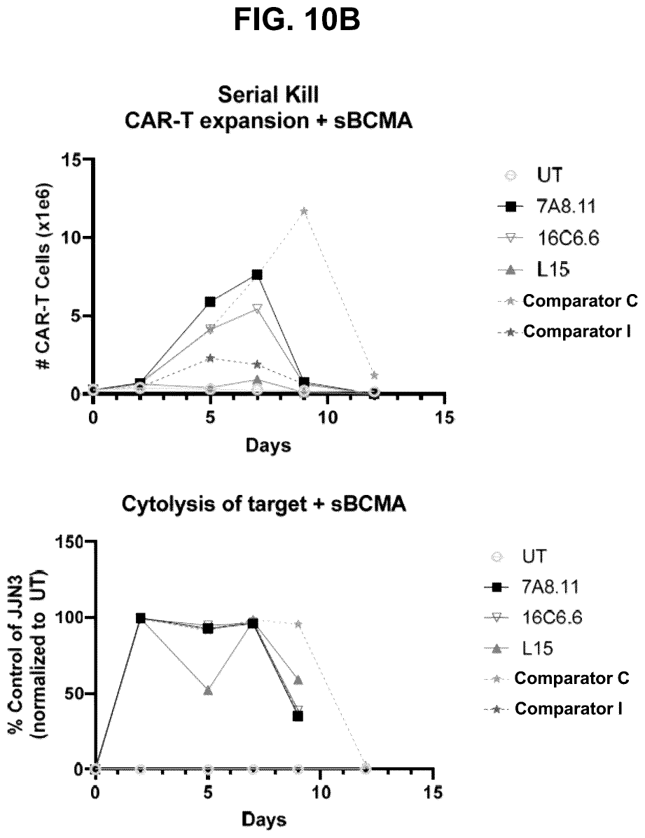

To determine the effects of soluble BCMA on CAR-T killing, Huh7 BCMA-expressing cells were seeded on RTCA E-Plates (Agilent) for impedence based measurement of cytotoxicity by xCELLigence. Soluble recombinant BCMA protein (10 ug/ml) was added to the cells followed by co-culture with BCMA CAR-T cells. FIG. 8 illustrates differences in % cytolysis observed at 40 hrs post co-culture with BCMA-expressing huh7 cells in the presence or absence of soluble BCMA protein. These data demonstrate that the 7A8.11 BCMA CAR-T cells have the highest level of cytolysis across all BCMA CAR-T clones and also show equivalent levels of cytolysis to Comparator C in the presence and absence of soluble protein. From this co-culture, supernatants were collected at 24 hours post addition of CAR-T cells, and IFNy and IL-2 levels were measured by MSD to compare differences in effector cytokine production in the presence and absence of soluble BCMA antigen in the culture. FIG. 9. demonstrates various effects of soluble BCMA protein on effector cytokine production. All BCMA CAR-T cells evaluated displayed lower levels of IFNy and IL2 production in the presence of soluble antigen; however the 7A8.11 CAR-T clone was least negatively impacted by the presence of soluble protein relative to other internally developed CAR clones. In order to evaluate differences in antigen-driven CAR-T expansion and persistence of the cells, an in vitro serial kill experiment was performed in which CAR-T cells were repeatedly co-cultured with the multiple myeloma target cell line, JJN3, at an E:T ratio of 1:1. Every 2-3 days, co-cultures were sampled and evaluated for T-cell and tumor cell counts and viability by flow cytometry. Following this determination, CAR-T/tumor cell co-cultures were then “fed” viable JJN3 cells to return the co-culture to a 1:1 E:T ratio. FIG. 10A shows CAR-T expansion of each clone over a 12 day serial kill experiment (top panel) and % cytolysis of target JJN3 cells after each round of co-culture as a measure of CAR-T cell function and persistence (bottom panel). FIG. 10A shows 7A8.11 CAR-T cells have superior antigen-driven expansion and persistence based on the duration of expansion and prolonged cytolysis of the targets over other BCMA CAR-T clones. Furthermore, 7A8.11 CAR-T cells demonstrated expansion and functional persistence on par with Comparator C and superior to Comparator I. FIG. 10B shows persistence and expansion of CAR-T cells (top panel) and cytolysis of target cells (bottom panel) following repeat antigen stimulation in the presence soluble BCMA. FIG. 10B similarly evaluates antigen-dependent CAR-T cell expansion and functional persistence by JJN3 co-culture in the presence of soluble recombinant BCMA protein. Compared to expansion in the absence of soluble BCMA (FIG. 9A), expansion of all CAR-Ts was reduced and had shortened persistence. However, 7A8.11 CAR-T cells remained superior in expansion compared to other internal clones and Comparator I. Based on both sets of data from FIGS. 10A and 10B, 7A8.11 CAR-T cells demonstrate favorable functional persistence and expansion in response to antigen and comparable functionality to comparator benchmarks, Comparator C and Comparator I.

### In Vivo Animal Studies:

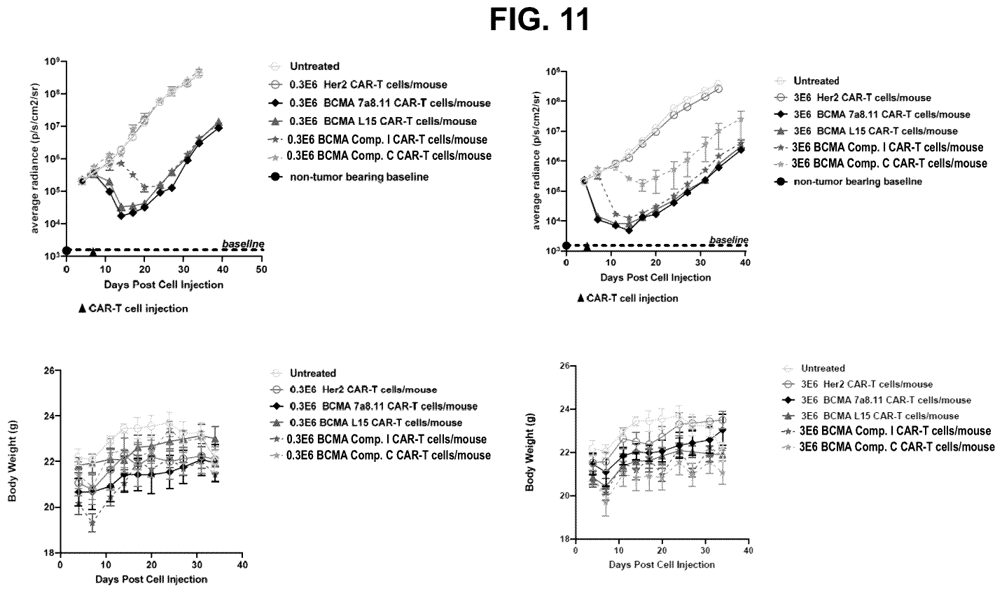

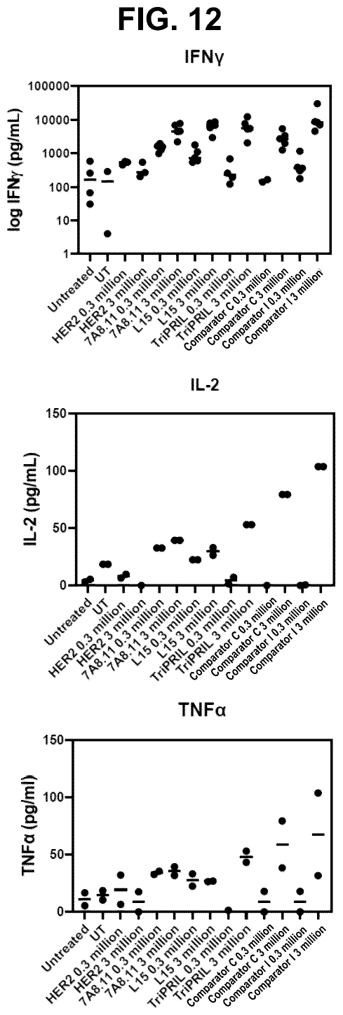

All animal experiments were conducted in a facility accredited by the Association for Assessment of Laboratory Animal Care (AALAC) under Institutional Animal Care and Use Committee (IACUC) guidelines and appropriate animal research approval. To evaluate in vivo BCMA CAR-T function in an in vivo disseminated model of multiple myeloma, we infused 10e6 multiple myeloma 1S-luc cells intravenously into NSG mice. Four days post tumor cell infusion, mice were imaged and then infused intravenously with either 0.3e6 or 3e6 CAR-T cells. Animals were weighed and imaged (via intraperitoneal injection of luciferin) every 3-4 days to look for signs of morbidity and track tumor cell growth. FIG. 11 shows in vivo tumor control of anti-BCMA CAR-T clones and clinical benchmarks at both high and low doses of CAR-T cells in a disseminated multiple myeloma model (MM1.S). At both high and low doses, 7A8.11 CAR-T cells demonstrate better tumor control over either comparator benchmarks (Comparator C and Comparator I) and is functionally equivalent to the L15 internally derived clone. Furthermore, administration of CAR-T cells did not demonstrate any treatment related toxicities, such as cytokine release syndrome (CRS), at these dose levels. Three days post CAR-T infusion, animals were bled and serum cytokine levels were quantified using MSD. FIG. 12 demonstrates dose responsive effector cytokine production by BCMA CAR-T clones in the in vivo MM1.S tumor model.

### Example 3. BCMA CAR-T_{REX} Cells

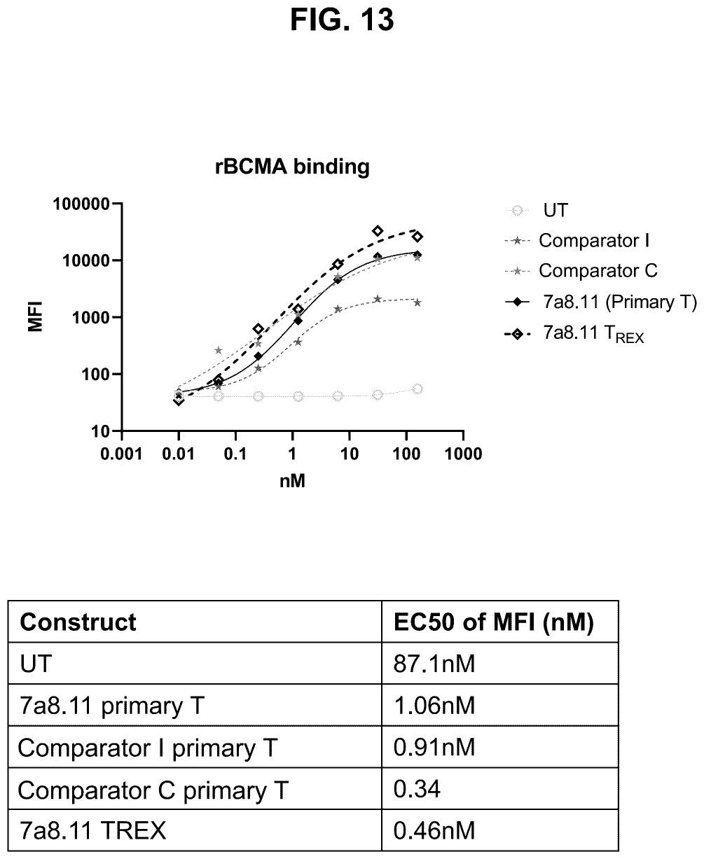

To characterize BCMA binding on the surface of 7A8.11 CAR expressed in the T_{REX} chassis, CAR-T cells were labelled with BCMA-AF647 at 0.3 nM-1000 nM concentrations of protein and fluorescence intensity was measured via flow cytometry. FIG. 13 shows BCMA binding curves of 7A8.11 CAR-T_{REX} compared to primary 7A8.11 CAR-T cells and benchmarked against Comparators I and C. The 7A8.11 CAR expressed in T_{REX} cells demonstrates the highest capacity for BCMA binding on the cell surface compared to 7A8.11 CAR expressed in primary T cells and Comparators I and C. Moreover, the EC50 of 7A8.11 in T_{REX} is higher than when expressed in primary T cells, more than Comparator I, and similar to Comparator C.

### Example 4. In Vitro Cytotoxicity of BCMA CAR-T_{REX} Cells in Multiple Myeloma Cell Lines

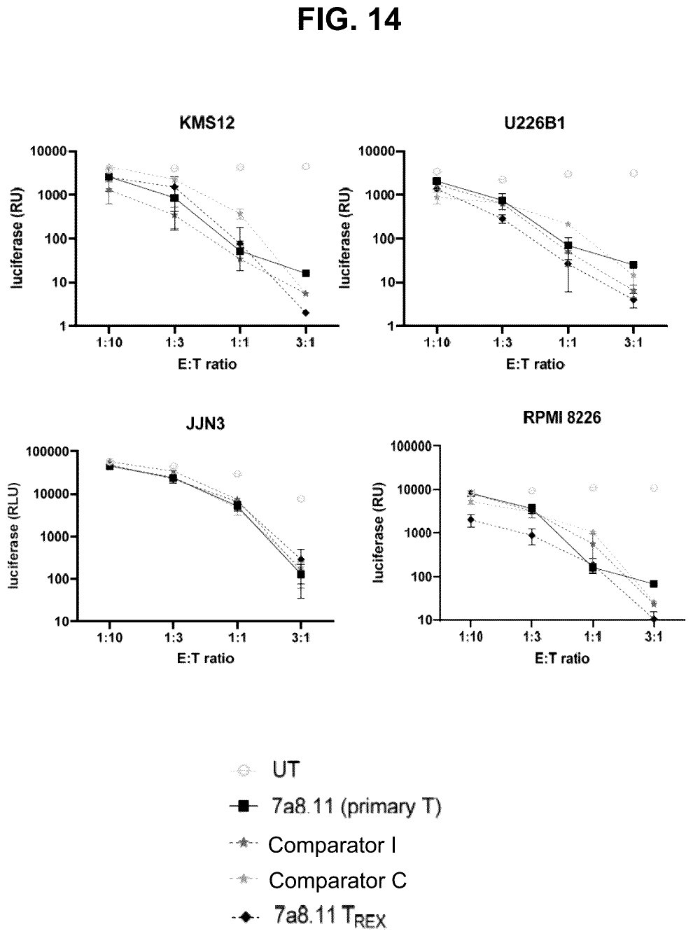

CAR-Ts were seeded at 4 different E:T ratios with each of the following luciferase expressing target lines: KMS12, U226B1, JJN3, and RPMI8226. Cytotoxicity was measured 24 hours after CAR-T addition and viability was measured following the addition of luciferin substrate. Luminescence from viable cells was measured via plate reader. 7A8.11 CAR-T_{REX} cells displayed comparable cytotoxic function to primary T cells expressing the 7A8.11 CAR and to Comparators I and C across all E:T ratios and for all target lines (see FIG. 14).

### Example 5. Cytokine Profile of BCMA CAR-T_{REX} Cells

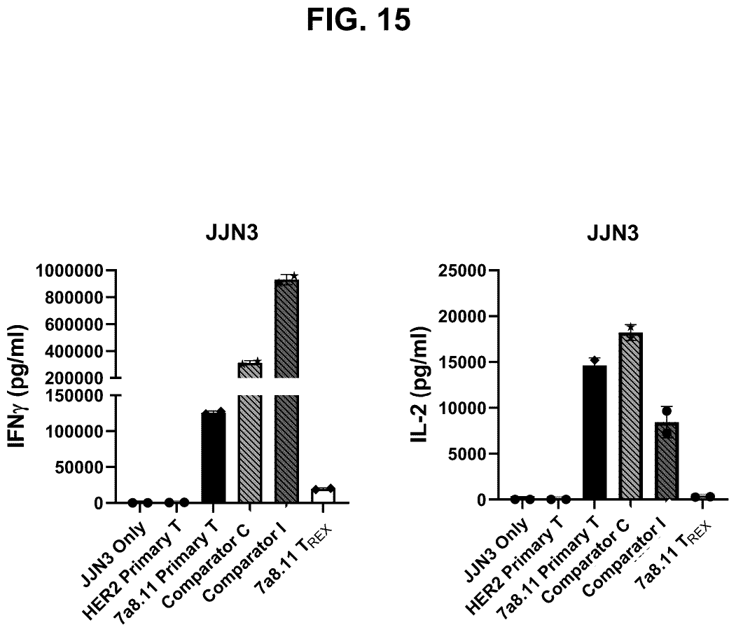

FIG. 15 shows an example of effector cytokine profile from 7A8.11 CAR-T_{REX} cells when co-cultured with the target cell line, JJN3, and compared to primary T cells expressing 7A8.11 and Comparator benchmarks I and C. CAR-T cells were seeded at an E:T ratio of 1:1 with JJN3, and supernatants were sampled 24 hours post co-culture to measure cytokine production of IFNy and IL2 by MSD. 7A8.11 CAR-T_{REX} cells demonstrate between 2-15% effector cytokine production compared to 7A8.11 primary T cells and less than 5% effector cytokine production of the clinical comparators, suggesting a potentially safer cytokine profile with 7A8.11 CAR-T_{REX} cells.

### Example 6. In Vivo Tumor Control of Multiple Myeloma Cells by BCMA CAR-T_{REX} Cells

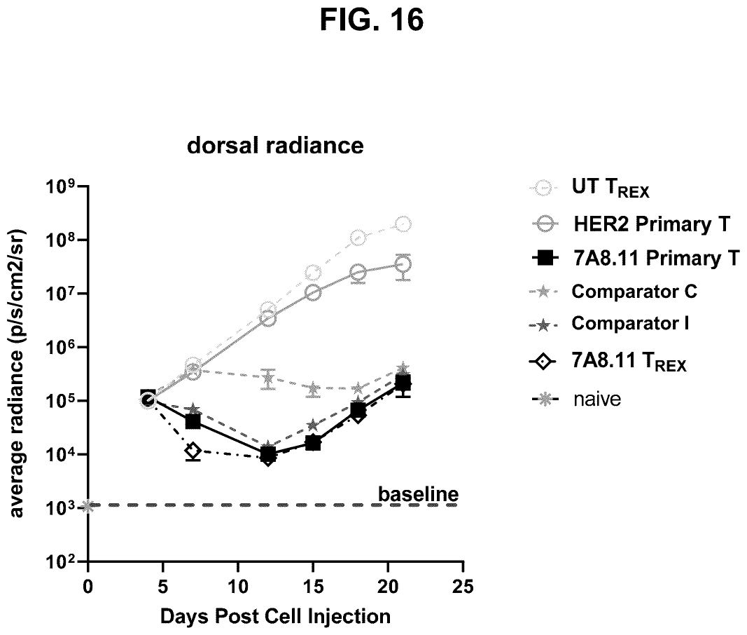

FIG. 16 shows in vivo tumor control of MM1.S cells by 7A8.11 CAR-T_{REX} cells compared to clinical benchmarks, Comparators I and C. A non-targeting HER2 CAR-T was used as control. In this in vivo disseminated model of multiple myeloma, 10e6 MM1S-luc cells were infused intravenously into NSG mice. Four days post tumor cell infusion, mice were imaged and then infused intravenously with 7A8.11 CAR-T_{REX} cells or 7A8.11 primary T cells or with Comparators I and C. Animals were weighed and imaged (via intraperitoneal injection of luciferin) every 3-4 days to look for signs of morbidity and track tumor cell growth. FIG. 16 illustrates identical tumor clearing kinetics of 7A8.11 CAR-T_{REX} cells with 7A8.11 primary T cells and the same or better tumor clearing kinetics relative to Comparators I and C.

### Example 7. Daratumumab Treatment Protects Anti-BCMA-T_{REX} Cells from NK Cells

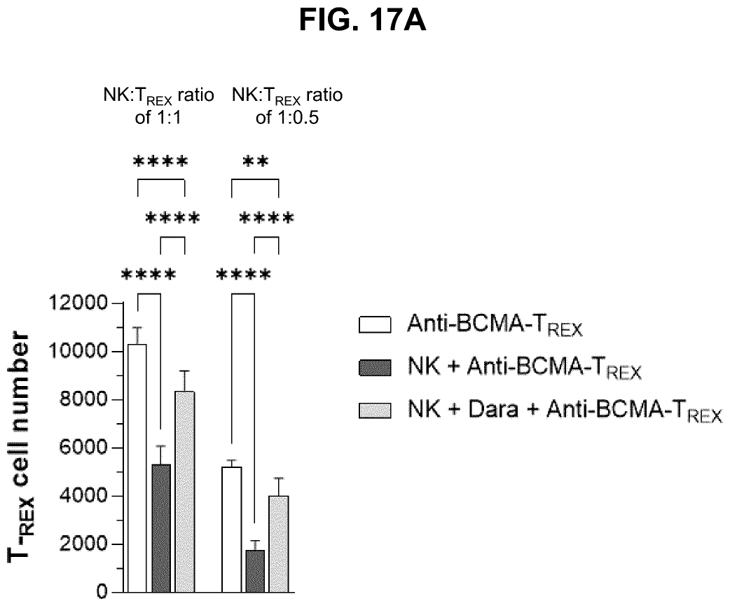

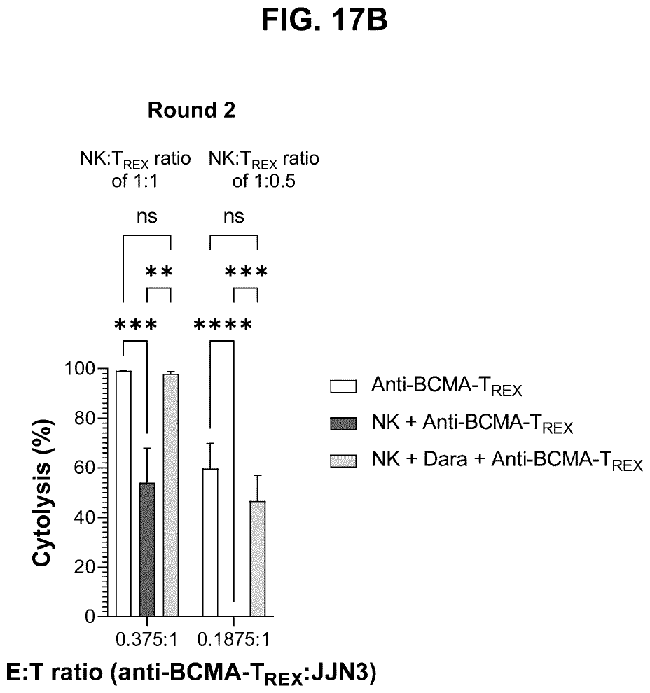

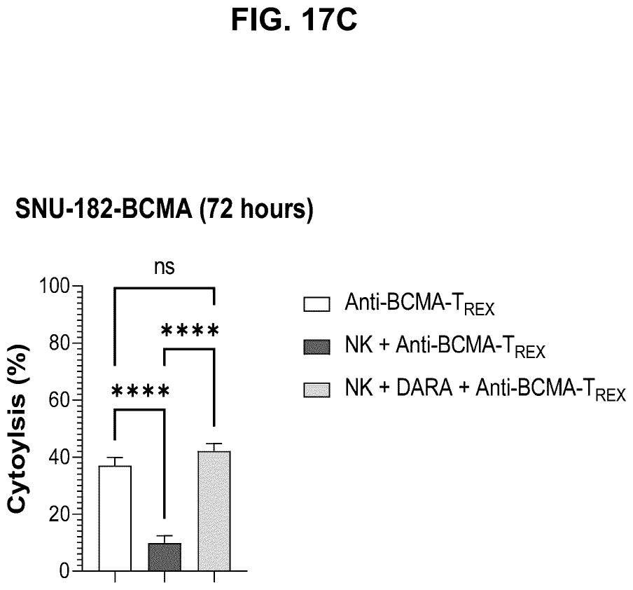

FIG. 17 shows that Daratumumab (Dara) treatment protects anti-BCMA-T_{REX} cells from NK cells, and the remaining T_{REX} cells are functional. Briefly, purified NK cells were cultured overnight (NK-Xpander medium, 500 IU/mL IL-2) in the presence or absence of Dara (10 ug/mL). The next day, NK cells were washed and co-cultured with anti-BCMA-T_{REX} cells at an NK: T_{REX} ratio of 1:1 or 0.5:1 for 5 hours. After 5 hours, co-cultures were assessed by flow cytometry to quantify NK cell and anti-BCMA-T_{REX} cell numbers, demonstrating Dara-mediated protection of anti-BCMA-T_{REX} cell numbers (FIG. 17A). Cells were further progressed through two rounds of serial kill of JJN3 target cells at indicated E:T ratios, with % cytolysis of tumor cells measured by a luciferase assay (FIG. 17B); or a single round of cell killing with a BCMA-ectopically expressing SNU-182 adherent cell line, with tumor cell killing evaluated by Xcelligence (FIG. 17C). The data are representative of studies performed with NK cells from three different donors. As seen in FIG. 17, Dara treatment enables protection of anti-BCMA-T_{REX} cell numbers, while maintaining the cytotoxic capacity of these cells.

### Example 8. Cryo-Recovered BCMA CAR-T_{REX} Cells Demonstrate In Vivo Tumor Clearance

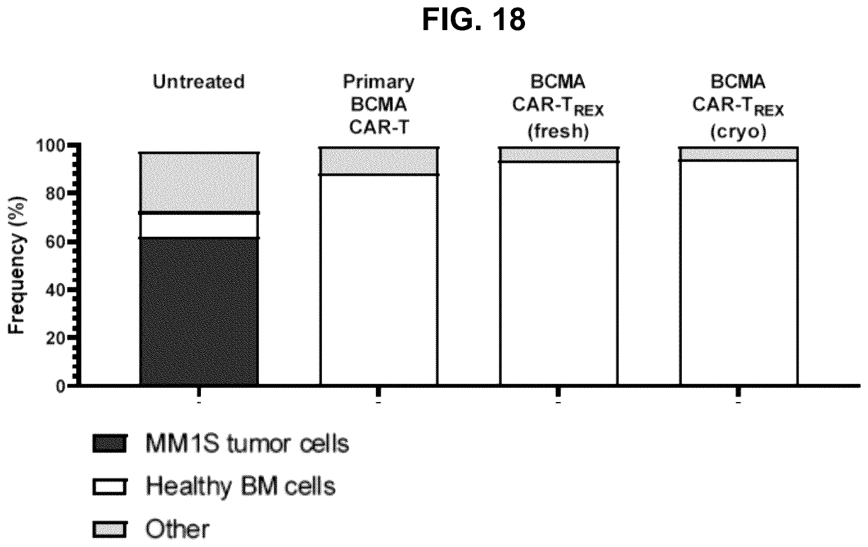

NSG mice were inoculated with 10E6 MM1S-luciferase tumor cells. 3 days later, Primary BCMA CAR-T cells (7A8.11 CAR-T cells), BCMA CAR-T_{REX} cells (7A8.11) that were cryo-recovered and cultured for optimal activity (fresh), or BCMA CAR-T_{REX} cells (7A8.11) that were administered immediately following cryo-recovery (cryo) were dosed at 10E6 cells per mouse. 25 days after MM1S cell administration, bone marrow was harvested from mice and analyzed for the presence of MM1S tumor cells, healthy bone marrow cells, or other populations. Cryo-recovered and immediately administered BCMA CAR-T_{REX} cells showed comparable tumor clearance and murine bone marrow restoration as other groups (see FIG. 18).

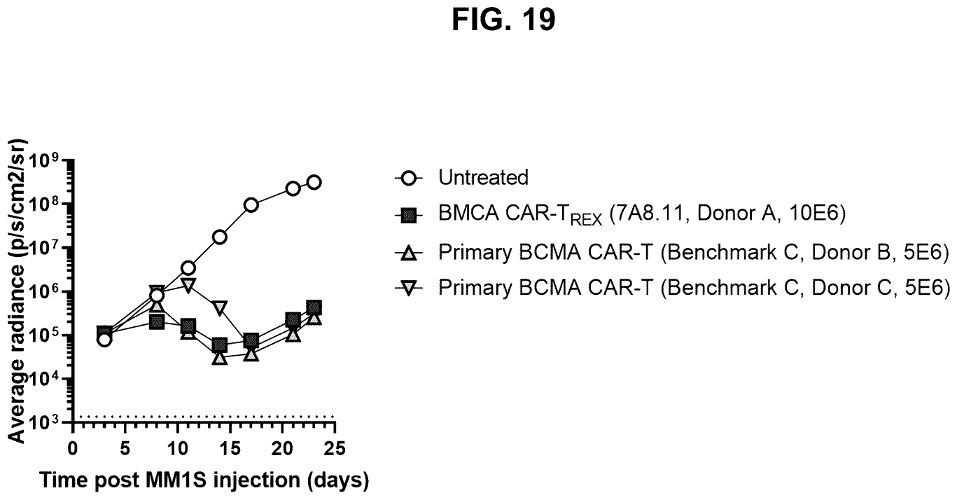

NSG mice were inoculated with 10E6 MM1S-luciferase tumor cells. 3 days later, Primary BCMA CAR-T cells (Benchmark C CAR-T cells) from two donors or BCMA CAR-T_{REX} cells (7A8.11) were administered immediately following cryo-recovery (cryo) at indicated doses. Tumor burden was monitored twice weekly using IVIS imaging. BCMA CAR-T_{REX} cells demonstrated deep in vivo tumor clearance when dosed immediately following cryo-recovery (see FIG. 19).

### Example 9. BCMA Antigen Density and Cell Composition Between SLE and Healthy Donors

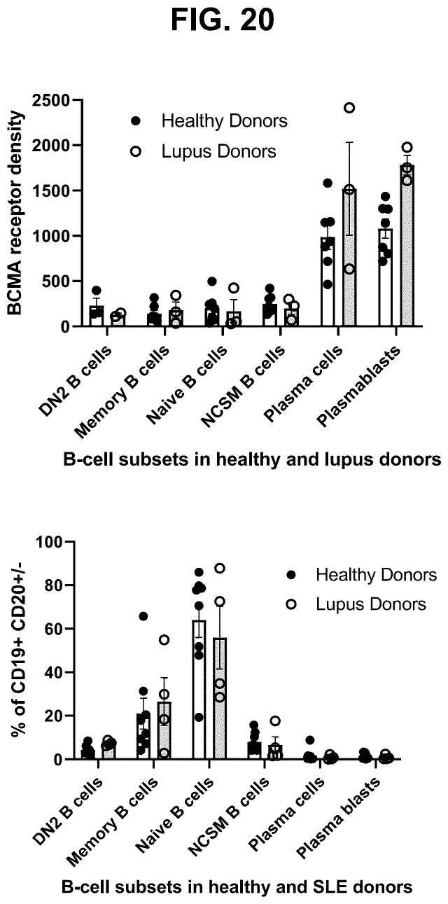

The ability of BCMA-targeting cells to deplete BCMA+ cells is thought to be dependent on the antigen density on the surface of cells. Accordingly, the BCMA antigen density and cell composition of patients with systemic lupus erythematosus (SLE) was compared to healthy donors. PBMCs were isolated from fresh whole blood from patients with SLE and healthy donors, and the PBMCs were assessed for BCMA receptor density and B cell subset percentages. As shown in FIG. 20, the cellular make-up of the B cell compartment was broadly similar between healthy donors and SLE patients. Moreover, target-expression (BCMA receptor density) was also similar between healthy donors and SLE patients (or slightly higher for SLE patients; especially on plasmablasts).

### Example 10. BCMA CAR-T Cells Deplete Target-Expressing Healthy Human Plasma Cells to a Similar Extent as MM1S (BCMA+) Tumour Cells

BCMA CAR-T cell research lots were manufactured from fresh peripheral blood of individual healthy donors. Briefly, healthy donors' PBMC were harvested from blood by Ficoll gradient centrifugation, and CD4 and CD8 positive T cells were enriched from the leukocyte fraction. Isolated T cells were then activated, transduced with 7A8.11 BCMA lentiviral vector, and expanded in culture flasks before cells were washed, harvested and frozen in a cryopreservation medium.

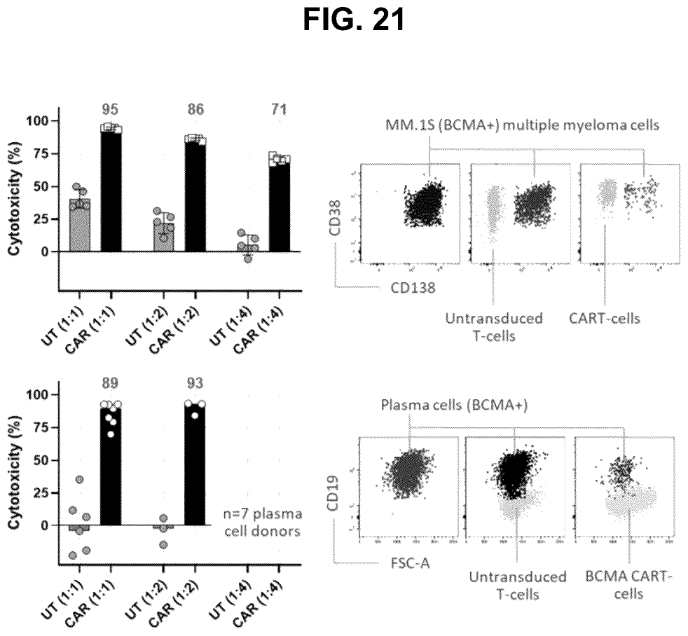

Primary human plasma cells (CD138+selection) were isolated from fresh whole blood of healthy donors. The plasma cells were then co-cultured with the 7A8.11 BCMA CAR-T cells or un-transduced T cells (untargeted) at varying effector:target ratios. As a control, MM1S cells (a multiple myeloma cell line expressing BCMA) were also co-cultured with the product or untransduced T cells at varying effector:target ratios. As shown in FIG. 21, BCMA CAR-T cells were able to deplete the primary human plasma cells or MM1S tumour cells to similar degrees (89% vs 95% at an effector:target of 1:1 and 93% vs 86% at an effector:target 1:2 of, respectively), while the untransduced T cells mediated minor depletion of either the primary human plasma cells or the MM1S tumour cell line.

### Example 11. In Vitro Differentiated Plasma Blasts from SLE and Healthy Donors Shows a Dose Dependent Depletion (E:T) by BCMA CAR-T Cells

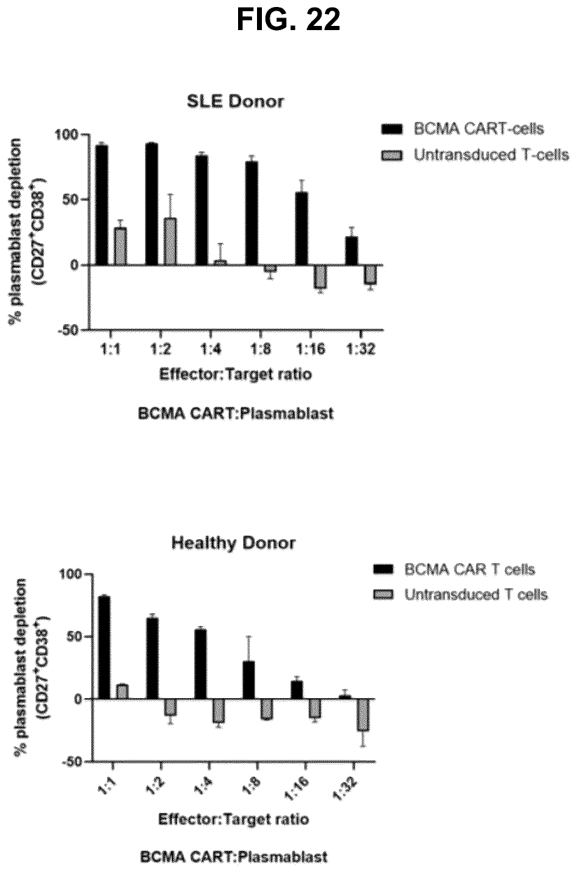

Primary human naïve B-cells (IgD+CD27-selection) were isolated from fresh or frozen PBMCs from healthy or SLE donors. The naïve B-cells were then differentiated using a proprietary mixture of cytokines to drive plasmablast differentiation (BCMA+B-cells). After 5 days of differentiation, the cells were then co-cultured with the 7A8.11-transduced CAR-T cells or un-transduced T cells (untargeted) at varying effector:target ratios. The 7A8.11-transduced CAR-T cells were able to deplete the healthy or SLE primary human differentiated plasmablasts to similar extents (see FIG. 22).

### Example 12. BCMA Targeting CAR-T Cells Reduced BCMA+ Cells in Xenogeneic Model of Graft Versus Host Disease

In order to assess the ability of BCMA-targeted CAR-T cells to deplete primary B cells expressing BCMA in vivo, a pharmacodynamic (PD) study was performed in the xenogencic model of graft versus host disease (XenoGvHD). To prepare for this XenoGvHD PD study, healthy human donor PBMC was isolated from fresh leukopak (StemExpress) and stored frozen until the engraftment. Autologous un-transformed T cells (UTT) or autologous BCMA targeted CAR-T cells (BCMA CAR-T) were manufactured from the PBMC of the same donor. Briefly, isolated T cells were activated, transduced with 16C6 BCMA lentiviral vector, and expanded in culture flasks before cell were washed, harvested and frozen in a cryopreservation medium.

On Day-1, 8-10 week old female NOD scid gamma mice (NSG; Jackson Laboratories) were pre-conditioned with sub-lethal whole body irradiation (1 Gy). On Day 0, 15 million healthy human donor total PBMC was injected intravenously. Four hours later, mice were injected intravenously with either phosphate buffered saline (PBS), 3 million autologous UTT cells, or 3 million autologous BCMA CAR-T cells (n=5 mice/group). On Day 12, mice were euthanized and tissues were collected for FACS analysis to determine whether or not these BCMA CAR-T cells depleted the BCMA+ cells observed to be present in this in vivo model.

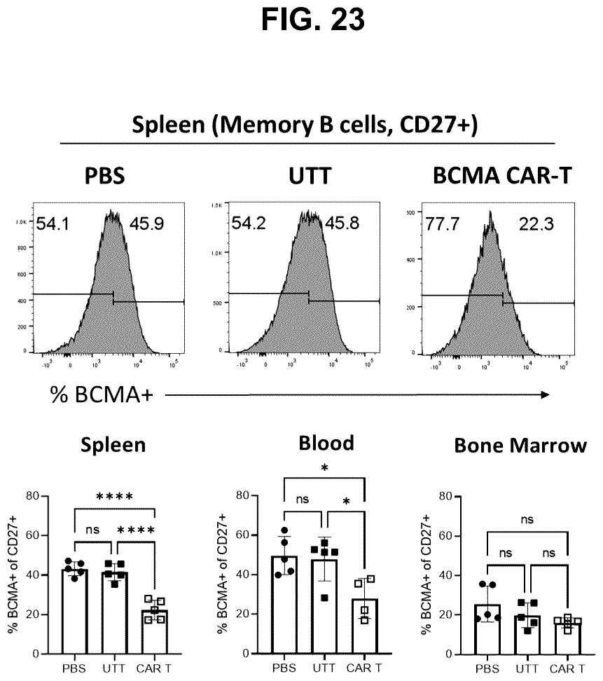

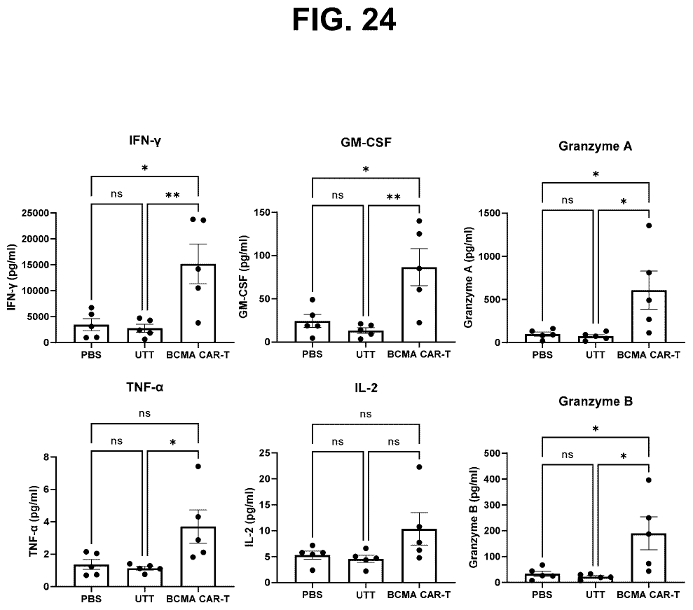

Mice treated with the BCMA CAR-T product showed significantly reduced percentage of CD27+ memory B cells expressing BCMA compared to PBS and UTT treated mice in the spleen and whole blood (FIG. 23). Serum cytokines and cytolytic granzymes generally associated with CAR-T therapy were assessed using a multiplex ELISA from Meso-scale Discovery and were found to be significantly elevated in mice treated with BCMA targeting CAR-T cells compared to PBS and UTT treated mice (FIG. 24).

Sequences.

| SEQ | | |
|---|---|---|
| ID | Description | Sequences |
| 1 | 7A8.11 HC | EVQLVESGGGLVQPGGSLRLSCEASGFTFSSFWMNWVRQAPGKG |
| | | LEWVANIKEDGSEKYYVDSVKGRFTISRDNAKNSLYLQMNSLRV |
| | | EDTAVYYCARALDYYGMDVWGQGTTVTVSS |
| 2 | 7A8.11 HC CDR 1 | GFTFSSFWMN |
| 3 | 7A8.11 HC CDR 2 | NIKEDGSEKYYVDSVKG |
| 4 | 7A8.11 HC CDR 3 | ALDYYGMDV |
| 5 | 7A8.11 LC | YIVMTQTPLSLPVTPGEPASISCRSSQSLLDSDDGNTYLDWYLQ |
| | | KPGQSPQLLIYTLSYRASGVPDRFSGSGSGTDETLKISRVEAED |
| | | VGVYYCMQRIEFPSYTFGQGTKLEIK |
| 6 | 7A8.11 LC CDR 1 | RSSQSLLDSDDGNTYLD |
| 7 | 7A8.11 LC CDR 2 | TLSYRAS |
| 8 | 7A8.11 LC CDR 3 | MQRIEFPSYT |
| 9 | 7A8.11 scFv | MPLLLLLPLLWAGALAEVQLVESGGGLVQPGGSLRLSCEASGFT |
| | | FSSFWMNWVRQAPGKGLEWVANIKEDGSEKYYVDSVKGRFTISR |
| | | DNAKNSLYLQMNSLRVEDTAVYYCARALDYYGMDVWGQGTTVTV |
| | | SSGGGGSGGGGSGGGGSGGGGSYIVMTQTPLSLPVTPGEPASIS |
| | | CRSSQSLLDSDDGNTYLDWYLQKPGQSPQLLIYTLSYRASGVPD |
| | | RFSGSGSGTDFTLKISRVEAEDVGVYYCMQRIEFPSYTFGQGTK |
| | | LEIK |
| 10 | 33G12.1-1 HC | QVQLQESGPGLVKPSETLSLTCTVSGGSISSYYWSWIRQPAGKG |
| | | LEWIGRIYTSGSTNYNPSLKSRVTMSVDTSKNQFSLKLNSVTAA |
| | | DTAVYYCARSGWDNSGSYYSDAFDIWGQGTMVTVSS |
| 11 | 33G12.1-1 HC CDR 1 | GGSISSYYWS |
| 12 | 33G12.1-1 HC CDR 2 | RIYTSGSTNYNPSLKS |
| 13 | 33G12.1-1 HC CDR 3 | SGWDNSGSYYSDAFDI |
| 14 | 33G12.1-1 LC | QPVLTQPPSASASLGASVTLTCTLSSGYSNYKVDWYQQRPGKGP |
| | | RFVMRVGTGGIVGSKGDGIPDRFSVLGSGLNRYLTEKNIQEEAE |
| | | SDYHCGADHGSGSNFVWVFGGGTRLTVL |
| 15 | 33G12.1-1 LC CDR 1 | TLSSGYSNYKVD |
| 16 | 33G12.1-1 LC CDR 2 | VGTGGIV |
| 17 | 33G12.1-1 LC CDR 3 | GADHGSGSNFVWV |
| 18 | 33G12.1-1 scFv | MPLLLLLPLLWAGALAQVQLQESGPGLVKPSETLSLTCTVSGGS |
| | | ISSYYWSWIRQPAGKGLEWIGRIYTSGSTNYNPSLKSRVTMSVD |
| | | TSKNQFSLKLNSVTAADTAVYYCARSGWDNSGSYYSDAFDIWGQ |
| | | GTMVTVSSGGGGSGGGGSGGGGSGGGGSQPVLTQPPSASASLGA |
| | | SVTLTCTLSSGYSNYKVDWYQQRPGKGPRFVMRVGTGGIVGSKG |
| | | DGIPDRFSVLGSGLNRYLTEKNIQEEAESDYHCGADHGSGSNFV |
| | | WVFGGGTRLTVL |
| 19 | 33G12.1-2 HC | QVQLQESGPGLVKPSETLSLTCTVSGGSISSYYWSWIRQPAGKG |
| | | LEWIGRIYTSGSTNYNPSLKSRVTMSVDTSKNQFSLKLNSVTAA |
| | | DTAVYYCARSGWDNSGSYYSDAFDIWGQGTMVTVSS |
| 20 | 33G12.1-2 HC CDR 1 | GGSISSYYWS |
| 21 | 33G12.1-2 HC CDR 2 | RIYTSGSTNYNPSLKS |
| 22 | 33G12.1-2 HC CDR 3 | SGWDNSGSYYSDAFDI |
| 23 | 33G12.1-2 LC | LPVLTQPPSASALLGASIKLTCTLSSEHSTYTIEWYQQRPGRSP |
| | | QYIMKVKSDGSHNKGDGIPDRFMGSSSGADRYLTFSNLQSDDEA |
| | | EYHCGESHTIDGQVGWVFGGGTKLTVL |
| 24 | 33G12.1-2 LC CDR 1 | LSSEHSTYTIE |
| 25 | 33G12.1-2 LC CDR 2 | VKSDGSHNKGD |
| 26 | 33G12.1-2 LC CDR 3 | GESHTIDGQVGWV |
| 27 | 33G12.1-2 scFv | MPLLLLLPLLWAGALAQVQLQESGPGLVKPSETLSLTCTVSGGS |
| | | ISSYYWSWIRQPAGKGLEWIGRIYTSGSTNYNPSLKSRVTMSVD |
| | | TSKNQFSLKLNSVTAADTAVYYCARSGWDNSGSYYSDAFDIWGQ |
| | | GTMVTVSSGGGGSGGGGSGGGGSGGGGSLPVLTQPPSASALLGA |
| | | SIKLTCTLSSEHSTYTIEWYQQRPGRSPQYIMKVKSDGSHNKGD |
| | | GIPDRFMGSSSGADRYLTESNLQSDDEAEYHCGESHTIDGQVGW |
| | | VFGGGTKLTVL |
| 28 | 16C6.6 HC | QVQLQESGPGLVKPSETLSLTCTVSGGSSSSYYWSWIRQSPGKG |
| | | LELIGYIYYSGNTNYNPSLKSRVTISVDTSKKQFSLKLSSVTAA |
| | | DTAVYYCARGGYYDSSGYYLDAFDIWGQGTMVTVSS |
| 29 | 16C6.6 HC CDR 1 | GGSSSSYYWS |
| 30 | 16C6.6 HC CDR 2 | YIYYSGNTNYNPSLKS |
| 31 | 16C6.6 HC CDR 3 | GGYYDSSGYYLDAFDI |
| 32 | 16C6.6 LC | QPVLTQPPSASASLGASVTLTCTLSSGYSNYKVDWYQQRPGKGP |
| | | RFVMRVGTGGIVGSKGDGIPDRFSVLGSGLNRYLTIKNIQEEDE |
| | | SDYHCGADHGSGSNFVYVFGGGTKLTVL |
| 33 | 16C6.6 LC CDR 1 | TLSSGYSNYKVD |
| 34 | 16C6.6 LC CDR 2 | VGTGGIV |
| 35 | 16C6.6 LC CDR 3 | GADHGSGSNFVYV |
| 36 | 16C6.6 scFv | MPLLLLLPLLWAGALAQVQLQESGPGLVKPSETLSLTCTVSGGS |
| | | SSSYYWSWIRQSPGKGLELIGYIYYSGNTNYNPSLKSRVTISVD |
| | | TSKKQFSLKLSSVTAADTAVYYCARGGYYDSSGYYLDAFDIWGQ |
| | | GTMVTVSSGGGGSGGGGSGGGGSGGGGSQPVLTQPPSASASLGA |
| | | SVTLTCTLSSGYSNYKVDWYQQRPGKGPRFVMRVGTGGIVGSKG |
| | | DGIPDRFSVLGSGLNRYLTIKNIQEEDESDYHCGADHGSGSNFV |
| | | YVFGGGTKLTVL |
| 37 | 20H10.1 HC | QVQLLQSGAEVKKPGASVKVSCKASGYTFTNYYMYWVRQAPGQG |
| | | LEWMGIINPGGGGTSYAQMFQGRVTMTRDTSTSTVYMELSSLRS |
| | | EDTAVYYCASPLWVVTAPYYWGQGTLVTVSS |
| 38 | 20H10.1 HC CDR 1 | GYTFTNYYMY |
| 39 | 20H10.1 HC CDR 2 | IINPGGGGTSYAQMFQG |
| 40 | 20H10.1 HC CDR 3 | PLWVVTAPYY |
| 41 | 20H10.1 LC | QPVLTQPPSASASLGASVTLTCTLSSGYSNYKVDWYQQRPGKGP |
| | | RFVMRVGTGGIVGSKGDGIPDRFSVLGSGLNRYLTIKNIQEEDE |
| | | SDYHCGADHGSGSNFVYVFGTGTKVTVL |
| 42 | 20H10.1 LC CDR 1 | TLSSGYSNYKVD |
| 43 | 20H10.1 LC CDR 2 | VGTGGIV |
| 44 | 20H10.1 LC CDR 3 | GADHGSGSNFVYV |
| 45 | 20H10.1 scFv | MPLLLLLPLLWAGALAQVQLLQSGAEVKKPGASVKVSCKASGYT |
| | | FTNYYMYWVRQAPGQGLEWMGIINPGGGGTSYAQMFQGRVTMTR |
| | | DTSTSTVYMELSSLRSEDTAVYYCASPLWVVTAPYYWGQGTLVT |
| | | VSSGGGGSGGGGSGGGGSGGGGSQPVLTQPPSASASLGASVTLT |
| | | CTLSSGYSNYKVDWYQQRPGKGPRFVMRVGTGGIVGSKGDGIPD |
| | | RFSVLGSGLNRYLTIKNIQEEDESDYHCGADHGSGSNFVYVEGT |
| | | GTKVTVL |
| 46 | 13F4.3-1 HC | EVQLVESGGGLVQPGGSLRLSCEASGFTFSSFWMNWVRQAPGKG |
| | | LEWVANIKEDGSEKYYVDSVKGRFTISRDNAKNSLYLQMNSLRV |
| | | EDTAVYYCARALDYYGMDVWGQGTTVTVSS |
| 47 | 13F4.3-1 HC CDR 1 | GFTFSSFWMN |
| 48 | 13F4.3-1 HC CDR 2 | NIKEDGSEKYYVDSVKG |
| 49 | 13F4.3-1 HC CDR 3 | ALDYYGMDV |
| 50 | 13F4.3-1 LC | DIQMTQSPSSLSASVGDRVTITCRASQGISNYLAWYQQKPGKVP |
| | | KLLIYAASTLQSGVPSRFSGSGSGTDETLTISSLQPEDVAIYSC |
| | | QKYNSAPPWTFGQGTKVEIK |
| 51 | 13F4.3-1 LC CDR 1 | RASQGISNYLA |
| 52 | 13F4.3-1 LC CDR 2 | AASTLQS |
| 53 | 13F4.3-1 LC CDR 3 | QKYNSAPPWT |
| 54 | 13F4.3-1 scFv | MPLLLLLPLLWAGALAEVQLVESGGGLVQPGGSLRLSCEASGFT |
| | | FSSFWMNWVRQAPGKGLEWVANIKEDGSEKYYVDSVKGRFTISR |
| | | DNAKNSLYLQMNSLRVEDTAVYYCARALDYYGMDVWGQGTTVTV |
| | | SSGGGGSGGGGSGGGGSGGGGSDIQMTQSPSSLSASVGDRVTIT |
| | | CRASQGISNYLAWYQQKPGKVPKLLIYAASTLQSGVPSRFSGSG |
| | | SGTDFTLTISSLQPEDVAIYSCQKYNSAPPWTFGQGTKVEIK |
| 55 | 13F4.3-2 HC | EVQLVESGGGLVQPGGSLRLSCAASGFTFSSYWMSWVRQAPGKG |
| | | LEWVANIKQDGSERYYVDSVKGRFTISRDNARNSLYLQMNSLRA |
| | | EDMAVYYCAREWYSGSFFDYWGQGTLVTVSS |
| 56 | 13F4.3-2 HC CDR 1 | GFTFSSYWMS |
| 57 | 13F4.3-2 HC CDR 2 | NIKQDGSERYYVDSVKG |
| 58 | 13F4.3-2 HC CDR 3 | EWYSGSFFDY |
| 59 | 13F4.3-2 LC | DIQMTQSPSSLSASVGDRVTITCRASQGISNYLAWYQQKPGKVP |
| | | KLLIYAASTLQSGVPSRFSGSGSGTDFTLTISSLQPEDVAIYSC |
| | | QKYNSAPPWTFGQGTKVEIK |
| 60 | 13F4.3-2 LC CDR 1 | RASQGISNYLA |
| 61 | 13F4.3-2 LC CDR 2 | AASTLQS |
| 62 | 13F4.3-2 LC CDR 3 | QKYNSAPPWT |
| 63 | 13F4.3-2 scFv | MPLLLLLPLLWAGALAEVQLVESGGGLVQPGGSLRLSCAASGFT |
| | | FSSYWMSWVRQAPGKGLEWVANIKQDGSERYYVDSVKGRFTISR |
| | | DNARNSLYLQMNSLRAEDMAVYYCAREWYSGSFFDYWGQGTLVT |
| | | VSSGGGGSGGGGSGGGGSGGGGSDIQMTQSPSSLSASVGDRVTI |
| | | TCRASQGISNYLAWYQQKPGKVPKLLIYAASTLQSGVPSRFSGS |
| | | GSGTDFTLTISSLQPEDVAIYSCQKYNSAPPWTFGQGTKVEIK |
| 64 | 109 HC | EVQLVESGGGLVKPGGSLRLSCAASGFTFSSYSMNWVRQAPGKG |
| | | LEWVSSISGSSNYIYYADSVKGRFTISRDNAKNSLYLQMNSLRA |
| | | EDTAVYYCARGGNYFVEYFQQWGQGTLVTVSS |
| 65 | 109 HC CDR 1 | GFTFSSYSMN |
| 66 | 109 HC CDR 2 | SISGSSNYIYYADSVKG |
| 67 | 109 HC CDR 3 | GGNYFVEYFQQ |
| 68 | i09 LC | EIVLTQSPGTLSLSPGERATLSCRASQYISSNYLAWYQQKPGQA |
| | | PRLLIYGASNRATGIPDRFSGSGSGTDFTLTISRLEPEDFAVYY |
| | | CQQYSSDPITFGQGTKLEIK |
| 69 | 109 LC CDR 1 | RASQYISSNYLA |
| 70 | 109 LC CDR 2 | GASNRAT |
| 71 | 109 LC CDR 3 | QQYSSDPIT |
| 72 | 109 scFv | MPLLLLLPLLWAGALAEVQLVESGGGLVKPGGSLRLSCAASGFT |
| | | FSSYSMNWVRQAPGKGLEWVSSISGSSNYIYYADSVKGRFTISR |
| | | DNAKNSLYLQMNSLRAEDTAVYYCARGGNYFVEYFQQWGQGTLV |
| | | TVSSGGGGSGGGGSGGGGSGGGGSEIVLTQSPGTLSLSPGERAT |
| | | LSCRASQYISSNYLAWYQQKPGQAPRLLIYGASNRATGIPDRFS |
| | | GSGSGTDETLTISRLEPEDFAVYYCQQYSSDPITFGQGTKLEIK |
| 73 | 15B2 HC | EVQLVESGGGLVKPGGSLRLSCAASGFTFRSYSMNWVRQAPGKG |
| | | LEWVSSISGSSNYIYYADSVKGRFTISRDNAKNSLYLQMNSLRA |
| | | EDTAVYYCARGGNYYVEYFQYWGQGTLVTVSS |
| 74 | 15B2 HC CDR 1 | GFTFRSYSMN |
| 75 | 15B2 HC CDR 2 | SISGSSNYIYYADSVKG |
| 76 | 15B2 HC CDR 3 | GGNYYVEYFQY |
| 77 | 15B2 LC | EIVLTQSPGTLSLSPGERATLSCRASQYISSNYLAWYQQKPGQA |
| | | PRLLIYGASNRATGIPDRFSGSGSGTDFTLTISRLEPEDFAVYY |
| | | CQQYGSSPITFGQGTKLEIK |
| 78 | 15B2 LC CDR 1 | RASQYISSNYLA |
| 79 | 15B2 LC CDR 2 | GASNRAT |
| 80 | 15B2 LC CDR 3 | QQYGSSPIT |
| 81 | 15B2 ScFv | MPLLLLLPLLWAGALAEVQLVESGGGLVKPGGSLRLSCAASGGT |
| | | FRSYSMNWVRQAPGKGLEWVSSISGSSNYIYYADSVKGRFTISR |
| | | DNAKNSLYLQMNSLRAEDTAVYYCARGGNYYVEYFQYWGQGTLV |
| | | TVSSGGGGSGGGGSGGGGSGGGGSEIVLTQSPGTLSLSPGERAT |
| | | LSCRASQYISSNYLAWYQQKPGQAPRLLIYGASNRATGIPDRFS |
| | | GSGSGTDETLTISRLEPEDFAVYYCQQYGSSPITFGQGTKLEIK |
| 82 | L15 HC | EVQLVESGGGLVKPGGSLRLSCAASGFTFSSYSMNWVRQAPGKG |
| | | LEWVSSISGQSNYIYYADSVKGRFTISRDNAKNSLYLQMNSLRA |
| | | EDTAVYYCARGGNYFVEYFQYWGQGTLVTVSSGS |
| 83 | L15 HC CDR 1 | GFTFSSYSMN |
| 84 | L15 HC CDR 2 | SISGQSNYIYYADSVKG |
| 85 | L15 HC CDR 3 | GGNYFVEYFQY |
| 86 | L15 LC | EIVLTQSPGTLSLSPGERATLSCRASQYISSNNLAWYQQKPGQA |
| | | PRLLIYGASNRATGIPDRESGSGSGTDETLTISRLEPEDFAVYY |
| | | CQQYADSPITFGQGTKLEIK |
| 87 | L15 LC CDR 1 | RASQYISSNNLA |
| 88 | L15 LC CDR 2 | GASNRAT |
| 89 | L15 LC CDR 3 | QQYADSPIT |
| 90 | L15 ScFv | MPLLLLLPLLWAGALAEVQLVESGGGLVKPGGSLRLSCAASGFT |
| | | FSSYSMNWVRQAPGKGLEWVSSISGQSNYIYYADSVKGRFTISR |
| | | DNAKNSLYLQMNSLRAEDTAVYYCARGGNYFVEYFQYWGQGTLV |
| | | TVSSGSGGGGSGGGGSGGGGSGGGGSEIVLTQSPGTLSLSPGER |
| | | ATLSCRASQYISSNNLAWYQQKPGQAPRLLIYGASNRATGIPDR |
| | | FSGSGSGTDFTLTISRLEPEDFAVYYCQQYADSPITFGQGTKLE |
| | | IK |
| 91 | Signal peptide | MPLLLLLPLLWAGALA |
| 92 | Linker sequence | GGGGSGGGGSGGGGSGGGGS |
| 93 | Hinge | ESKYGPPCPSCP |
| 94 | Transmembrane | FWVLVVVGGVLACYSLLVTVAFIIFWV |
| 95 | Intracellular | KRGRKKLLYIFKQPFMRPVQTTQEEDGCSCRFPEEEEGGCELRV |
| | domain | KESRSADAPAYQQGQNQLYNELNLGRREEYDVLDKRRGRDPEMG |
| | | GKPQRRKNPQEGLYNELQKDKMAEAYSEIGMKGERRRGKGHDGL |
| | | YQGLSTATKDTYDALHMQALPPR |
| 96 | 7A8. 11 Full-length | MPLLLLLPLLWAGALAEVQLVESGGGLVQPGGSLRLSCEASGFT |
| | CAR | FSSFWMNWVRQAPGKGLEWVANIKEDGSEKYYVDSVKGRFTISR |
| | | DNAKNSLYLQMNSLRVEDTAVYYCARALDYYGMDVWGQGTTVTV |
| | | SSGGGGSGGGGSGGGGSGGGGSYIVMTQTPLSLPVTPGEPASIS |
| | | CRSSQSLLDSDDGNTYLDWYLQKPGQSPQLLIYTLSYRASGVPD |
| | | RFSGSGSGTDFTLKISRVEAEDVGVYYCMQRIEFPSYTFGQGTK |
| | | LEIKESKYGPPCPSCPFWVLVVVGGVLACYSLLVTVAFIIFWVK |
| | | RGRKKLLYIFKQPFMRPVQTTQEEDGCSCRFPEEEEGGCELRVK |
| | | FSRSADAPAYQQGQNQLYNELNLGRREEYDVLDKRRGRDPEMGG |
| | | KPQRRKNPQEGLYNELQKDKMAEAYSEIGMKGERRRGKGHDGLY |
| | | QGLSTATKDTYDALHMQALPPR |
| 97 | 16C6. 6 Full-length | MPLLLLLPLLWAGALAQVQLQESGPGLVKPSETLSLTCTVSGGS |
| | CAR | SSSYYWSWIRQSPGKGLELIGYIYYSGNTNYNPSLKSRVTISVD |
| | | TSKKQFSLKLSSVTAADTAVYYCARGGYYDSSGYYLDAFDIWGQ |
| | | GTMVTVSSGGGGSGGGGSGGGGSGGGGSQPVLTQPPSASASLGA |
| | | SVTLTCTLSSGYSNYKVDWYQQRPGKGPRFVMRVGTGGIVGSKG |
| | | DGIPDRFSVLGSGLNRYLTIKNIQEEDESDYHCGADHGSGSNFV |
| | | YVFGGGTKLTVLESKYGPPCPSCPFWVLVVVGGVLACYSLLVTV |
| | | AFIIFWVKRGRKKLLYIFKQPFMRPVQTTQEEDGCSCRFPEEEE |
| | | GGCELRVKESRSADAPAYQQGQNQLYNELNLGRREEYDVLDKRR |
| | | GRDPEMGGKPQRRKNPQEGLYNELQKDKMAEAYSEIGMKGERRR |
| | | GKGHDGLYQGLSTATKDTYDALHMQALPPR |
| 98 | L15 Full-length | MPLLLLLPLLWAGALAEVQLVESGGGLVKPGGSLRLSCAASGFT |
| | CAR | FSSYSMNWVRQAPGKGLEWVSSISGQSNYIYYADSVKGRFTISR |
| | | DNAKNSLYLQMNSLRAEDTAVYYCARGGNYFVEYFQYWGQGTLV |
| | | TVSSGSGGGGSGGGGSGGGGSGGGGSEIVLTQSPGTLSLSPGER |
| | | ATLSCRASQYISSNNLAWYQQKPGQAPRLLIYGASNRATGIPDR |
| | | FSGSGSGTDETLTISRLEPEDFAVYYCQQYADSPITFGQGTKLE |
| | | IKESKYGPPCPSCPFWVLVVVGGVLACYSLLVTVAFIIFWVKRG |
| | | RKKLLYIFKQPFMRPVQTTQEEDGCSCRFPEEEEGGCELRVKES |
| | | RSADAPAYQQGQNQLYNELNLGRREEYDVLDKRRGRDPEMGGKP |
| | | QRRKNPQEGLYNELQKDKMAEAYSEIGMKGERRRGKGHDGLYQG |
| | | LSTATKDTYDALHMQALPPR |

The embodiments described herein can be practiced in the absence of any element or elements, limitation or limitations that are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the embodiments claimed. Thus, it should be understood that although the present description has been specifically disclosed by embodiments, optional features, modification and variation of the concepts herein disclosed may be resorted to by those skilled in the art, and that such modifications and variations are considered to be within the scope of these embodiments as defined by the description and the appended claims. Although some aspects of the present disclosure can be identified herein as particularly advantageous, it is contemplated that the present disclosure is not limited to these particular aspects of the disclosure.

Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein.

All patents and publications mentioned in this specification are herein incorporated by reference to the same extent as if each independent patent and publication was specifically and individually indicated to be incorporated by reference. Citation or identification of any reference in any section of this application shall not be construed as an admission that such reference is available as prior art to the present disclosure.

1. An isolated nucleic acid sequence encoding a chimeric antigen receptor (CAR), wherein the CAR comprises:
(a) an antigen binding domain specific for B cell maturation antigen (BCMA);
(b) a transmembrane domain; and
(c) one or more intracellular domains.

2-18. (canceled)

19. An anti-BCMA chimeric antigen receptor (CAR) comprising an antigen binding domain, wherein the antigen binding domain comprises an antibody, Fab, or an scFv comprising a heavy chain variable region (VH) and a light chain variable region (VL);
wherein the VH comprises a CDR1 comprising an amino acid sequence selected from SEQ ID NO: 2, 29, and 83; a CDR2 comprising an amino acid sequence selected from SEQ ID NO: 3, 30, and 84; and a CDR3 comprising an amino acid sequence selected from SEQ ID NO: 4, 31, and 85; and
wherein the VL comprises a CDR1 comprising an amino acid sequence selected from SEQ ID NO: 6, 33, and 87; a CDR2 comprising an amino acid sequence selected from SEQ ID NO: 7, 34, and 88; and a CDR3 comprising an amino acid sequence selected from SEQ ID NO: 8, 35, and 89.

20. The anti-BCMA CAR of claim 19, wherein the VH comprises an amino acid sequence selected from SEQ ID NO: 1, 28, and 82.

21. The anti-BCMA CAR of claim 19, wherein the VL comprises an amino acid sequence selected from SEQ ID NO: 5, 32, and 86.

22. An anti-BCMA chimeric antigen receptor (CAR) comprising an antigen binding domain, wherein the antigen binding domain comprises an antibody, Fab, or an scFv comprising a heavy chain variable region (VH) and a light chain variable region (VL);
wherein the VH comprises a CDR1 comprising the amino acid sequence of SEQ ID NO: 2; a CDR2 comprising the amino acid sequence of SEQ ID NO: 3; and a CDR3 comprising the amino acid sequence of SEQ ID NO: 4; and
wherein the VL comprises a CDR1 comprising the amino acid sequence of SEQ ID NO: 6; a CDR2 comprising the amino acid sequence of SEQ ID NO: 7; and a CDR3 comprising the amino acid sequence of SEQ ID NO: 8.

23. The anti-BCMA CAR of claim 19, wherein the VH comprises the amino acid sequence of SEQ ID NO: 1, and the VL comprises the amino acid sequence of SEQ ID NO: 5.

24. The anti-BCMA CAR of claim 19, wherein the CAR comprises a transmembrane domain, and one or more intracellular domains.

25. The anti-BCMA CAR of claim 19, wherein the transmembrane domain comprises a transmembrane domain selected from the transmembrane domain of CD4, CD8α, or CD28.

26. The anti-BCMA CAR of claim 25, wherein the transmembrane domain comprises a CD28 transmembrane domain.

27. The anti-BCMA CAR of claim 19, wherein the one or more intracellular domains comprises a costimulatory domain or a portion thereof.

28. The anti-BCMA CAR of claim 27, wherein the costimulatory domain comprises one or more of CD3z, 4-1BB, CD2, CD27, CD28, OX-40, ICOS, IL-2Rβ, GITR, MyD88/CD40a costimulatory domains, and/or variants thereof.

29. The anti-BCMA CAR of claim 24, wherein the intracellular domain comprises a CD3z costimulatory domain and a CD28 costimulatory domain.

30. The anti-BCMA CAR of claim 24, wherein the intracellular domain comprises a CD3z costimulatory domain and a 4-1BB costimulatory domain.

31. The anti-BCMA CAR of claim 24, wherein the intracellular domain comprises a CD3z costimulatory domain, a CD28 costimulatory domain, and a 4-1BB costimulatory domain.

32. The anti-BCMA CAR of claim 19, wherein the CAR further comprises a hinge/spacer domain, optionally, wherein the hinge/spacer domain is located between the antigen binding domain and the transmembrane domain.

33. The anti-BCMA CAR of claim 32, wherein the hinge/spacer domain comprises an IgG1 hinge domain or variants thereof, an IgG2 hinge domain or variants thereof, an IgG3 hinge domain or variants thereof, an IgG4 hinge domain or variants thereof, an IgG4P domain, a CD8a hinge domain or variants thereof, or a CD28 hinge domain or variants thereof.

34. The anti-BCMA CAR of claim 33, wherein the hinge/spacer domain is an IgG4 hinge/spacer, or variants thereof, optionally an IgG4P hinge/spacer comprising an S241P mutation.

35. The anti-BCMA CAR of claim 19, wherein the CAR has an amino acid sequence as set forth in SEQ ID NO: 96.

36. The anti-BCMA CAR of claim 19, wherein the CAR has an amino acid sequence as set forth in SEQ ID NO: 97.

37. The anti-BCMA CAR of claim 19, wherein the CAR has an amino acid sequence as set forth in SEQ ID NO: 98.

38. A vector encoding the chimeric antigen receptor of claim 19, optionally wherein the vector is a virus, a lentivirus, an adenovirus, a retrovirus, an adeno-associated virus (AAV), a transposon, a DNA vector, a mRNA, a lipid nanoparticle (LNP), or a CRISPR-Cas System.

39. A cell comprising the vector of claim 38.

40. A cell comprising a nucleic acid sequence encoding the chimeric antigen receptor (CAR) of claim 19 further comprising decreased expression or knock out of one or more endogenous regulatory factors.

41. The cell of claim 40, wherein the one or more endogenous regulatory factors are selected from cyclin-dependent kinase inhibitor 2A (CDKN2A), cyclin-dependent kinase inhibitor 2B (CDKN2B), and S-methyl-5′-thioadenosine phosphorylase (MTAP).

42. The cell of claim 39, wherein the cell has decreased expression or knock out of CDKN2A, CDKN2B, and MTAP.

43. The cell of claim 39, wherein the cell does not express phosphatase and tensin homolog (PTEN).

44. The cell of claim 39 further comprising a transgene encoding either B-cell lymphoma-extra large (Bcl-xL) or B-cell lymphoma 2 (Bcl-2).

45. The cell of claim 39, wherein the cell does not express of one or more endogenous immune related genes.

46. The cell of claim 45, wherein the endogenous immune related gene is beta-2 microglobulin (B2M), and/or T-cell receptor a constant (TRAC).

47. The cell of claim 39, wherein the cell does not express cluster of differentiation 38 (CD38).

48. A cell comprising a BCMA specific antigen binding domain, wherein the antigen binding domain comprises an antibody, Fab, or an scFv comprising a heavy chain variable region (VH) and a light chain variable region (VL);
wherein the VH comprises a CDR1 comprising an amino acid sequence selected from SEQ ID NO: 2, 29, and 83; a CDR2 comprising an amino acid sequence selected from SEQ ID NO: 3, 30, and 84; and a CDR3 comprising an amino acid sequence selected from SEQ ID NO: 4, 31, and 85; and
wherein the VL comprises a CDR1 comprising an amino acid sequence selected from SEQ ID NO: 6, 33, and 87; a CDR2 comprising an amino acid sequence selected from SEQ ID NO: 7, 34, and 88; and a CDR3 comprising an amino acid sequence selected from SEQ ID NO: 8, 35, and 89.

49-64. (canceled)

65. A method of treating a disease, comprising:
administering to a subject in need thereof an effective amount of a cell comprising an anti-BCMA chimeric antigen receptor (CAR) comprising an antigen binding domain, wherein the antigen binding domain comprises an antibody, Fab, or an scFv comprising a heavy chain variable region (VH) and a light chain variable region (VL),
wherein the VH comprises a CDR1 comprising an amino acid sequence selected from SEQ ID NO: 2, 29, and 83; a CDR2 comprising an amino acid sequence selected from SEQ ID NO: 3, 30, and 84; and a CDR3 comprising an amino acid sequence selected from SEQ ID NO: 4, 31, and 85; and
wherein the VL comprises a CDR1 comprising an amino acid sequence selected from SEQ ID NO: 6, 33, and 87; a CDR2 comprising an amino acid sequence selected from SEQ ID NO: 7, 34, and 88; and a CDR3 comprising an amino acid sequence selected from SEQ ID NO: 8, 35, and 89.

66-85. (canceled)

86. A pharmaceutical composition comprising the anti-BCMA CAR of claim 19 and a pharmaceutically acceptable excipient.

87. A method of treating a disease in a subject in need thereof, comprising administering to the anti-BCMA CAR of claim 19.

88. The method of claim 87, wherein the disease is a cancer or an autoimmune disease.

89. The method of claim 88, wherein the cancer is selected from multiple myeloma (MM), chronic lymphocytic leukemia, acute B-lymphoblastic leukemia, non-Hodgkin lymphoma (NHL), Hodgkin lymphoma, acute myeloid leukemia (AML), and acute lymphoblastic leukemia (ALL).

90. The method of claim 89, wherein the cancer is multiple myeloma.

91. The method of claim 88, wherein the autoimmune disease is lupus.

92-144. (canceled)


SEQUENCE LISTING (98 sequences)

SEQ ID NO: 1 - protein, 118 aa, synthetic construct
EVQLVESGGGLVQPGGSLRLSCEASGFTFSSFWMNWVRQAPGKGLEWVANIKEDGSEKYYVDSVKGRFTISRDNAKNSLYLQMNSLRVEDTAVYYCARALDYYGMDVWGQGTTVTVSS

SEQ ID NO: 2 - protein, 10 aa, synthetic construct
GFTFSSFWMN

SEQ ID NO: 3 - protein, 17 aa, synthetic construct
NIKEDGSEKYYVDSVKG

SEQ ID NO: 4 - protein, 9 aa, synthetic construct
ALDYYGMDV

SEQ ID NO: 5 - protein, 114 aa, synthetic construct
YIVMTQTPLSLPVTPGEPASISCRSSQSLLDSDDGNTYLDWYLQKPGQSPQLLIYTLSYRASGVPDRFSGSGSGTDFTLKISRVEAEDVGVYYCMQRIEFPSYTFGQGTKLEIK

SEQ ID NO: 6 - protein, 17 aa, synthetic construct
RSSQSLLDSDDGNTYLD

SEQ ID NO: 7 - protein, 7 aa, synthetic construct
TLSYRAS

SEQ ID NO: 8 - protein, 10 aa, synthetic construct
MQRIEFPSYT

SEQ ID NO: 9 - protein, 268 aa, synthetic construct
MPLLLLLPLLWAGALAEVQLVESGGGLVQPGGSLRLSCEASGFTFSSFWMNWVRQAPGKGLEWVANIKEDGSEKYYVDSVKGRFTISRDNAKNSLYLQMNSLRVEDTAVYYCARALDYYGMDVWGQGTTVTVSSGGGGSGGGGSGGGGSGGGGSYIVMTQTPLSLPVTPGEPASISCRSSQSLLDSDDGNTYLDWYLQKPGQSPQLLIYTLSYRASGVPDRFSGSGSGTDFTLKISRVEAEDVGVYYCMQRIEFPSYTFGQGTKLEIK

SEQ ID NO: 10 - protein, 124 aa, synthetic construct
QVQLQESGPGLVKPSETLSLTCTVSGGSISSYYWSWIRQPAGKGLEWIGRIYTSGSTNYNPSLKSRVTMSVDTSKNQFSLKLNSVTAADTAVYYCARSGWDNSGSYYSDAFDIWGQGTMVTVSS

SEQ ID NO: 11 - protein, 10 aa, synthetic construct
GGSISSYYWS

SEQ ID NO: 12 - protein, 16 aa, synthetic construct
RIYTSGSTNYNPSLKS

SEQ ID NO: 13 - protein, 16 aa, synthetic construct
SGWDNSGSYYSDAFDI

SEQ ID NO: 14 - protein, 116 aa, synthetic construct
QPVLTQPPSASASLGASVTLTCTLSSGYSNYKVDWYQQRPGKGPRFVMRVGTGGIVGSKGDGIPDRFSVLGSGLNRYLTFKNIQEEAESDYHCGADHGSGSNFVWVFGGGTRLTVL

SEQ ID NO: 15 - protein, 12 aa, synthetic construct
TLSSGYSNYKVD

SEQ ID NO: 16 - protein, 7 aa, synthetic construct
VGTGGIV

SEQ ID NO: 17 - protein, 13 aa, synthetic construct
GADHGSGSNFVWV

SEQ ID NO: 18 - protein, 276 aa, synthetic construct
MPLLLLLPLLWAGALAQVQLQESGPGLVKPSETLSLTCTVSGGSISSYYWSWIRQPAGKGLEWIGRIYTSGSTNYNPSLKSRVTMSVDTSKNQFSLKLNSVTAADTAVYYCARSGWDNSGSYYSDAFDIWGQGTMVTVSSGGGGSGGGGSGGGGSGGGGSQPVLTQPPSASASLGASVTLTCTLSSGYSNYKVDWYQQRPGKGPRFVMRVGTGGIVGSKGDGIPDRFSVLGSGLNRYLTFKNIQEEAESDYHCGADHGSGSNFVWVFGGGTRLTVL

SEQ ID NO: 19 - protein, 124 aa, synthetic construct; identical to SEQ ID NO: 10

SEQ ID NO: 20 - protein, 10 aa, synthetic construct
GGSISSYYWS

SEQ ID NO: 21 - protein, 16 aa, synthetic construct
RIYTSGSTNYNPSLKS

SEQ ID NO: 22 - protein, 16 aa, synthetic construct
SGWDNSGSYYSDAFDI

SEQ ID NO: 23 - protein, 115 aa, synthetic construct
LPVLTQPPSASALLGASIKLTCTLSSEHSTYTIEWYQQRPGRSPQYIMKVKSDGSHNKGDGIPDRFMGSSSGADRYLTFSNLQSDDEAEYHCGESHTIDGQVGWVFGGGTKLTVL

SEQ ID NO: 24 - protein, 11 aa, synthetic construct
LSSEHSTYTIE

SEQ ID NO: 25 - protein, 11 aa, synthetic construct
VKSDGSHNKGD

SEQ ID NO: 26 - protein, 13 aa, synthetic construct
GESHTIDGQVGWV

SEQ ID NO: 27 - protein, 275 aa, synthetic construct
MPLLLLLPLLWAGALAQVQLQESGPGLVKPSETLSLTCTVSGGSISSYYWSWIRQPAGKGLEWIGRIYTSGSTNYNPSLKSRVTMSVDTSKNQFSLKLNSVTAADTAVYYCARSGWDNSGSYYSDAFDIWGQGTMVTVSSGGGGSGGGGSGGGGSGGGGSLPVLTQPPSASALLGASIKLTCTLSSEHSTYTIEWYQQRPGRSPQYIMKVKSDGSHNKGDGIPDRFMGSSSGADRYLTFSNLQSDDEAEYHCGESHTIDGQVGWVFGGGTKLTVL

SEQ ID NO: 28 - protein, 124 aa, synthetic construct
QVQLQESGPGLVKPSETLSLTCTVSGGSSSSYYWSWIRQSPGKGLELIGYIYYSGNTNYNPSLKSRVTISVDTSKKQFSLKLSSVTAADTAVYYCARGGYYDSSGYYLDAFDIWGQGTMVTVSS

SEQ ID NO: 29 - protein, 10 aa, synthetic construct
GGSSSSYYWS

SEQ ID NO: 30 - protein, 16 aa, synthetic construct
YIYYSGNTNYNPSLKS

SEQ ID NO: 31 - protein, 16 aa, synthetic construct
GGYYDSSGYYLDAFDI

SEQ ID NO: 32 - protein, 116 aa, synthetic construct
QPVLTQPPSASASLGASVTLTCTLSSGYSNYKVDWYQQRPGKGPRFVMRVGTGGIVGSKGDGIPDRFSVLGSGLNRYLTIKNIQEEDESDYHCGADHGSGSNFVYVFGGGTKLTVL

SEQ ID NO: 33 - protein, 12 aa, synthetic construct
TLSSGYSNYKVD

SEQ ID NO: 34 - protein, 7 aa, synthetic construct
VGTGGIV

SEQ ID NO: 35 - protein, 13 aa, synthetic construct
GADHGSGSNFVYV

SEQ ID NO: 36 - protein, 276 aa, synthetic construct
MPLLLLLPLLWAGALAQVQLQESGPGLVKPSETLSLTCTVSGGSSSSYYWSWIRQSPGKGLELIGYIYYSGNTNYNPSLKSRVTISVDTSKKQFSLKLSSVTAADTAVYYCARGGYYDSSGYYLDAFDIWGQGTMVTVSSGGGGSGGGGSGGGGSGGGGSQPVLTQPPSASASLGASVTLTCTLSSGYSNYKVDWYQQRPGKGPRFVMRVGTGGIVGSKGDGIPDRFSVLGSGLNRYLTIKNIQEEDESDYHCGADHGSGSNFVYVFGGGTKLTVL

SEQ ID NO: 37 - protein, 119 aa, synthetic construct
QVQLLQSGAEVKKPGASVKVSCKASGYTFTNYYMYWVRQAPGQGLEWMGIINPGGGGTSYAQMFQGRVTMTRDTSTSTVYMELSSLRSEDTAVYYCASPLWVVTAPYYWGQGTLVTVSS

SEQ ID NO: 38 - protein, 10 aa, synthetic construct
GYTFTNYYMY

SEQ ID NO: 39 - protein, 17 aa, synthetic construct
IINPGGGGTSYAQMFQG

SEQ ID NO: 40 - protein, 10 aa, synthetic construct
PLWVVTAPYY

SEQ ID NO: 41 - protein, 116 aa, synthetic construct
QPVLTQPPSASASLGASVTLTCTLSSGYSNYKVDWYQQRPGKGPRFVMRVGTGGIVGSKGDGIPDRFSVLGSGLNRYLTIKNIQEEDESDYHCGADHGSGSNFVYVFGTGTKVTVL

SEQ ID NO: 42 - protein, 12 aa, synthetic construct
TLSSGYSNYKVD

SEQ ID NO: 43 - protein, 7 aa, synthetic construct
VGTGGIV

SEQ ID NO: 44 - protein, 13 aa, synthetic construct
GADHGSGSNFVYV

SEQ ID NO: 45 - protein, 271 aa, synthetic construct
MPLLLLLPLLWAGALAQVQLLQSGAEVKKPGASVKVSCKASGYTFTNYYMYWVRQAPGQGLEWMGIINPGGGGTSYAQMFQGRVTMTRDTSTSTVYMELSSLRSEDTAVYYCASPLWVVTAPYYWGQGTLVTVSSGGGGSGGGGSGGGGSGGGGSQPVLTQPPSASASLGASVTLTCTLSSGYSNYKVDWYQQRPGKGPRFVMRVGTGGIVGSKGDGIPDRFSVLGSGLNRYLTIKNIQEEDESDYHCGADHGSGSNFVYVFGTGTKVTVL

SEQ ID NO: 46 - protein, 118 aa, synthetic construct; identical to SEQ ID NO: 1

SEQ ID NO: 47 - protein, 10 aa, synthetic construct
GFTFSSFWMN

SEQ ID NO: 48 - protein, 17 aa, synthetic construct
NIKEDGSEKYYVDSVKG

SEQ ID NO: 49 - protein, 9 aa, synthetic construct
ALDYYGMDV

SEQ ID NO: 50 - protein, 108 aa, synthetic construct
DIQMTQSPSSLSASVGDRVTITCRASQGISNYLAWYQQKPGKVPKLLIYAASTLQSGVPSRFSGSGSGTDFTLTISSLQPEDVAIYSCQKYNSAPPWTFGQGTKVEIK

SEQ ID NO: 51 - protein, 11 aa, synthetic construct
RASQGISNYLA

SEQ ID NO: 52 - protein, 7 aa, synthetic construct
AASTLQS

SEQ ID NO: 53 - protein, 10 aa, synthetic construct
QKYNSAPPWT

SEQ ID NO: 54 - protein, 262 aa, synthetic construct
MPLLLLLPLLWAGALAEVQLVESGGGLVQPGGSLRLSCEASGFTFSSFWMNWVRQAPGKGLEWVANIKEDGSEKYYVDSVKGRFTISRDNAKNSLYLQMNSLRVEDTAVYYCARALDYYGMDVWGQGTTVTVSSGGGGSGGGGSGGGGSGGGGSDIQMTQSPSSLSASVGDRVTITCRASQGISNYLAWYQQKPGKVPKLLIYAASTLQSGVPSRFSGSGSGTDFTLTISSLQPEDVAIYSCQKYNSAPPWTFGQGTKVEIK

SEQ ID NO: 55 - protein, 119 aa, synthetic construct
EVQLVESGGGLVQPGGSLRLSCAASGFTFSSYWMSWVRQAPGKGLEWVANIKQDGSERYYVDSVKGRFTISRDNARNSLYLQMNSLRAEDMAVYYCAREWYSGSFFDYWGQGTLVTVSS

SEQ ID NO: 56 - protein, 10 aa, synthetic construct
GFTFSSYWMS

SEQ ID NO: 57 - protein, 17 aa, synthetic construct
NIKQDGSERYYVDSVKG

SEQ ID NO: 58 - protein, 10 aa, synthetic construct
EWYSGSFFDY

SEQ ID NO: 59 - protein, 108 aa, synthetic construct; identical to SEQ ID NO: 50

SEQ ID NO: 60 - protein, 11 aa, synthetic construct
RASQGISNYLA

SEQ ID NO: 61 - protein, 7 aa, synthetic construct
AASTLQS

SEQ ID NO: 62 - protein, 10 aa, synthetic construct
QKYNSAPPWT

SEQ ID NO: 63 - protein, 263 aa, synthetic construct
MPLLLLLPLLWAGALAEVQLVESGGGLVQPGGSLRLSCAASGFTFSSYWMSWVRQAPGKGLEWVANIKQDGSERYYVDSVKGRFTISRDNARNSLYLQMNSLRAEDMAVYYCAREWYSGSFFDYWGQGTLVTVSSGGGGSGGGGSGGGGSGGGGSDIQMTQSPSSLSASVGDRVTITCRASQGISNYLAWYQQKPGKVPKLLIYAASTLQSGVPSRFSGSGSGTDFTLTISSLQPEDVAIYSCQKYNSAPPWTFGQGTKVEIK

SEQ ID NO: 64 - protein, 120 aa, synthetic construct
EVQLVESGGGLVKPGGSLRLSCAASGFTFSSYSMNWVRQAPGKGLEWVSSISGSSNYIYYADSVKGRFTISRDNAKNSLYLQMNSLRAEDTAVYYCARGGNYFVEYFQQWGQGTLVTVSS

SEQ ID NO: 65 - protein, 10 aa, synthetic construct
GFTFSSYSMN

SEQ ID NO: 66 - protein, 17 aa, synthetic construct
SISGSSNYIYYADSVKG

SEQ ID NO: 67 - protein, 11 aa, synthetic construct
GGNYFVEYFQQ

SEQ ID NO: 68 - protein, 108 aa, synthetic construct
EIVLTQSPGTLSLSPGERATLSCRASQYISSNYLAWYQQKPGQAPRLLIYGASNRATGIPDRFSGSGSGTDFTLTISRLEPEDFAVYYCQQYSSDPITFGQGTKLEIK

SEQ ID NO: 69 - protein, 12 aa, synthetic construct
RASQYISSNYLA

SEQ ID NO: 70 - protein, 7 aa, synthetic construct
GASNRAT

SEQ ID NO: 71 - protein, 9 aa, synthetic construct
QQYSSDPIT

SEQ ID NO: 72 - protein, 264 aa, synthetic construct
MPLLLLLPLLWAGALAEVQLVESGGGLVKPGGSLRLSCAASGFTFSSYSMNWVRQAPGKGLEWVSSISGSSNYIYYADSVKGRFTISRDNAKNSLYLQMNSLRAEDTAVYYCARGGNYFVEYFQQWGQGTLVTVSSGGGGSGGGGSGGGGSGGGGSEIVLTQSPGTLSLSPGERATLSCRASQYISSNYLAWYQQKPGQAPRLLIYGASNRATGIPDRFSGSGSGTDFTLTISRLEPEDFAVYYCQQYSSDPITFGQGTKLEIK

SEQ ID NO: 73 - protein, 120 aa, synthetic construct
EVQLVESGGGLVKPGGSLRLSCAASGFTFRSYSMNWVRQAPGKGLEWVSSISGSSNYIYYADSVKGRFTISRDNAKNSLYLQMNSLRAEDTAVYYCARGGNYYVEYFQYWGQGTLVTVSS

SEQ ID NO: 74 - protein, 10 aa, synthetic construct
GFTFRSYSMN

SEQ ID NO: 75 - protein, 17 aa, synthetic construct
SISGSSNYIYYADSVKG

SEQ ID NO: 76 - protein, 11 aa, synthetic construct
GGNYYVEYFQY

SEQ ID NO: 77 - protein, 108 aa, synthetic construct
EIVLTQSPGTLSLSPGERATLSCRASQYISSNYLAWYQQKPGQAPRLLIYGASNRATGIPDRFSGSGSGTDFTLTISRLEPEDFAVYYCQQYGSSPITFGQGTKLEIK

SEQ ID NO: 78 - protein, 12 aa, synthetic construct
RASQYISSNYLA

SEQ ID NO: 79 - protein, 7 aa, synthetic construct
GASNRAT

SEQ ID NO: 80 - protein, 9 aa, synthetic construct
QQYGSSPIT

SEQ ID NO: 81 - protein, 264 aa, synthetic construct
MPLLLLLPLLWAGALAEVQLVESGGGLVKPGGSLRLSCAASGFTFRSYSMNWVRQAPGKGLEWVSSISGSSNYIYYADSVKGRFTISRDNAKNSLYLQMNSLRAEDTAVYYCARGGNYYVEYFQYWGQGTLVTVSSGGGGSGGGGSGGGGSGGGGSEIVLTQSPGTLSLSPGERATLSCRASQYISSNYLAWYQQKPGQAPRLLIYGASNRATGIPDRFSGSGSGTDFTLTISRLEPEDFAVYYCQQYGSSPITFGQGTKLEIK

SEQ ID NO: 82 - protein, 122 aa, synthetic construct
EVQLVESGGGLVKPGGSLRLSCAASGFTFSSYSMNWVRQAPGKGLEWVSSISGQSNYIYYADSVKGRFTISRDNAKNSLYLQMNSLRAEDTAVYYCARGGNYFVEYFQYWGQGTLVTVSSGS

SEQ ID NO: 83 - protein, 10 aa, synthetic construct
GFTFSSYSMN

SEQ ID NO: 84 - protein, 17 aa, synthetic construct
SISGQSNYIYYADSVKG

SEQ ID NO: 85 - protein, 11 aa, synthetic construct
GGNYFVEYFQY

SEQ ID NO: 86 - protein, 108 aa, synthetic construct
EIVLTQSPGTLSLSPGERATLSCRASQYISSNNLAWYQQKPGQAPRLLIYGASNRATGIPDRFSGSGSGTDFTLTISRLEPEDFAVYYCQQYADSPITFGQGTKLEIK

SEQ ID NO: 87 - protein, 12 aa, synthetic construct
RASQYISSNNLA

SEQ ID NO: 88 - protein, 7 aa, synthetic construct
GASNRAT

SEQ ID NO: 89 - protein, 9 aa, synthetic construct
QQYADSPIT

SEQ ID NO: 90 - protein, 266 aa, synthetic construct
MPLLLLLPLLWAGALAEVQLVESGGGLVKPGGSLRLSCAASGFTFSSYSMNWVRQAPGKGLEWVSSISGQSNYIYYADSVKGRFTISRDNAKNSLYLQMNSLRAEDTAVYYCARGGNYFVEYFQYWGQGTLVTVSSGSGGGGSGGGGSGGGGSGGGGSEIVLTQSPGTLSLSPGERATLSCRASQYISSNNLAWYQQKPGQAPRLLIYGASNRATGIPDRFSGSGSGTDFTLTISRLEPEDFAVYYCQQYADSPITFGQGTKLEIK

SEQ ID NO: 91 - protein, 16 aa, synthetic construct
MPLLLLLPLLWAGALA

SEQ ID NO: 92 - protein, 20 aa, synthetic construct
GGGGSGGGGSGGGGSGGGGS

SEQ ID NO: 93 - protein, 12 aa, synthetic construct
ESKYGPPCPSCP

SEQ ID NO: 94 - protein, 27 aa, synthetic construct
FWVLVVVGGVLACYSLLVTVAFIIFWV

SEQ ID NO: 95 - protein, 155 aa, synthetic construct
KRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 96 - protein, 462 aa, synthetic construct
MPLLLLLPLLWAGALAEVQLVESGGGLVQPGGSLRLSCEASGFTFSSFWMNWVRQAPGKGLEWVANIKEDGSEKYYVDSVKGRFTISRDNAKNSLYLQMNSLRVEDTAVYYCARALDYYGMDVWGQGTTVTVSSGGGGSGGGGSGGGGSGGGGSYIVMTQTPLSLPVTPGEPASISCRSSQSLLDSDDGNTYLDWYLQKPGQSPQLLIYTLSYRASGVPDRFSGSGSGTDFTLKISRVEAEDVGVYYCMQRIEFPSYTFGQGTKLEIKESKYGPPCPSCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 97 - protein, 470 aa, synthetic construct
MPLLLLLPLLWAGALAQVQLQESGPGLVKPSETLSLTCTVSGGSSSSYYWSWIRQSPGKGLELIGYIYYSGNTNYNPSLKSRVTISVDTSKKQFSLKLSSVTAADTAVYYCARGGYYDSSGYYLDAFDIWGQGTMVTVSSGGGGSGGGGSGGGGSGGGGSQPVLTQPPSASASLGASVTLTCTLSSGYSNYKVDWYQQRPGKGPRFVMRVGTGGIVGSKGDGIPDRFSVLGSGLNRYLTIKNIQEEDESDYHCGADHGSGSNFVYVFGGGTKLTVLESKYGPPCPSCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 98 - protein, 460 aa, synthetic construct
MPLLLLLPLLWAGALAEVQLVESGGGLVKPGGSLRLSCAASGFTFSSYSMNWVRQAPGKGLEWVSSISGQSNYIYYADSVKGRFTISRDNAKNSLYLQMNSLRAEDTAVYYCARGGNYFVEYFQYWGQGTLVTVSSGSGGGGSGGGGSGGGGSGGGGSEIVLTQSPGTLSLSPGERATLSCRASQYISSNNLAWYQQKPGQAPRLLIYGASNRATGIPDRFSGSGSGTDFTLTISRLEPEDFAVYYCQQYADSPITFGQGTKLEIKESKYGPPCPSCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR